(19) United States
(12) Patent Application Publication
(10) Pub. No.: US 2014/0073801 A1
(43) Pub. Date: Mar. 13, 2014

(54) PYRROLIDINE DERIVATIVES AS SELECTIVE GLYCOSIDASE INHIBITORS AND USES THEREOF

(71) Applicant: Summit Corporation Plc, Abingdon (GB)

(72) Inventors: Richard Storer, Abingdon Oxfordshire (GB); Jonathon Mark Tinsley, Abingdon Oxfordshire (GB); Francis Xavier Wilson, Abingdon Oxfordshire (GB); Graeme Horne, Abingdon Oxfordshire (GB); Stephen Paul Wren, Abingdon Oxfordshire (GB); Colin Richard Dorgan, Abingdon Oxfordshire (GB); Renate Maria van Well, Abingdon Oxfordshire (GB); Lindsay Fowler, Abingdon Oxfordshire (GB); Louise Czemerys, Abingdon Oxfordshire (GB)

(21) Appl. No.: 14/013,718

(22) Filed: Aug. 29, 2013

Related U.S. Application Data
(63) Continuation of application No. PCT/GB2012/000200, filed on Mar. 1, 2012.

(30) Foreign Application Priority Data
Mar. 2, 2011 (GB) GB1103526.8

(51) Int. Cl. C07D 207/12; C07D 207/16

(52) U.S. Cl. CPC C07D 207/12; C07D 207/16
USPC 548/537

(57) ABSTRACT

Disclosed are compounds of formula (I):

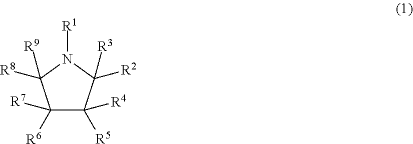

or a pharmaceutically acceptable salt, derivative, solvate, isomer, tautomer, N-oxide, ester, prodrug, isotope or protected form thereof, which are of use as OGA inhibitors, for example in the treatment of various conditions and diseases, including neurodegenerative disorders.

### FIELD OF THE INVENTION

The invention relates to compounds, in particular iminosugars, methods of manufacture of the compounds, and the use of these compounds in the treatment of diseases or disorders responsive to inhibition of O-GlcNAcase. In particular, the invention relates to the use of an iminosugar compound a prodrug thereof and a pharmaceutical composition comprising the compound or the prodrug for selective inhibition of O-GlcNAcase enzymes including human OGA. The invention contemplates the treatment of diseases and disorders related to deficiency or over-expression of O-GlcNAcase, accumulation or deficiency of O-GlcNAc or hyperphosphorylation, including metabolic disorders, neurodegenerative and neurological disorders including tauopathies and synucleinopathies, disorders of the immune system, cardiovascular disorders, infection, cancers, diseases related to cellular stress and inflammatory disorders.

### BACKGROUND OF THE INVENTION

The post-translational modification in which N-acetylglucosamine (GlcNAc) is O-linked to the side chains of serine and threonine residues of cytoplasmic and nuclear proteins is of emerging biological significance. While other forms of protein glycosylation modify proteins on the cell surface or within luminal compartments of the secretory machinery, O-GlcNAc modifies myriad nucleocytoplasmic proteins (over one thousand O-GlcNAc-modified proteins identified to date). These substrates are involved in a multitude of regulatory processes including transcription, proteasomal degradation and cellular signaling, ubiquitination, cell cycle, and stress responses. Unlike the glycosylation of cell-surface or secreted proteins the O-GlcNAc modification is a dynamic process with a relatively high turnover where the addition and removal of O-GlcNAc occurs multiple times during the lifetime of a protein. This imparts dynamic signaling properties to O-GlcNAc comparable to those established for phosphorylation and GlcNAcylation is similar to protein phosphorylation in terms of stoichiometry, localization and cycling.

However, whilst phosphorylation/dephosphorylation is regulated by over 600 enzymes, the reversible nature of the O-GlcNAcylation is modified by the action of only two enzymes: O-linked N-acetylglucosamine transferase (OGT) which catalyses the addition of O-GlcNAc to Ser/Thr residues of target proteins and the neutral hexosaminidase responsible for O-GlcNAc removal O-linked-β-N-acetylglucosaminidase (OGA). OGT installs the O-GlcNAc moiety on target proteins using UDP-GlcNAc (obtained from the hexosamine biosynthetic pathway, HBP) as substrate. OGA returns proteins to the unmodified state through the catalysed removal of O-GlcNAc.

The enzymes of O-GlcNAc cycling couple the nutrient-dependent synthesis of UDP-GlcNAc to the O-GlcNAc modification of Ser/Thr residues. Of all glucose entering into cells 2-5% is directed into HBP towards the synthesis UDP-GlcNAc thereby regulating cellular concentrations. Consequently, levels of UDP-GlcNAc are directly related to glucose uptake and metabolism and any process leading to perturbed glucose utilization will greatly impact on GlcNAc availability and potentially lead to malfunction in O-GlcNAcylated proteins and there associated signalling processes through an increase or decrease in relative GlcNAcylation. Additionally both OGT and OGA are regulated by RNA splicing, nutrients, and post-translational modifications with their specificities controlled by many transiently associated targeting subunits.

As methods for detecting O-GlcNAc have improved our understanding of O-GlcNAc's functions has grown rapidly. All O-GlcNAc modified proteins identified to date can also be modified by phosphorylation. Starting from an un-modified state a protein may become O-GlcNAcylated or phosphorylated. These two modifications regulate each other at the site level by reciprocally influencing site-occupancy, and at the enzymatic-activity level by each modification modulating the catalytic activity of the enzymes involved in the other modification. The specific addition and removal of these two differentially regulated post-translational modifications allows for extensive modification of protein function suggesting a possible interplay between the two modifications which has been observed on many proteins. This GlcNAcylation/phosphorylation interplay may lead to behavioral differences in the modified protein. As glycoproteomics has evolved over the years the number of GlcNAcylated proteins identified has grown considerably as has our understanding of the role of this modification in disease. Abnormal crosstalk between GlcNAcylation and phosphorylation underlies dysregulation in diabetes, including glucose toxicity, and defective GlcNAcylation is involved in cancer, cardiovascular disorders, inflammation, immunoregulation, infection and neurological and neurodegenerative disease.

The O-GlcNAc modification has also been found on many structural and cytoskeletal proteins including neurofilament proteins and synapsins involved in neurodgeneration and neurological disorders including Parkinson's disease (PD), Huntington's disease (HD), synucleinopathies and tauopathies, amyotrophic lateral sclerosis (ALS), multiple sclerosis (MS) and Alzheimer's disease (AD).

It is well established that AD is part characterized by the development of neurofibrillary tangles (NFT) with the number of NFTs correlating with the degree of dementia. NFTs are one of the neuropathological hallmarks of AD and are composed of bundles of paired helical filaments (PHF) the major protein subunit of which is the microtubule-associated protein tau in an abnormally hyperphosphorylated form. Tau is a cytosolic phosphoprotein responsible for stimulating and stabilising the microtubule network. In its hyperphosphorylated form tau is unable to fulfill its normal cellular function and instead aggregates into PHFs. The tau protein contains 85 potential phosphorylation sites in its longest isoform. Over 20 kinases have been found to phosphorylate tau, including proline-directed kinases such as glycogen synthase kinase 3β (GSK3β) and cyclin-dependent kinase 5. Candidate phosphatases include protein phosphatase 1, 2A and 2B (calcineurin). While the phosphorylation of tau appears to be important for function, the importance of individual sites remains unknown. To date >30 phosphorylation sites have been identified in PHF-tau manuy of which have also been identified as sites for O-GlcNAcylation including those that are pathologically relevant to AD. Although only some of these sites are phosphorylated in AD they are to a far greater extent than in normal brain; in cases of confirmed AD PHF-tau phosphorylation levels are 3- to 4-fold higher than those observed in healthy brain tissue. O-GlcNAc levels of soluble tau from AD affected human brains have been shown to be considerably lower than those from healthy subjects. A parallel between the formation of NFTs and the severity of AD clearly exists; however, the precise cause for the hyperphosphorylation of tau and resultant formation of the NFTs is unknown although it may arise from increased activity of the kinases and/or dysfunction of the O-GlcNAc processing enzymes. Although no mutations in the tau gene have yet been implicated in AD several are known to cosegregate with the disease in frontotemporal dementia with Parkinsonism linked to chromosome 17 (FTDP-17) and progressive supranuclear palsy (PSP). The molecular mechanism by which these mutations might lead to disease is not completely understood. It has been shown that four of the FTDP-17 tau mutations, R406W, V337M, G272V, and P301L, result in tau proteins that are more favorable substrates for phosphorylation by brain protein kinases than the wild-type, largest four-repeat protein τ4 L and τ4 L more than τ3 L. At all the sites studied, mutant tau proteins were phosphorylated faster and to a higher extent than τ4L and τ4 L>τ3 L with the most dramatic difference found was in the rate and level of phosphorylation of τ4 L_{R406W} at positions Ser-396, Ser-400, Thr-403, and Ser-404. Phosphorylation of this mutant tau was 12 times faster and 400% greater at Ser-396, a known site of O-GlcNAcylation, and less than 30% at Ser-400, Thr-403, and Ser-404 than phosphorylation of τ 4 L. These findings suggest that the mutations in tau might cause neurodegeneration by making the protein a more favourable substrate for hyperphosphorylation. Additionally within the brains of AD patients a marked decrease in glucose supply and subsequent utilization has been observed and is believed to be a potential cause of neurodegeneration. Furthermore, it has recently been established that the degree of tau phosphorylation is regulated by the relative extent of O-GlcNAcylation. The reciprocal relationship between phosphorylation and O-GlcNAcylation has been detected at both global levels as well as at specific sites with O-GlcNAcylation negatively regulating the majority of phosphorylation sites. As a consequence of this it can be speculated that these two modifications exist in dynamic equilibrium with attenuation of tau phosphorylation levels considered to offer a route to slowing or even halting disease progression in AD and related tauopathies.

Hyperphosphorylated tau has also been identified in chronic experimental autoimmune encephalomyelitis (CEAE) and multiple sclerosis (MS). Multiple sclerosis (MS) is the commonest cause of acquired neurological disability in young adults in the industrialised world. Approximately 10-15% of patients experience a relentlessly progressive course from the outset. Primary progressive multiple sclerosis (PPMS) is characterised by disease progression and accumulation of irreversible disability from presentation in the absence of discrete relapses. In contrast to relapsing-remitting MS, there are no disease-modifying treatments for PPMS. Accumulating evidence from detailed morphological and histological studies suggest that the principal pathological substrate of clinical progression in multiple sclerosis is neuroaxonal degeneration. However, our understanding of the underlying cellular and molecular mechanisms of neurodegeneration is incomplete. Altered axonal transport features in several classic neurodegenerative disorders, including Alzheimer's disease and progressive supranuclear palsy (PSP), characterised by phosphorylating post-translational modifications of the cytoskeletal protein tau. Tau pathology in these degenerative disorders (e.g. PSP and related tauopathies) is not solely restricted to the neuronal pool, with astrocytes also affected despite the relatively low levels of tau expression. Additionally, pathological tau expression and phosphorylation have been shown to relate to altered catabolism within glial cells leading to axonal degeneration and focal neuronal injury. The precise contribution of the disturbed glial environment is unknown, but several lines of evidence particularly from transgenic models of neurodegeneration suggest that abnormal microglia or astrocytes are necessary for progressive neurodegeneration. Insoluble tau formation has recently been described in SPMS and a positive association between insoluble tau accumulation and neurodegeneration has been demonstrated in experimental models of chronic inflammatory demyelination. More recently abnormally phosphorylated soluble tau with a glial predominance has been described in patients. Furthermore, abnormal phosphorylation of tau has been identified in CEAE and in brain samples from patients with secondary progressive MS. The accumulation of insoluble tau is associated with both neuronal and axonal loss and correlates with progression from relapsing remitting to chronic stages of EAE. Significantly, analysis of secondary progressive multiple sclerosis brain tissue also reveals abnormally phosphorylated tau and the formation of insoluble tau. Together, these observations implicate abnormal tau phosphorylation in the neurodegenerative phase of tissue injury in experimental and human demyelinating disease.

Tau has also been implicated in the disease progression of PD and related α-synucleinopathies including dementia with Lewy bodies (DLB). PD is pathologically defined by loss of dopaminergic neurons from the brain stem and other locations and the presence of Lewy bodies (LB) and aberrant neuritis. Both PD and DLB fall under the spectrum of Lewy body disease (LBD) and are charcacterised through the abnormal accumulation of insoluble α-synuclein in dopaminergic neurons. α-Synuclein is ubiquitously expressed in brain tissue and is enriched in presynaptic nerve terminals. Over-expression of α-synuclein is linked to idiopathic PD whiles its A30P and A53T mutants lead to autosomal dominat forms of familial PD. In pathological states α-synuclein misfolds and aggregates in LB and aberrant neuritis. Triplication and duplication of α-synuclein is a known cause of PD and a genome-wide association study has identified a significant link between the MAPT locus and PD suggesting a genetic link between tau and PD with the population attributable risk percent second only to the α-synuclein SNCA gene.

Despite clinical differences pathological and experimental evidence indicates that there is considerable overlap between the tauopathies and synucleinopathies. In AD over 50% of patients with NFT have α-synuclein containing LB and in AD patients with clinically detected extrapyramidal signs, 50% of patients reveal extensive α-synuclein pathology co-localised in the substantia nigra with p-Tau. Additionally in both PD and DLB p-Tau has been observed. Extensive overlap in α-synuclein and p-Tau pathology has been noted in patients with the A53T mutation, in DLB and in familial frontotemporal dementia and progressive aphasia. In the 1-methyl-4-phenyl-1,2,3,6-tetrahydropyridine (MPTP) PD mouse neurotoxin model it has been demonstrated that increases in α-synuclein can initiate and sustain hyperphosphorylation of tau both in vivo and in vitro with co-precipitation of these proteins. Upon oxidative stress by MPTP α-synuclein induced tau phosphorylation through activation of GSK-3β. Increased tau phosphorylation at S396 has been identified in the synapse-enriched fractions from PD brains. Tauopathic changes have been observed in both PD and PD with dementia (PDD) where increased phosphorylation was observed at pathologically relevant sites including S262, S396 and S202; all known sites for O-GlcNAcylation.

All of this suggests that a malfunction in the mechanism regulating tau GlcNAcylation is key to the formation of NFTs and neurological disease/neurodegeneration and that either blocking tau phosphorylation or enhancing tau GlcNAcylation offers a potential therapeutic strategy for the treatment of AD, PD, MS and related disorders. Evidence for the utility of blocking tau phosphorylation for therapeutic intervention is provided from recent results where levels of soluble aggregated hyperphosphorylated tau were markedly reduced in the tau transgenic mouse model JNPL3 when treated with the tyrosine protein kinase inhibitor K252a. An earlier study using the same transgenic model observed correlation between inhibition of GSK-3 with reduced tauopathy and degeneration. These studies highlight that modulation of tau hyperphosphorylation is a viable strategy for treating AD and other tauopathies. Indeed, attenuation of tau phosphorylation is considered to offer a route to slowing or even halting disease progression in AD patients and accordingly intense efforts are currently focused on developing kinase inhibitors for therapeutic benefit. However, because the main tau phosphorylation kinases regulate many other physiological functions in addition to tau phosphorylation, the design of inhibitors that minimize potential toxicity due to off-target functions will be fundamental to their success.

An alternative approach to limiting tau phosphorylation can be envisaged through modulation of the dynamic balance between O-GlcNAcylation and phosphorylation. By directly inhibiting OGA, O-GlcNAc levels should increase while tau phosphorylation levels decrease thus reducing the formation of PHFs and subsequent NFTs. Recent results have highlighted the benefits of such an approach both in vivo and in vitro. The OGA inhibitor thiamet G has been shown to reduce the phosphorylation of tau at pathologically relevant sites including S396 and T231 in both PC-12 cells and S-D rats. Furthermore the chronic effects of OGA inhibition have been observed in the Tau-P301L mouse AD model. Daily treatment with an OGA inhibitor increased O-GlcNAc levels rapidly and stably with significant improvement in both physiology and behaviour observed indicating that OGA inhibition prolongs the survival, rescues or delays motor impairment and mitigates brainstem breathing defects of ageing Tau-P301L mice.

However, the therapeutic effects of GlcNAc modulation are not restricted to neurodegeneration and neurological disorders. Recent evidence has indicated that fluctuations in the levels of O-GlcNAcylated proteins are implicated in cancer, diabetes, inflammation, immunoregulation, infection and cardiovascular disorders. For example, administration of glucosamine/glutamine before ischemia results in enhanced functional recovery and decreased tissue injury following ischemia in the perfused heart with these effects mediated by elevated levels of protein O-GlcNAcylation. More recently inhibition of OGA in perfused rat hearts at the time of reperfusion has been shown to be cadrioprotective in an O-GlcNAc dependent manner.

Several small molecule inhibitors of OGA have been reported including diabetogenic streptozotocin, thiazoline derivatives, and derivatives of the natural product iminosugar NAGstatin as well as acetamido azepanes. Human OGA (hOGA) has two distinct domains: the N terminus contains a glycoside hydrolase while the C-terminus is reported as being a putative histone deacetylase. No structure is currently available for eukaryotic OGA but significant structural and mechanistic insights have been obtained through the solution of several crystal structures of bacterial homologs notably those from Clostridum perfringens and Bacteroides thetaiotaomicron. Bacterial OGA models share significant homology to the hOGA N-terminus domain displaying either identity or high conservation of amino acids within the active site. The catalytic apparatus of these enzymes is similar to the other GH families in which a neighbouring group participation mechanism is used for glycoside hydrolysis including the hexosaminidases Hex-A/B/S and the chitinases, hyaluronidases and endo-β-acetylglucosaminidase. In this process two key catalytic residues are implicated in catalysis. Firstly, the general catalytic acid/base residue transfers a proton to the leaving group facilitating departure. In the second step this residue then acts as a base to facilitate attack of an incoming hydrolytic water molecule. The second residue facilitates the formation and breakdown of the intermediate oxazoline.

This mechanistic process opens up new strategies for the design of selective inhibitors however due to the shared catalytic mechanism with other GH families selectivity is key for this strategy. Despite relatively potent activity against OGA many inhibitors also inhibit members of the GH20 family such as lysosomal hexosaminidases Hex-A/B.

Furthermore, the recent discovery of an additional mammalian cytoplasmic hexosaminidase HexDC increases the need for potent and selective inhibitors of OGA for therapeutic application. However, the therapeutic utility of OGA inhibitors will also in part be affected by inhibition of carbohydrate metabolising enzymes that follow both a neighbouring group catalytic process as well as those that follow a non-neighbouring group participatory mechanism.

The present invention addresses this need and provides, in part, compounds for selectively inhibiting glycosidases, prodrugs of the compounds, uses of the compounds and the prodrugs, pharmaceutical compositions including the compounds or prodrugs, and methods of treating diseases and disorders related to deficiency or overexpression of OGA, and/or accumulation or deficiency of O-GlcNAc.

### SUMMARY OF THE INVENTION

According to a first aspect of the present invention there is provided a compound of Formula (1)

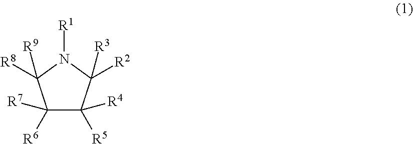

in which
R¹ represents H; C1-15 alkyl, C1-15 alkenyl or C1-15 alkynyl, optionally substituted with one or more R³; oxygen or an oxygen containing group such that the compound is an N-oxide; C(O)OR¹⁰; C(O)NR¹⁰R¹¹; SO₂NR¹⁰; SO₂R¹⁰; OH; OR¹⁰; or formyl
R² represents C(O)NR¹⁰R¹¹; CR¹⁰R¹¹C(O)NR¹⁰R¹¹; C(S)NR¹⁰R¹¹; CR¹⁰R¹¹C(S)NR¹⁰R¹¹; CF₂NR¹⁰R¹¹; CR¹⁰R¹¹CF₂NR¹⁰R¹¹; C(R¹⁰)═CR¹⁰R¹¹; CR¹⁰R¹¹C(R¹⁰)═CR¹⁰R¹¹; SO₂NR¹⁰R¹¹; CR¹⁰R¹¹SO₂NR¹⁰R¹¹; NR¹⁰C(O)NR¹⁰R¹¹; CR¹⁰R¹¹NR¹⁰C(O)NR¹⁰R¹¹; NR¹⁰C(═NR¹⁰)NR¹⁰R¹¹; CR¹⁰R¹¹NR¹⁰C(═NR¹⁰)NR¹⁰R¹¹; CR¹⁰(CF₃)NR¹⁰R¹¹; NR¹⁰C(O)R¹⁰; NR¹⁰C(S)R¹⁰; CR¹⁰R¹¹NR¹⁰C(O)R¹⁰; CR¹⁰R¹¹NR¹⁰C(S)R¹⁰; NR¹⁰SO₂R¹⁰; CR¹⁰R¹¹NR¹⁰SO₂R¹⁰; NR¹⁰C(CF₃)R¹⁰; CR¹⁰R¹¹NR¹⁰C(CF₃)R¹⁰; NR¹⁰CF₂R¹⁰; CR¹⁰R¹¹NR¹⁰CF₂R¹⁰;
each substituent R³, R⁴, R⁵, R⁶, R⁷, R⁸, R⁹ is selected independently from each other from a group consisting of H; OH; OR¹⁰; ═O; NH₂; N₃; SH; SOₓR¹⁰; halo; CN; NO₂; NR¹⁰R¹¹; (NR¹⁰)NR¹⁰R¹¹; NH(NR¹⁰)NR¹⁰R¹¹; CO₂R¹⁰; OC(O)R¹⁰; P(O)(OR¹⁰)₂; C1-15 alkyl or alkenyl optionally substituted with one or more OH, OR¹⁰, ═O, NH₂, N₃, SH, SOₓR¹⁰, halo, CN, NO₂, NR¹⁰R¹¹, (NR¹⁰)NR¹⁰R¹¹, NH(NR¹⁰)NR¹⁰R¹¹, CO₂R¹⁰, OC(O)R¹⁰, P(O)(OR¹⁰)₂, aryl or carbocyclyl groups; carbocyclyl or aryl, either of which is optionally substituted with one or more OH, OR¹⁰, ═O, NH₂, N₃, SH, SO₉R¹⁰, halo, CN, NO₂, NR¹⁰R¹¹, (NR¹⁰)NR¹⁰R¹¹, NH(NR¹⁰)NR¹⁰R¹¹, CO₂R¹⁰, OC(O)R¹⁰, P(O)(OR¹⁰)₂, C1-9 alkyl optionally substituted with one or more OH, OR¹⁰, ═O, NH₂, N₃, halo, CN, NO₂, NR¹⁰R¹¹, CO₂R¹⁰, aryl or carbocyclyl groups; O-glycosyl; C-glycosyl; O-sulfate; O-phosphate or a group which together with the endocyclic carbon forms a spiro ring
R¹⁰ represents H; C1-6 alkyl, optionally substituted with one or more OH, halo; aryl or C1-4 alkyl optionally substituted with aryl; and
R¹¹ represents H; C1-6 alkyl, optionally substituted with one or more OH
R¹⁰ and R¹¹ may optionally form a 3 to 8 membered ring, containing one or more O, SOₓ or NR¹⁹ groups
x represents an integer from 0 to 2
   or a pharmaceutically acceptable salt, derivative, solvate, isomer, tautomer, N-oxide, ester, prodrug, isotope or protected form thereof
   with the provisos that: (a) two OH groups may not be attached to the same endocyclic carbon atom; (b) where there is only one R³, R⁴, R⁵, R⁶, R⁷, R⁸, R⁹ substituent it contains an oxygen atom directly bonded to an endocyclic carbon atom; (c) any two from R³, R⁴, R⁵, R⁶, R⁷, R⁸, R⁹ substituents may together form an optionally heterocyclic ring (for example a carbocycle, cyclic ether or acetal); and (d) the compound is not selected from the following compounds:

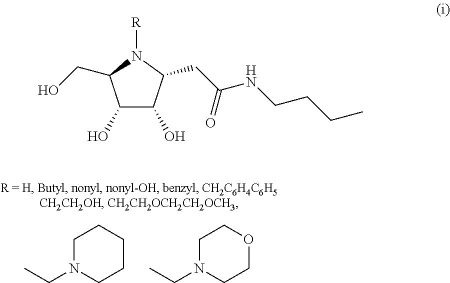

According to a second aspect of the present invention there is provided a compound of Formula (1)

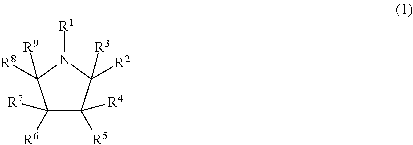

in which
R¹ represents H; C1-15 alkyl, C1-15 alkenyl or C1-15 alkynyl, optionally substituted with one or more R³; oxygen or an oxygen containing group such that the compound is an N-oxide; C(O)OR¹⁰; C(O)NR¹⁰R¹¹; SO₂NR¹⁰; SO₂R¹⁰; OH; OR¹⁰; or formyl
R² represents C(O)NR¹⁰R¹¹; CR¹⁰R¹¹C(O)NR¹⁰R¹¹; C(S)NR¹⁰R¹¹; CR¹⁰R¹¹C(S)NR¹⁰R¹¹; CF₂NR¹⁰R¹¹; CR¹⁰R¹¹CF₂NR¹⁰R¹¹; C(R¹⁰)═CR¹⁰R¹¹; CR¹⁰R¹¹C(R¹⁰)═CR¹⁰R¹¹; SO₂NR¹⁰R¹¹; CR¹⁰R¹¹SO₂NR¹⁰R¹¹; NR¹⁰C(O)NR¹⁰R¹¹; CR¹⁰R¹¹NR¹⁰C(O)NR¹⁰R¹¹; NR¹⁰C(═NR¹⁰)NR¹⁰R¹¹; CR¹⁰R¹¹NR¹⁰C(═NR¹⁰)NR¹⁰R¹¹; CR¹⁰(CF₃)NR¹⁰R¹¹; NR¹⁰C(O)R¹⁰; NR¹⁰C(S)R¹⁰; CR¹⁰R¹¹NR¹⁰C(O)R¹⁰; C R¹⁰R¹¹NR¹⁰C(S)R¹⁰; NR¹⁰SO₂R¹⁰; CR¹⁰R¹¹NR¹⁰SO₂R¹⁰; NR¹⁰C(CF₃)R¹⁰; CR¹⁰R¹¹NR¹⁰C(CF₃)R¹⁰; NR¹⁰CF₂R¹⁰; CR¹⁰R¹¹NR¹⁰CF₂R¹⁰;
each substituent R³, R⁴, R⁵, R⁶, R⁷, R⁸, R⁹ is selected independently from each other from a group consisting of H; OH; OR¹⁰; ═O; NH₂; N₃; SH; SOₓR¹⁰; halo; CN; NO₂; NR¹⁰R¹¹; (NR¹⁰)NR¹⁰R¹¹; NH(NR¹⁰)NR¹⁰R¹¹; CO₂R¹⁰; OC(O)R¹⁰; P(O)(OR¹⁰)₂; C1-15 alkyl or alkenyl optionally substituted with one or more OH, OR¹⁰, ═O, NH₂, N₃, SH, SOₓR¹⁰, halo, CN, NO₂, NR¹⁰R¹¹, (NR¹⁰)NR¹⁰R¹¹, NH(NR¹⁰)NR¹⁰R¹¹, CO₂R¹⁰, OC(O)R¹⁰, P(O)(OR¹⁰)₂, aryl or carbocyclyl groups; carbocyclyl or aryl, either of which is optionally substituted with one or more OH, OR¹⁰, ═O, NH₂, N₃, SH, SOₓR¹⁰, halo, CN, NO₂, NR¹⁰R¹¹, (NR¹⁰)NR¹⁰R¹¹, NH(NR¹⁰)NR¹¹, CO₂R¹⁰, OC(O)R¹⁰, P(O)(OR¹⁰)₂, C1-9 alkyl optionally substituted with one or more OH, OR¹⁰, ═O, NH₂, N₃, halo, CN, NO₂, NR¹⁰R¹¹, CO₂R¹⁰, aryl or carbocyclyl groups; O-glycosyl; C-glycosyl; O-sulfate; O-phosphate or a group which together with the endocyclic carbon forms a spiro ring
R¹⁰ represents H; C1-6 alkyl, optionally substituted with one or more OH, halo; aryl or C1-4 alkyl optionally substituted with aryl; and
R¹¹ represents H; C1-6 alkyl, optionally substituted with one or more OH
R¹⁰ and R¹¹ may optionally form a 3 to 8 membered ring, containing one or more O, SOₓ or NR¹⁰ groups
x represents an integer from 0 to 2
   or a pharmaceutically acceptable salt, derivative, solvate, isomer, tautomer, N-oxide, ester, prodrug, isotope or protected form thereof with the provisos that: (a) two OH groups may not be attached to the same endocyclic carbon atom; (b) where there is only one R³, R⁴, R⁵, R⁶, R⁷, R⁸, R⁹ substituent it contains an oxygen atom directly bonded to an endocyclic carbon atom; and (c) any two from R³, R⁴, R⁵, R⁶, R⁷, R⁸, R⁹ substituents may together form an optionally heterocyclic ring (for example a carbocycle, cyclic ether or acetal);
   for use in any of the methods of therapy or prophylaxis defined herein (see Section headed “Medical uses of the compounds of the invention”).

The compounds defined above and in the claims attached hereto may be comprise a branched ring. Branched compounds are distinguished by the presence of two non-H substituents (e.g. two alkyl groups, two hydroxyalkyl groups, a hydroxy and hydroxyalkyl group or a hydroxy and alkyl group) on any one or more endocyclic carbon atom. For example, in some preferred embodiments the R² substituent is attached to an endocyclic carbon atom which also bears an R³ substituent, for example an hydroxyl group.

Other aspects and preferred embodiments of the invention are defined and described in the claims set out below.

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

### Definitions and General Preferences

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

The phrase “consisting essentially of” is used herein to require the specified integer(s) or steps as well as those which do not materially affect the character or function of the claimed invention.

As used herein, the term “consisting” is used to indicate the presence of the recited integer (e.g. a feature, element, characteristic, property, method/process step or limitation) or group of integers (e.g. features, element, characteristics, properties, method/process steps or limitations) alone.

As used herein the term “β-hexosaminidase” is defined as an enzyme that hydrolyses terminal β-N-acetylglucosamine residues from glycoconjugates.

As used herein the term “glycoside hydrolase” is defined as an enzyme which hydrolyses the glycosidic bond between two or more carbohydrates or between a carbohydrate and a non-carbohydrate moiety.

As used herein the term “inhibits”, or variations thereof such as “inhibition” or “inhibiting” or “inhibitory” are to be defined as a decrease by any value greater than 10%, for example greater than 25%, 50%, 75% or 90%. It is to be understood that inhibiting does not require full inhibition.

As used herein the term “elevating” or “enhancing” or variations thereof such as “elevation” or “elevatory” or “elevated” or “enhancement” or “enhancer” or “enhanced” is defined as an increase by any value greater than 10%, for example greater than 50%, 100%, 200% or 500%, 1000%, 5000% or 10000%.

The term “tauopathy” is a term of art used to define a set of diseases mediated, at least in part, by deficiencies in the microtubule associated protein tau. The term therefore covers aggregative and misfolding tauopathies, including in particular neurodegenerative disorders (e.g. Alzheimer's disease, progressive supranuclear palsy and Frontotemporal dementia and Parkinsonism linked to chromosome 17).

The term “synucleinopathy” is a term of art used to define a set of diseases mediated at least in part by deficiencies in the synuclein protein α-synuclein. The term therefore covers aggregative and misfolding synucleinopathies, including in particular neurodegenerative disorders (e.g. Parkinson's disease, dementia with Lewy bodies and multiple system atrophy).

The term “myelinopathy” is a term of art used to define a set of diseases mediated at least in part by degradation of and/or deficiencies in myelin. The term therefore covers genetic, metabolic and autoimmune myelinopathies (e.g. Multiple Sclerosis, concentric sclerosis and neuromyelitis optica)

As used herein, the term “treatment” or “treating” refers to an intervention (e.g. the administration of an agent to a subject) which cures, ameliorates or lessens the symptoms of a disease or removes (or lessens the impact of) its cause(s) (for example, the reduction in accumulation of pathological levels of lysosomal enzymes). In this case, the term is used synonymously with the term “therapy”.

Additionally, the terms “treatment” or “treating” refers to an intervention (e.g. the administration of an agent to a subject) which prevents or delays the onset or progression of a disease or reduces (or eradicates) its incidence within a treated population. In this case, the term treatment is used synonymously with the term “prophylaxis”. As used herein, the term “disease” is used to define any abnormal condition that impairs physiological function and is associated with specific symptoms. The term is used broadly to encompass any disorder, illness, abnormality, pathology, sickness, condition or syndrome in which physiological function is impaired irrespective of the nature of the aetiology (or indeed whether the aetiological basis for the disease is established). It therefore encompasses conditions arising from infection, trauma, injury, surgery, radiological ablation, poisoning or nutritional deficiencies.

The term “intervention” is a term of art used herein to define any agency which effects a physiological change at any level. Thus, the intervention may comprise the induction or repression of any physiological process, event, biochemical pathway or cellular/biochemical event. The interventions of the invention typically effect (or contribute to) the treatment (i.e. therapy or prophylaxis as herein defined) of a disease and typically involve the administration of an agent to a subject.

In this context “subject” (which is to be read to include “individual”, “animal”, “patient” or “mammal” where context permits) defines any subject, particularly a mammalian subject, for whom treatment is indicated. Mammalian subjects include, but are not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; rodents such as mice, rats, hamsters and guinea pigs; and so on. In preferred embodiments, the subject is a human.

As used herein, an effective amount or a therapeutically effective amount of a compound defines an amount that can be administered to a subject without excessive toxicity, irritation, allergic response, or other problem or complication, commensurate with a reasonable benefit/risk ratio, but one that is sufficient to provide the desired effect, e.g. the treatment or prophylaxis manifested by a permanent or temporary improvement in the subject's condition. The amount will vary from subject to subject, depending on the age and general condition of the individual, mode of administration and other factors. Thus, while it is not possible to specify an exact effective amount, those skilled in the art will be able to determine an appropriate “effective” amount in any individual case using routine experimentation and background general knowledge. A therapeutic result in this context includes eradication or lessening of symptoms, reduced pain or discomfort, prolonged survival, improved mobility and other markers of clinical improvement. A therapeutic result need not be a complete cure.

As used herein, a “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

The term “adjunctive” as applied to the use of the compounds of the invention in therapy or prophylaxis defines uses in which the compound is administered together with one or more other drugs, interventions, regimens or treatments (such as surgery and/or irradiation). Such adjunctive therapies may comprise the concurrent, separate or sequential administration/application of the materials of the invention and the other treatment(s).

Thus, in some embodiments, adjunctive use of the materials of the invention is reflected in the formulation of the pharmaceutical compositions of the invention. For example, adjunctive use may be reflected in a specific unit dosage, or in formulations in which the compound of the invention is present in admixture with the other drug(s) with which it is to be used adjunctively (or else physically associated with the other drug(s) within a single unit dose). In other embodiments, adjunctive use of the compounds or compositions of the invention may be reflected in the composition of the pharmaceutical kits of the invention, wherein the compound of the invention is co-packaged (e.g. as part of an array of unit doses) with the other drug(s) with which it is to be used adjunctively. In yet other embodiments, adjunctive use of the compounds of the invention may be reflected in the content of the information and/or instructions co-packaged with the compound relating to formulation and/or posology.

As used herein, the term “combination”, as applied to two or more compounds and/or agents (also referred to herein as the components), is intended to define material in which the two or more compounds/agents are associated. The terms “combined” and “combining” in this context are to be interpreted accordingly.

The association of the two or more compounds/agents in a combination may be physical or non-physical. Examples of physically associated combined compounds/agents include:
compositions (e.g. unitary formulations) comprising the two or more compounds/agents in admixture (for example within the same unit dose);
compositions comprising material in which the two or more compounds/agents are chemically/physicochemically linked (for example by crosslinking, molecular agglomeration or binding to a common vehicle moiety);
compositions comprising material in which the two or more compounds/agents are chemically/physicochemically co-packaged (for example, disposed on or within lipid vesicles, particles (e.g. micro- or nanoparticles) or emulsion droplets);
pharmaceutical kits, pharmaceutical packs or patient packs in which the two or more compounds/agents are co-packaged or co-presented (e.g. as part of an array of unit doses);

Examples of non-physically associated combined compounds/agents include:
material (e.g. a non-unitary formulation) comprising at least one of the two or more compounds/agents together with instructions for the extemporaneous association of the at least one compound/agent to form a physical association of the two or more compounds/agents;
material (e.g. a non-unitary formulation) comprising at least one of the two or more compounds/agents together with instructions for combination therapy with the two or more compounds/agents;
material comprising at least one of the two or more compounds/agents together with instructions for administration to a patient population in which the other(s) of the two or more compounds/agents have been (or are being) administered;
material comprising at least one of the two or more compounds/agents in an amount or in a form which is specifically adapted for use in combination with the other(s) of the two or more compounds/agents.

As used herein, the term “combination therapy” is intended to define therapies which comprise the use of a combination of two or more compounds/agents (as defined above). Thus, references to “combination therapy”, “combinations” and the use of compounds/agents “in combination” in this application may refer to compounds/agents that are administered as part of the same overall treatment regimen. As such, the posology of each of the two or more compounds/agents may differ: each may be administered at the same time or at different times. It will therefore be appreciated that the compounds/agents of the combination may be administered sequentially (e.g. before or after) or simultaneously, either in the same pharmaceutical formulation (i.e. together), or in different pharmaceutical formulations (i.e. separately). Simultaneously in the same formulation is as a unitary formulation whereas simultaneously in different pharmaceutical formulations is non-unitary. The posologies of each of the two or more compounds/agents in a combination therapy may also differ with respect to the route of administration.

As used herein, the term “pharmaceutical kit” defines an array of one or more unit doses of a pharmaceutical composition together with dosing means (e.g. measuring device) and/or delivery means (e.g. inhaler or syringe), optionally all contained within common outer packaging. In pharmaceutical kits comprising a combination of two or more compounds/agents, the individual compounds/agents may unitary or non-unitary formulations. The unit dose(s) may be contained within a blister pack. The pharmaceutical kit may optionally further comprise instructions for use.

As used herein, the term “pharmaceutical pack” defines an array of one or more unit doses of a pharmaceutical composition, optionally contained within common outer packaging. In pharmaceutical packs comprising a combination of two or more compounds/agents, the individual compounds/agents may unitary or non-unitary formulations. The unit dose(s) may be contained within a blister pack. The pharmaceutical pack may optionally further comprise instructions for use.

As used herein, the term “patient pack” defines a package, prescribed to a patient, which contains pharmaceutical compositions for the whole course of treatment. Patient packs usually contain one or more blister pack(s). Patient packs have an advantage over traditional prescriptions, where a pharmacist divides a patient's supply of a pharmaceutical from a bulk supply, in that the patient always has access to the package insert contained in the patient pack, normally missing in patient prescriptions. The inclusion of a package insert has been shown to improve patient compliance with the physician's instructions.

The combinations of the invention may produce a therapeutically efficacious effect relative to the therapeutic effect of the individual compounds/agents when administered separately.

The term bioisostere (or simply isostere) is a term of art used to define drug analogues in which one or more atoms (or groups of atoms) have been substituted with replacement atoms (or groups of atoms) having similar steric and/or electronic features to those atoms which they replace. The substitution of a hydrogen atom or a hydroxyl group with a fluorine atom is a commonly employed bioisosteric replacement. Sila-substitution (C/Si-exchange) is a relatively recent technique for producing isosteres. This approach involves the replacement of one or more specific carbon atoms in a compound with silicon (for a review, see Tacke and Zilch (1986) Endeavour, New Series 10: 191-197). The sila-substituted isosteres (silicon isosteres) may exhibit improved pharmacological properties, and may for example be better tolerated, have a longer half-life or exhibit increased potency (see for example Englebienne (2005) Med. Chem., 1(3): 215-226). Similarly, replacement of an atom by one of its isotopes, for example hydrogen by deuterium, may also lead to improved pharmacological properties, for example leading to longer half-life (see for example Kushner et al (1999) Can J Physiol Pharmacol. 77(2):79-88). In its broadest aspect, the present invention contemplates all bioisosteres (and specifically, all silicon bioisosteres) of the compounds of the invention.

In its broadest aspect, the present invention contemplates all optical isomers, racemic forms and diastereoisomers of the compounds described herein. Those skilled in the art will appreciate that, owing to the asymmetrically substituted carbon atoms present in the compounds of the invention, the compounds may be produced in optically active and racemic forms. If a chiral centre or another form of isomeric centre is present in a compound of the present invention, all forms of such isomer or isomers, including enantiomers and diastereoisomers, are intended to be covered herein. Compounds of the invention containing a chiral centre (or multiple chiral centres) may be used as a racemic mixture, an enantiomerically enriched mixture, or the racemic mixture may be separated using well-known techniques and an individual enantiomer may be used alone. Thus, references to the compounds (e.g. iminosugars) of the present invention encompass the products as a mixture of diastereoisomers, as individual diastereoisomers, as a mixture of enantiomers as well as in the form of individual enantiomers.

Therefore, the present invention contemplates all optical isomers and racemic forms thereof of the compounds of the invention, and unless indicated otherwise (e.g. by use of dash-wedge structural formulae) the compounds shown herein are intended to encompass all possible optical isomers of the compounds so depicted. In cases where the stereochemical form of the compound is important for pharmaceutical utility, the invention contemplates use of an isolated eutomer.

The terms derivative and pharmaceutically acceptable derivative as applied to the compounds of the invention define compounds which are obtained (or obtainable) by chemical derivatization of the parent compound of the invention. The pharmaceutically acceptable derivatives are therefore suitable for administration to or use in contact with the tissues of humans without undue toxicity, irritation or allergic response (i.e. commensurate with a reasonable benefit/risk ratio). Preferred derivatives are those obtained (or obtainable) by alkylation, esterification or acylation of the parent compounds.

The pharmaceutically acceptable derivatives of the invention may retain some or all of the biological activities described herein. In some cases, the biological activity (e.g. chaperone activity) is increased by derivatization. The derivatives may act as pro-drugs, and one or more of the biological activities described herein (e.g. pharmacoperones activity) may arise only after in vivo processing. Particularly preferred pro-drugs are ester derivatives which are esterified at one or more of the free hydroxyls and which are activated by hydrolysis in vivo. Derivatization may also augment other biological activities of the compound, for example bioavailability and/or glycosidase inhibitory activity and/or glycosidase inhibitory profile. For example, derivatization may increase glycosidase inhibitory potency and/or specificity and/or CNS penetration (e.g. penetration of the blood-brain barrier).

The term pharmaceutically acceptable salt as applied to the iminosugars of the invention defines any non-toxic organic or inorganic acid addition salt of the free base which are suitable for use in contact with the tissues of humans and lower animals without undue toxicity, irritation, allergic response and which are commensurate with a reasonable benefit/risk ratio. Suitable pharmaceutically acceptable salts are well known in the art.

Examples are the salts with inorganic acids (for example hydrochloric, hydrobromic, sulphuric and phosphoric acids), organic carboxylic acids (for example acetic, propionic, glycolic, lactic, pyruvic, malonic, succinic, fumaric, malic, tartaric, citric, ascorbic, maleic, hydroxymaleic, dihydroxymaleic, benzoic, phenylacetic, 4-aminobenzoic, 4-hydroxybenzoic, anthranilic, cinnamic, salicylic, 2-phenoxybenzoic, 2-acetoxybenzoic and mandelic acid) and organic sulfonic acids (for example methanesulfonic acid and p-toluenesulfonic acid).

These salts and the free base compounds can exist in either a hydrated or a substantially anhydrous form. Crystalline forms, including all polymorphic forms, of the iminosugars of the invention are also contemplated and in general the acid addition salts of the compounds are crystalline materials which are soluble in water and various hydrophilic organic solvents and which in comparison to their free base forms, demonstrate higher melting points and an increased solubility.

The term pharmaceutically acceptable metabolite as applied to the compounds of the invention defines a pharmacologically active product produced through metabolism in the body of the specified compound or salt thereof.

The term pharmaceutically acceptable prodrug as applied to the compounds of the invention defines any pharmaceutically acceptable compound that may be converted under physiological conditions or by solvolysis to the specified compound, to a pharmaceutically acceptable salt of such compound or to a compound that shares at least some of the activity of the specified compound.

Prodrugs and active metabolites of the compounds of the invention may be identified using routine techniques known in the art (see for example, Bertolini et al., J. Med. Chem., 1997, 40, 2011-2016).

In the present specification the term “alkyl” defines a straight or branched saturated hydrocarbon chain. The term “C₁-C₆ alkyl” refers to a straight or branched saturated hydrocarbon chain having one to six carbon atoms. The term “C₁-C₉ alkyl” refers to a straight or branched saturated hydrocarbon chain having one to nine carbon atoms. The term “C₁-C₁₅ alkyl” refers to a straight or branched saturated hydrocarbon chain having one to fifteen carbon atoms. Preferred is C₁-C₆ alkyl. Examples include methyl, ethyl, n-propyl, isopropyl, t-butyl, n-hexyl. The alkyl groups of the invention may be optionally substituted by one or more halogen atoms.

In the present specification the term “alkenyl” defines a straight or branched hydrocarbon chain having containing at least one carbon-carbon double bond. The term “C₁-C₆ alkenyl” refers to a straight or branched unsaturated hydrocarbon chain having one to six carbon atoms. The term “C₁-C₉ alkenyl” refers to a straight or branched unsaturated hydrocarbon chain having one to nine carbon atoms. The term “C₁-C₁₅ alkenyl” refers to a straight or branched unsaturated hydrocarbon chain having one to fifteen carbon atoms. Preferred is C₁-C₆ alkenyl. Examples include ethenyl, 2-propenyl, and 3-hexenyl. The alkenyl groups of the invention may be optionally substituted by one or more halogen atoms.

In the present specification the term “alkynyl” defines a straight or branched hydrocarbon chain having containing at least one carbon-carbon triple bond. The term “C₁-C₆ alkynyl” refers to a straight or branched unsaturated hydrocarbon chain having one to six carbon atoms. The term “C₁-C₉ alkynyl” refers to a straight or branched unsaturated hydrocarbon chain having one to nine carbon atoms. The term “C₁-C₁₅ alkynyl” refers to a straight or branched unsaturated hydrocarbon chain having one to fifteen carbon atoms. Preferred is C₁-C₆ alkynyl. Examples include ethynyl, 2-propynyl, and 3-hexynyl. The alkynyl groups of the invention may be optionally substituted by one or more halogen atoms.

As used herein, the term “carbocyclyl” means a mono- or polycyclic residue containing 3 or more (e.g. 3-10 or 3-8) carbon atoms. The carbocyclyl residues of the invention may be optionally substituted by one or more halogen atoms. Mono- and bicyclic carbocyclyl residues are preferred. The carbocyclyl residues can be saturated or partially unsaturated.

Saturated carbocyclyl residues are preferred and are referred to herein as “cycloalkyls” and the term “cycloalkyl” is used herein to define a saturated 3 to 14 membered carbocyclic ring including fused bicyclic or tricyclic systems. Examples of such groups include cyclopropyl, cyclobutyl, cyclopentyl, cyclohexyl and also bridged systems such as norbornyl and adamantyl. The cycloalkyl residues of the invention may be optionally substituted by one or more halogen atoms.

In the present specification the term “aryl” defines a 5-14 (e.g. 5-10) membered aromatic mono-, bi- or tricyclic group at least one ring of which is aromatic. Thus, bicyclic aryl groups may contain only one aromatic ring. As used herein, the term “aryl” includes heteroaryls containing heteroatoms (e.g. nitrogen, sulphur and/or oxygen) being otherwise as defined above. The aryl groups of the invention may optionally be substituted by one or more halogen atoms. Examples of aromatic moieties are benzene, naphthalene, imidazole and pyridine.

In the present specification, “halo” refers to fluoro, chloro, bromo or iodo.

### COMPOUNDS FOR USE ACCORDING TO THE INVENTION

Certain compounds as described herein (e.g. those compounds as defined in the claims attached hereto) are novel.

According to the invention, those compounds which are novel are claimed as compounds per se, together with processes for their preparation, compositions containing them, as well as their use as pharmaceuticals (for example in any of the particular medical uses described herein).

Moreover, to the extent that certain of the compounds as described herein (e.g. those compounds as defined in the claims attached hereto) are known as such but not as pharmaceuticals, those compounds are claimed for use as pharmaceuticals (for example in any of the particular medical uses described herein).

### General Physicochemical Considerations

The compounds for use according to the invention (e.g. those compounds as defined in the claims attached hereto) may have various physicochemical properties.

The compounds for use according to the invention are preferably crystalline materials. Also preferred are compounds which are water soluble, or which are soluble in pharmaceutically acceptable excipients and formulations used in oral or i.v. administration (e.g. those described below). Also preferred are compounds which are subject to efficient passive or active transport to the desired site of action in vivo.

For example, compounds which penetrate the blood brain barrier are preferred for the treatment of e.g. brain tumours.

Preferred are iminosugars having a small molecular weight, since these may exhibit desirable pharmacokinetics. Thus, the iminosugar may have a molecular weight of 100 to 400 Daltons, preferably 150 to 350 Daltons and most preferably 200 to 250 Daltons.

Also preferred are non-metabolizable iminosugars. Such sugars may exhibit extended tissue residence durations, and so exhibit favourable pharmacokinetics.

### Functional Considerations: Target Specificity

The compounds for use according to the invention (e.g. those compounds as defined in the claims attached hereto) may selectively inhibit the target enzyme O-glycoprotein 2-acetamido-2-deoxy-β-D-glucopyranosidase (O-GlcNAcase, OGA) relative to one or more other reference hexosaminidases, for example one or more of HexA, HexB, HexDC and HexS.

The inhibition of target enzyme is preferably greater than 10 fold, and more preferably between 10 and 1×10⁶ fold, or between 100 and 1×10⁶ fold, or between 1000 and 1×10⁶ fold, or between 10,000 and 1×10⁶ fold or between 100,000 and 1×10⁶ fold greater relative to the reference enzyme.

Thus, the compounds for use according to the invention are preferably selective OGA inhibitors. Such selective OGA inhibitors define compounds which exhibit greater inhibition of OGA (preferably human OGA), than of another reference hexosaminidase (for example HexA or HexB, preferably human HexA or human HexB).

Preferred selective OGA inhibitors of the invention exhibit human OGA inhibition which is greater than 10 fold that exhibited against human HexA and/or human HexB.

More preferred are selective OGA inhibitors of the invention which exhibit human OGA inhibition which is between 10 and 1×10⁶ fold, or between 100 and 1×10⁶ fold, or between 1000 and 1×10⁶ fold, or between 10,000 and 1×10⁶ fold or between 100,000 and 1×10⁶ fold greater than that exhibited against human HexA and/or human HexB.

Preferred compounds of the invention:
selectively bind an OGA;
selectively inhibit cleavage of a 2-acetamido-2-deoxy-p-D-glucopyranoside (O-GloNAc);
do not substantially inhibit a mammalian β-hexosaminidase;
do not substantially inhibit a mammalian glycoside hydrolase other than OGA.

Compounds which do not “substantially inhibit” are those which do not exhibit clinically significant levels of enzyme inhibition of the reference enzyme (for example, exhibiting less than 1% inhibition in vitro).

The invention provides, in part, novel compounds that are capable of inhibiting an O-glycoprotein 2-acetamido-2-deoxy-β-D-glucopyranosidase (O-GlcNAcase, OGA). In some embodiments, the OGA is a mammalian OGA, such as a rat, mouse or human OGA. In some embodiments the β-hexosaminidase is a mammalian β-hexosaminidase such as a rat, mouse or human β-hexosaminidase. In some embodiments the glycoside hydrolase is a mammalian glycoside hydrolase such as rat, mouse or human glycoside hydrolase other than OGA.

In some embodiments compounds according to the invention exhibit a surprising and unexpected selectivity in inhibiting an OGA. In some embodiments the compounds according to the invention are surprisingly more selective for an OGA over a β-hexosaminidase or other glycoside hydrolase. In some embodiments, the compounds selectively inhibit the activity of a mammalian OGA over a mammalian β-hexosaminidase or other mammalian glycoside hydrolase. In some embodiments, a selective inhibitor of an OGA does not inhibit substantially a β-hexosaminidase or other glycoside hydrolase.

A compound that “selectively” inhibits an OGA may be a compound that inhibits the activity or biological function of an OGA but does not substantially inhibit the activity or biological function of a β-hexosaminidase or other glycoside hydrolase. For example, in some embodiments, a selective inhibitor of an OGA selectively inhibits the cleavage of a 2-acetamido-2-deoxy-β-D-glucopyranoside (O-GlcNAc) from polypeptides. In some embodiments, a selective inhibitor of an OGA binds to an OGA. In some embodiments a selective inhibitor of an OGA inhibits hyperphosphorylation of a tau protein. In some embodiments, a selective inhibitor of an OGA inhibits formation of NFTs.

In some embodiments a selective inhibitor of an OGA elevates or enhances O-GlcNAc levels, e.g. O-GlcNAc levels on polypeptides or proteins, in cells, tissues, or organs (e.g. in brain, muscle, or cardiac tissue).

In some embodiments, the compounds of the present invention are useful as agents that produce a decrease in tau phosphorylation and NFT formation.

In some embodiments, the compounds produce an increase in levels of O-GlcNAc modification on O-GlcNAc-modified polypeptides or proteins, and are therefore useful for treatment of disorders responsive to such increases in O-GlcNAc modification including without limitation neurodegenerative, neurological, inflammatory, cardiovascular, and immunoregulatory diseases.

In alternative embodiments the compounds of the invention are valuable tools in studying the physiological role of O-GlcNAc at the cellular and organismal level.

### SPECIFIC EXAMPLES

Particular examples of compounds suitable for use according to the invention are listed in Tables 1 (below). References to particular compounds herein refer to the ID codes in these lists.

TABLE 1

| ID | Name |
|---|---|
| A001 | N-butyl-2-((2R,3S,4R,5R)-3,4-dihydroxy-5-(hydroxymethyl)-1-nonylpyrrolidin-2- |
| | yl)acetamide |
| A002 | N-butyl-2-((2R,3S,4R,5R)-3,4-dihydroxy-5-(hydroxymethyl)pyrrolidin-2-yl)acetamide |
| A003 | N-butyl-2-((2R,3S,4R,5R)-3,4-dihydroxy-5-(hydroxymethyl)-1-(9- |
| | hydroxynonyl)pyrrolidin-2-yl)acetamide |
| A004 | 2-((2R,3S,4R,5R)-3,4-dihydroxy-5-(hydroxymethyl)pyrrolidin-2-yl)-N-propylacetamide |
| A005 | 2-((2R,3S,4R,5R)-3,4-dihydroxy-5-(hydroxymethyl)-1-(9-hydroxynonyl)pyrrolidin-2-yl)- |
| | N-propylacetamide |
| A006 | 2-((2R,3S,4R,5R)-3,4-dihydroxy-5-(hydroxymethyl)pyrrolidin-2-yl)-N-ethylacetamide |
| A007 | 2-((2R,3S,4R,5R)-3,4-dihydroxy-5-(hydroxymethyl)pyrrolidin-2-yl)-N-methylacetamide |
| A008 | 2-((2R,3S,4R,5R)-3,4-dihydroxy-5-(hydroxymethyl)-1-nonylpyrrolidin-2-yl)-N- |
| | propylacetamide |
| A009 | 2-((2R,3S,4R,5R)-3,4-dihydroxy-5-(hydroxymethyl)-1-(9-hydroxynonyl)pyrrolidin-2-yl)- |
| | N-methylacetamide |
| A010 | 2-((2R,3S,4R,5R)-3,4-dihydroxy-5-(hydroxymethyl)-1-phenethylpyrrolidin-2-yl)-N- |
| | methylacetamide |
| A011 | 2-((2R,3S,4R,5R)-3,4-dihydroxy-5-(hydroxymethyl)-1-nonylpyrrolidin-2-yl)-N- |
| | methylacetamide |
| A012 | 2-((2R,3S,4R,5R)-1-hexyl-3,4-dihydroxy-5-(hydroxymethyl)pyrrolidin-2-yl)-N- |
| | methylacetamide |
| A013 | 2-((2R,3S,4R,5R)-3,4-dihydroxy-5-(hydroxymethyl)pyrrolidin-2-yl)-N- |
| | isopropylacetamide |
| A014 | 2-((2R,3S,4R,5R)-3,4-dihydroxy-5-(hydroxymethyl)-1-nonylpyrrolidin-2-yl)-N- |
| | isopropylacetamide |
| A015 | 2-((2R,3S,4R,5R)-1-(6-(4-(dimethylamino)naphthalene-1-sulfonamido)hexyl)-3,4- |
| | dihydroxy-5-(hydroxymethyl)pyrrolidin-2-yl)acetamide |
| A016 | 2-((2R,3S,4R,5R)-3,4-dihydroxy-5-(hydroxymethyl)-1-(3-phenylpropyl)pyrrolidin-2-yl)- |
| | N-methylacetamide |
| A017 | 2-((2R,3S,4R,5R)-1-butyl-3,4-dihydroxy-5-(hydroxymethyl)pyrrolidin-2-yl)-N- |
| | methylacetamide |
| A018 | 2-((2R,3S,4R,5R)-3,4-dihydroxy-5-(hydroxymethyl)-1-(4-methylpentyl)pyrrolidin-2-yl)- |
| | N-methylacetamide |
| A019 | 2-((2R,3S,4R,5R)-1-ethyl-3,4-dihydroxy-5-(hydroxymethyl)pyrrolidin-2-yl)-N- |
| | methylacetamide |
| A020 | 2-((2R,3S,4R,5R)-3,4-dihydroxy-5-(hydroxymethyl)-1-(4-phenylbutyl)pyrrolidin-2-yl)-N- |
| | methylacetamide |
| A021 | 2-((2R,3S,4R,5R)-3,4-dihydroxy-5-(hydroxymethyl)-1-(5-methylhexyl)pyrrolidin-2-yl)- |
| | N-methylacetamide |
| A022 | 2-((2R,3S,4R,5R)-3,4-dihydroxy-5-(hydroxymethyl)-1-(4-phenylbutyl)pyrrolidin-2-yl)-N- |
| | propylacetamide |
| A023 | 2-((2R,3S,4R,5R)-1-hexyl-3,4-dihydroxy-5-(hydroxymethyl)pyrrolidin-2-yl)-N- |
| | propylacetamide |
| A024 | 2-((2S,3R,4S,5S)-1-hexyl-3,4-dihydroxy-5-(hydroxymethyl)pyrrolidin-2-yl)-N- |
| | methylacetamide |
| A025 | 2-((2R,3S,4R,5R)-3,4-dihydroxy-5-(hydroxymethyl)pyrrolidin-2-yl)-N- |
| | isobutylacetamide |
| A026 | 2-((2R,3S,4R,5R)-3,4-dihydroxy-5-(hydroxymethyl)-1-(3-phenylpropyl)pyrrolidin-2-yl)- |
| | N-isobutylacetamide |
| A027 | 2-((2R,3S,4R,5R)-1-hexyl-3,4-dihydroxy-5-(hydroxymethyl)pyrrolidin-2-yl)-N- |
| | isobutylacetamide |
| A028 | 2-((2R,3S,4R,5R)-1-hexyl-3,4-dihydroxy-5-(hydroxymethyl)pyrrolidin-2-yl)-N-(2- |
| | hydroxyethyl)acetamide |
| A029 | 2-((2R,3S,4R,5R)-3,4-dihydroxy-5-(hydroxymethyl)-1-(3-phenylpropyl)pyrrolidin-2-yl)- |
| | N-(2-hydroxyethyl)acetamide |
| A030 | 2-((2R,3S,4S,5R)-1-hexyl-3,4-dihydroxy-5-(hydroxymethyl)pyrrolidin-2-yl)-N-(2- |
| | hydroxyethyl)acetamide |
| A031 | N-(cyclohexylmethyl)-2-((2R,3S,4R,5R)-1-hexyl-3,4-dihydroxy-5- |
| | (hydroxymethyl)pyrrolidin-2-yl)acetamide |
| A032 | 2-((2R,3S,4S,5S)-1-hexyl-3,4-dihydroxy-5-(hydroxymethyl)pyrrolidin-2-yl)-N- |
| | methylacetamide |
| A033 | N-(((2R,3S,4R,5R)-3,4-dihydroxy-5-(hydroxymethyl)pyrrolidin-2-yl)methyl)acetamide |
| A034 | 2-((2S,3S,4R,5R)-1-hexyl-3,4-dihydroxy-5-(hydroxymethyl)pyrrolidin-2-yl)-N- |
| | methylacetamide |
| A035 | N-(((2R,3S,4R,5R)-1-hexyl-3,4-dihydroxy-5-(hydroxymethyl)pyrrolidin-2- |
| | yl)methyl)acetamide |
| A036 | 2-((2R,3S,4R,5S)-1-hexyl-3,4-dihydroxy-5-(hydroxymethyl)pyrrolidin-2-yl)-N- |
| | methylacetamide |
| A037 | 2-((2R,3S,4R,5R)-3,4-dihydroxy-5-(hydroxymethyl)-1-(2-phenoxyethyl)pyrrolidin-2-yl)- |
| | N-methylacetamide |
| A038 | N-(((2R,3S,4R,5R)-3,4-dihydroxy-5-(hydroxymethyl)-1-(3-phenylpropyl)pyrrolidin-2- |
| | yl)methyl)acetamide |
| A039 | N-allyl-2-((2R,3S,4R,5R)-1-hexyl-3,4-dihydroxy-5-(hydroxymethyl)pyrrolidin-2- |
| | yl)acetamide |
| A040 | 2-((2R,3S,4R,5R)-3,4-dihydroxy-1-(6-hydroxyhexyl)-5-(hydroxymethyl)pyrrolidin-2-yl)- |
| | N-methylacetamide |
| A041 | 2-((2R,3S,4R,5R)-1-(2-cyclohexylethyl)-3,4-dihydroxy-5-(hydroxymethyl)pyrrolidin-2- |
| | yl)-N-methylacetamide |
| A042 | (2S,3R,4R,5S)-3,4-dihydroxy-5-(hydroxymethyl)-N-methylpyrrolidine-2-carboxamide |
| A043 | (2S,3R,4R)-3,4-dihydroxy-N-methylpyrrolidine-2-carboxamide |
| A044 | 2-((2R,3R,4R,5R)-3,4-dihydroxy-5-(hydroxymethyl)pyrrolidin-2-yl)-N-methylacetamide |
| A045 | 2-((2S,3R,4R,5R)-3,4-dihydroxy-5-(hydroxymethyl)pyrrolidin-2-yl)-N-methylacetamide |
| A046 | 2-((2R,3R,4R,5R)-1-hexyl-3,4-dihydroxy-5-(hydroxymethyl)pyrrolidin-2-yl)-N- |
| | methylacetamide |
| A047 | 2-((2S,3R,4R,5R)-1-hexyl-3,4-dihydroxy-5-(hydroxymethyl)pyrrolidin-2-yl)-N- |
| | methylacetamide |
| A048 | N-cyclopropyl-2-((2R,3S,4R,5R)-3,4-dihydroxy-5-(hydroxymethyl)pyrrolidin-2- |
| | yl)acetamide |
| A049 | N-cyclopropyl-2-((2R,3S,4R,5R)-1-hexyl-3,4-dihydroxy-5-(hydroxymethyl)pyrrolidin-2- |
| | yl)acetamide |
| A050 | 2-((2R,3R,4R,5R)-3,4-dihydroxy-5-(hydroxymethyl)pyrrolidin-2-yl)-N-ethylacetamide |
| A051 | 2-((2S,3R,4R,5R)-3,4-dihydroxy-5-(hydroxymethyl)pyrrolidin-2-yl)-N-ethylacetamide |
| A052 | 2-((2R,3R,4R,5R)-3,4-dihydroxy-5-(hydroxymethyl)pyrrolidin-2-yl)-N-propylacetamide |
| A053 | N-ethyl-2-((2R,3R,4R,5R)-1-hexyl-3,4-dihydroxy-5-(hydroxymethyl)pyrrolidin-2- |
| | yl)acetamide |
| A054 | N-ethyl-2-((2S,3R,4R,5R)-1-hexyl-3,4-dihydroxy-5-(hydroxymethyl)pyrrolidin-2- |
| | yl)acetamide |
| A055 | 2-((2R,3R,4R,5R)-1-hexyl-3,4-dihydroxy-5-(hydroxymethyl)pyrrolidin-2-yl)-N- |
| | propylacetamide |
| A056 | 2-((2R,3R,4R,5S)-1-hexyl-3,4-dihydroxy-5-(hydroxymethyl)pyrrolidin-2-yl)-N- |
| | methylacetamide |
| A057 | 2-((2S,3R,4R,5S)-3,4-dihydroxy-5-(hydroxymethyl)-1-(3-phenylpropyl)pyrrolidin-2-yl)- |
| | N-methylacetamide |
| A058 | 2-((2R,3R,4R,5S)-3,4-dihydroxy-5-(hydroxymethyl)-1-(3-phenylpropyl)pyrrolidin-2-yl)- |
| | N-methylacetamide |
| A059 | (2S,3R,4R,5R)-3,4-dihydroxy-5-(hydroxymethyl)-N-methylpyrrolidine-2-carboxamide |
| A060 | 2-((2R,3R,4R,5R)-3,4-dihydroxy-5-(hydroxymethyl)-1-(3-(4- |
| | methoxyphenyl)propyl)pyrrolidin-2-yl)-N-ethylacetamide |
| A061 | 2-((2R,3R,4R,5R)-3,4-dihydroxy-5-(hydroxymethyl)-1-(3-phenylpropyl)pyrrolidin-2-yl)- |
| | N-ethylacetamide |
| A062 | 2-((2R,3R,4R,5R)-1-(2-(2,4-dichlorophenoxy)ethyl)-3,4-dihydroxy-5- |
| | (hydroxymethyl)pyrrolidin-2-yl)-N-ethylacetamide |
| A063 | 2-((2R,3R,4R,5R)-1-(3-(4-chlorophenyl)propyl)-3,4-dihydroxy-5- |
| | (hydroxymethyl)pyrrolidin-2-yl)-N-ethylacetamide |
| A064 | 2-((2R,3S,4R,5R)-3,4-dihydroxy-5-(hydroxymethyl)-1-(5-(4- |
| | methylphenylsulfonamido)pentyl)pyrrolidin-2-yl)-N-methylacetamide |
| A065 | tert-butyl (3-((2R,3R,4S,5R)-3,4-dihydroxy-2-(hydroxymethyl)-5-(2-(methylamino)-2- |
| | oxoethyl)pyrrolidin-1-yl)propyl)carbamate |
| A066 | 2-((2R,3S,4R,5R)-1-(5-([1,1′-biphenyl]-4-ylsulfonamido)pentyl)-3,4-dihydroxy-5- |
| | (hydroxymethyl)pyrrolidin-2-yl)-N-methylacetamide |
| A067 | 2-((2R,3S,4R,5R)-3,4-dihydroxy-5-(hydroxymethyl)-1-(5-(4- |
| | phenoxyphenylsulfonamido)pentyl)pyrrolidin-2-yl)-N-methylacetamide |
| A068 | 2-((2R,3R,4R,5R)-1-(3,3-diphenylpropyl)-3,4-dihydroxy-5-(hydroxymethyl)pyrrolidin-2- |
| | yl)-N-ethylacetamide |
| A069 | 2-((2R,3R,4R,5R)-3,4-dihydroxy-5-(hydroxymethyl)-1-(3-phenylpropanoyl)pyrrolidin-2- |
| | yl)-N-ethylacetamide |
| A070 | N-ethyl-2-((2R,3R,4R,5R)-1-(3-(4-(N-ethylsulfamoyl)phenyl)propyl)-3,4-dihydroxy-5- |
| | (hydroxymethyl)pyrrolidin-2-yl)acetamide |
| A071 | 2-((2R,3R,4R,5R)-3,4-dihydroxy-5-(hydroxymethyl)-1-(3-(4- |
| | (phenylthio)phenyl)propyl)pyrrolidin-2-yl)-N-ethylacetamide |
| A072 | 2-((2R,3S,4R,5R)-1-(3-([1,1′-biphenyl]-4-ylsulfonamido)propyl)-3,4-dihydroxy-5- |
| | (hydroxymethyl)pyrrolidin-2-yl)-N-methylacetamide |
| A073 | 2-((2R,3R,4R,5R)-1-cinnamyl-3,4-dihydroxy-5-(hydroxymethyl)pyrrolidin-2-yl)-N- |
| | ethylacetamide |
| A074 | 2-((2R,3R,4R,5R)-3,4-dihydroxy-5-(hydroxymethyl)-1-(3-phenylprop-2-yn-1- |
| | yl)pyrrolidin-2-yl)-N-ethylacetamide |
| A075 | 2-((2S,4S,5R)-4-hydroxy-5-(hydroxymethyl)pyrrolidin-2-yl)-N-methylacetamide |
| A076 | N-ethyl-2-((2S,4S,5R)-4-hydroxy-5-(hydroxymethyl)pyrrolidin-2-yl)acetamide |
| A077 | 2-((2S,4S,5R)-1-hexyl-4-hydroxy-5-(hydroxymethyl)pyrrolidin-2-yl)-N-methylacetamide |
| A078 | N-ethyl-2-((2S,4S,5R)-1-hexyl-4-hydroxy-5-(hydroxymethyl)pyrrolidin-2-yl)acetamide |
| A079 | 2-((2S,4S,5R)-4-hydroxy-5-(hydroxymethyl)-1-(3-phenylpropyl)pyrrolidin-2-yl)-N- |
| | methylacetamide |
| A080 | N-ethyl-2-((2S,4S,5R)-4-hydroxy-5-(hydroxymethyl)-1-(3-phenylpropyl)pyrrolidin-2- |
| | yl)acetamide |
| A081 | 2-((2R,3S,4R,5R)-3-fluoro-4-hydroxy-5-(hydroxymethyl)pyrrolidin-2-yl)-N- |
| | methylacetamide |
| A082 | 2-((2R,3R,4R,5R)-3-fluoro-4-hydroxy-5-(hydroxymethyl)pyrrolidin-2-yl)-N- |
| | methylacetamide |
| A083 | N-ethyl-2-((2R,3R,4R,5R)-3-fluoro-4-hydroxy-5-(hydroxymethyl)pyrrolidin-2- |
| | yl)acetamide |
| A084 | 2-((2R,3S,4R,5R)-3-fluoro-1-hexyl-4-hydroxy-5-(hydroxymethyl)pyrrolidin-2-yl)-N- |
| | methylacetamide |
| A085 | 2-((2R,3S,4R,5R)-3-fluoro-4-hydroxy-5-(hydroxymethyl)-1-(3-phenylpropyl)pyrrolidin- |
| | 2-yl)-N-methylacetamide |
| A086 | 2-((2R,3R,4R,5R)-3-fluoro-1-hexyl-4-hydroxy-5-(hydroxymethyl)pyrrolidin-2-yl)-N- |
| | methylacetamide |
| A087 | N-ethyl-2-((2R,3R,4R,5R)-3-fluoro-1-hexyl-4-hydroxy-5-(hydroxymethyl)pyrrolidin-2- |
| | yl)acetamide |
| A088 | 2-((2R,3R,4R,5R)-3-fluoro-4-hydroxy-5-(hydroxymethyl)-1-(3-phenylpropyl)pyrrolidin- |
| | 2-yl)-N-methylacetamide |
| A089 | N-ethyl-2-((2R,3R,4R,5R)-3-fluoro-4-hydroxy-5-(hydroxymethyl)-1-(3- |
| | phenylpropyl)pyrrolidin-2-yl)acetamide |
| A090 | 2-((2R,3S,4R,5R)-4-hydroxy-5-(hydroxymethyl)-3-methoxypyrrolidin-2-yl)-N- |
| | methylacetamide |
| A091 | 2-((2R,3S,4R,5R)-1-hexyl-4-hydroxy-5-(hydroxymethyl)-3-methoxypyrrolidin-2-yl)-N- |
| | methylacetamide |
| A092 | 2-((2R,3S,4R,5R)-4-hydroxy-5-(hydroxymethyl)-3-methoxy-1-(3- |
| | phenylpropyl)pyrrolidin-2-yl)-N-methylacetamide |
| A093 | (R)-2-((2R,3S,4R,5R)-3,4-dihydroxy-5-(hydroxymethyl)pyrrolidin-2-yl)-N- |
| | methylpropanamide |
| A094 | (R)-2-((2R,3S,4R,5R)-1-hexyl-3,4-dihydroxy-5-(hydroxymethyl)pyrrolidin-2-yl)-N- |
| | methylpropanamide |
| A095 | (R)-2-((2R,3S,4R,5R)-3,4-dihydroxy-5-(hydroxymethyl)-1-(3-phenylpropyl)pyrrolidin-2- |
| | yl)-N-methylpropanamide |
| A096 | 2-((2R,3S,4R,5R)-3,4-dihydroxy-5-(hydroxymethyl)pyrrolidin-2-yl)-N,2- |
| | dimethylpropanamide |
| A097 | 2-((2R,3S,4R,5R)-1-hexyl-3,4-dihydroxy-5-(hydroxymethyl)pyrrolidin-2-yl)-N,2- |
| | dimethylpropanamide |
| A098 | 2-((2R,3S,4R,5R)-3,4-dihydroxy-5-(hydroxymethyl)-1-(3-phenylpropyl)pyrrolidin-2-yl)- |
| | N,2-dimethylpropanamide |
| A099 | (2S,3S,4R,5R)-3,4-dihydroxy-5-(hydroxymethyl)-N-methylpyrrolidine-2-carboxamide |
| A100 | (2S,3S,4R,5R)-N-ethyl-3,4-dihydroxy-5-(hydroxymethyl)pyrrolidine-2-carboxamide |
| A101 | (2S,3S,4R,5R)-3,4-dihydroxy-5-(hydroxymethyl)-N-propylpyrrolidine-2-carboxamide |
| A102 | (2S,3R,4R,5R)-3,4-dihydroxy-5-(hydroxymethyl)-N-methylpyrrolidine-2-carboxamide |
| A103 | (2S,3R,4R,5R)-N-ethyl-3,4-dihydroxy-5-(hydroxymethyl)pyrrolidine-2-carboxamide |
| A104 | (2S,3R,4R,5R)-3,4-dihydroxy-5-(hydroxymethyl)-N-propylpyrrolidine-2-carboxamide |
| A105 | (2S,3S,4R,5R)-1-hexyl-3,4-dihydroxy-5-(hydroxymethyl)-N-methylpyrrolidine-2- |
| | carboxamide |
| A106 | (2S,3S,4R,5R)-N-ethyl-1-hexyl-3,4-dihydroxy-5-(hydroxymethyl)pyrrolidine-2- |
| | carboxamide |
| A107 | (2S,3S,4R,5R)-1-hexyl-3,4-dihydroxy-5-(hydroxymethyl)-N-propylpyrrolidine-2- |
| | carboxamide |
| A108 | (2S,3S,4R,5R)-3,4-dihydroxy-5-(hydroxymethyl)-N-methyl-1-(3- |
| | phenylpropyl)pyrrolidine-2-carboxamide |
| A109 | (2S,3S,4R,5R)-N-ethyl-3,4-dihydroxy-5-(hydroxymethyl)-1-(3-phenylpropyl)pyrrolidine- |
| | 2-carboxamide |
| A110 | (2S,3S,4R,5R)-3,4-dihydroxy-5-(hydroxymethyl)-1-(3-phenylpropyl)-N- |
| | propylpyrrolidine-2-carboxamide |
| A111 | (2S,3R,4R,5R)-1-hexyl-3,4-dihydroxy-5-(hydroxymethyl)-N-methylpyrrolidine-2- |
| | carboxamide |
| A112 | (2S,3R,4R,5R)-N-ethyl-1-hexyl-3,4-dihydroxy-5-(hydroxymethyl)pyrrolidine-2- |
| | carboxamide |
| A113 | (2S,3R,4R,5R)-1-hexyl-3,4-dihydroxy-5-(hydroxymethyl)-N-propylpyrrolidine-2- |
| | carboxamide |
| A114 | (2S,3R,4R,5R)-3,4-dihydroxy-5-(hydroxymethyl)-N-methyl-1-(3- |
| | phenylpropyl)pyrrolidine-2-carboxamide |
| A115 | (2S,3R,4R,5R)-N-ethyl-3,4-dihydroxy-5-(hydroxymethyl)-1-(3- |
| | phenylpropyl)pyrrolidine-2-carboxamide |
| A116 | (2S,3R,4R,5R)-3,4-dihydroxy-5-(hydroxymethyl)-1-(3-phenylpropyl)-N- |
| | propylpyrrolidine-2-carboxamide |
| A117 | (2R,4S,5R)-4-hydroxy-5-(hydroxymethyl)-N-methylpyrrolidine-2-carboxamide |
| A118 | (2R,4S,5R)-N-ethyl-4-hydroxy-5-(hydroxymethyl)pyrrolidine-2-carboxamide |
| A119 | (2R,4S,5R)-1-hexyl-4-hydroxy-5-(hydroxymethyl)-N-methylpyrrolidine-2-carboxamide |
| A120 | (2R,4S,5R)-N-ethyl-1-hexyl-4-hydroxy-5-(hydroxymethyl)pyrrolidine-2-carboxamide |
| A121 | (2R,4S,5R)-4-hydroxy-5-(hydroxymethyl)-N-methyl-1-(3-phenylpropyl)pyrrolidine-2- |
| | carboxamide |
| A122 | (2R,4S,5R)-N-ethyl-4-hydroxy-5-(hydroxymethyl)-1-(3-phenylpropyl)pyrrolidine-2- |
| | carboxamide |
| A123 | 2-((2R,3R,4S,5R)-3,4-dihydroxy-5-(hydroxymethyl)pyrrolidin-2-yl)-N-methylacetamide |
| A124 | 2-((2S,3R,4S,5R)-3,4-dihydroxy-5-(hydroxymethyl)pyrrolidin-2-yl)-N-methylacetamide |
| A125 | 2-((2R,3R,4S,5R)-1-hexyl-3,4-dihydroxy-5-(hydroxymethyl)pyrrolidin-2-yl)-N- |
| | methylacetamide |
| A126 | 2-((2S,3R,4S,5R)-1-hexyl-3,4-dihydroxy-5-(hydroxymethyl)pyrrolidin-2-yl)-N- |
| | methylacetamide |
| A127 | 2-((2R,3R,4S,5R)-3,4-dihydroxy-5-(hydroxymethyl)-1-(3-phenylpropyl)pyrrolidin-2-yl)- |
| | N-methylacetamide |
| A128 | 2-((2S,3R,4S,5R)-3,4-dihydroxy-5-(hydroxymethyl)-1-(3-phenylpropyl)pyrrolidin-2-yl)- |
| | N-methylacetamide |

Further examples of compounds suitable for use according to the invention are pharmaceutically acceptable salts, derivatives, solvates, isomers, tautomers, N-oxides, esters, prodrugs, isotopes and protected forms (preferably the salts or tautomers or isomers or N-oxides or solvates, and more preferably, the salts or tautomers or N-oxides or solvates) of the compounds listed in Table 1 (above).

### Medical Uses of the Compounds of the Invention

The compounds of the invention find general application in the treatment or prophylaxis of any condition that is mediated, directly or indirectly, by an OGA enzyme or by O-GlcNAc levels on O-GlcNAc-modified proteins. Thus, the invention finds application in the treatment or prophylaxis of any condition that is benefited by inhibition of an OGA enzyme or by elevation of O-GlcNAc levels on O-GlcNAc-modified proteins.

The compounds of the invention are also useful in the treatment or prophylaxis of diseases or disorders related to deficiency or over-expression of an OGA or accumulation or depletion of O-GlcNAc, or any diseases or disorders responsive to glycosidase inhibition therapy. The compounds of the invention are also useful in the treatment or prophylaxis of diseases or disorders related to the accumulation or deficiency in the enzyme OGT. The compounds of the invention are also useful as a method of protecting or treating dysregulation of O-GlcNAc modification of O-GlcNAc modified proteins, the dysregulation of which modification results in disease or pathology.

The compounds of the invention therefore find wide application in the treatment or prophylaxis of a range of diseases and disorders, including without limitation the following diseases, conditions and disorders: neurodegenerative disease, a tauopathy, a synucleinopathy, a neurological disorder, cancer, stress, infection, inflammatory disorder, Alzheimer's disease, Amyotrophic lateral sclerosis (ALS), Amyotrophic lateral sclerosis with cognitive impairment (ALSci), Argyrophilic grain dementia, Bluit disease, Corticobasal degeneration (CBI), Dementia pugilistica, Diffuse neurofibtillary tangles with calcification, Down's syndrome, Familial British dementia, Familial Danish dementia, Frontotemporal dementia with parkinsonism linked to chromosome 17 (FTDP-17), Gerstmann-Straussler-Scheinker disease, Guadeloupean parkinsonism, Hallevorden-Spatz disease (neurodegeneration with brain iron accumulation type 1), Multiple system atrophy, Myatonic dystrophy, Niemann-Pick disease (type C), Pallido-ponto-nigral degeneration, Parkinsonism-dementia complex of Guam, Pick's disease (PiD), Post-encephalitic parkinsonism (PEP), Mon diseases (including Creutzfeldt-Jakob Disease (CID), Variant Creutzfeldt-Jakob Disease (vCJD), Fatal Familial Insomnia, and Kum), Progressive supercortical gliosis, Progressive supranuclear palsy (PSP), Richardson's syndrome, Subacute sclerosing panencephalitis, Tangle-only dementia, Huntington's disease, Multiple Sclerosis, Primary progressive multiple sclerosis (PPMS), Secondary progressive multiple sclerosis (SPMS), relapsing remitting multiple sclerosis, progressive relapsing multiple sclerosis, atypical multiple sclerosis variants including Devic's disease, Balo concentric sclerosis, Schilder's diffuse sclerosis and Marburg multiple sclerosis, Parkinson's disease, inflammatory or allergic diseases such as asthma, allergic rhinitis, hypersensitivity lung diseases, hypersensitivity pneumonitis, eosinophilic pneumonias, delayed-type hypersensitivity, atherosclerosis, interstitial lung disease (ILD) (e.g., idiopathic pulmonary fibrosis, or ILD associated with rheumatoid arthritis, systemic lupus erythematosus, ankylosing spondylitis, systemic sclerosis, Sjogren's syndrome, polymyositis or dermatomyositis); systemic anaphylaxis or hypersensitivity responses, drug allergies, insect sting allergies; autoimmune diseases, such as rheumatoid arthritis, psoriatic arthritis, multiple sclerosis, systemic lupus erythematosus, myastenia gravis, glomerulonephritis, autoimmune thyroiditis, graft rejection, including allograft rejection or graft-versus-host disease; inflammatory bowel diseases, such as Crohn's disease and ulcerative colitis; spondyloarthropathies; scleroderma; psoriasis (including T-cell mediated psoriasis) and inflammatory dermatoses such as dermatitis, eczema, atopic dermatitis, allergic contact dermatitis, urticaria; vasculitis (e.g., necrotizing, cutaneous, and hypersensitivity vasculitis); eosinphilic myotis, and eosiniphilic fasciitis; graft rejection, in particular but not limited to solid organ transplants, such as heart, lung, liver, kidney, and pancreas transplants (e.g. kidney and lung allografts); epilepsy; pain; stroke, e.g., neuroprotection following a stroke; cardiovascular diseases, diseases associated with inflammation, myelinopathies and diseases associated with immunosuppression.

In some embodiments, the compounds are therefore useful to treat Alzheimer's disease and related tauopathies, Parkinson's disease and related synucleinopathies, Multiple sclerosis and related myelinopathies by lowering tau phosphorylation and reducing NFT formation as a result of increasing levels of O-GlcNAc.

These medical uses are described in further detail below.

### Inflammation

The compounds of the invention may be antiinflammatory and so may find application in any disorder in which inflammation plays a role in the impairment of physiological function and/or symptoms and/or pain. For example, the compounds of the invention may be used to reduce or eliminate chronic inflammation.

Thus, the compounds of the invention find application in the treatment or prophylaxis of non-localized inflammatory disorders, for example those affecting more than one organ. Such disorders include those arising from immune dysfunction (and may therefore have an autoimmune component). Such conditions include systemic lupus erythematosus (SLE), scleroderma and hypersenstivities.

The compounds of the invention also find application in the treatment or prophylaxis of localized inflammatory disorders, including chronic prostatitis, glomerulonephritis, inflammatory bowel diseases, pelvic inflammatory disease, reperfusion injury, rheumatoid arthritis, transplant rejection, vasculitis, asthma, acne, osteoarthritis, oral mucosal, gastrointestinal inflammation, ocular, nasal and aural inflammation and other steroid responsive inflammatory disorders.

In particular, the compounds of the invention find application in the treatment or prophylaxis of cutaneous inflammatory diseases. These include, for example, actinic keratosis, acne (including acne vulgaris, comedonal, acne rosacea, and nodulocystic acne), allergic contact dermatitis, angioedema, bullous pemiphigoid, cutaneous drug reactions, erythema multiforme, lupus erythrametosus, photodermatitis, psoriatic arthritis, scleroderma and urticaria, psoriasis, dermatitis (e.g. atopic dermatitis), scleroderma, steroid-responsive cutaneous inflammatory disorders (for example uremic pruritus) and skin conditions associated with exposure to radiation, chemotherapy and environmental irritants.

The compounds of the invention also find application in the treatment or prophylaxis of inflammatory autoimmune diseases. Such diseases may involve specific tissues or organs (such as the musculoskeletal tissue, as in rheumatoid arthritis and ankylosing spondylitis), the GI tract (as for example in Crohn's disease and ulcerative colitis), the CNS (as for example in Alzheimer's disease, multiple sclerosis, motor neurone disease, Parkinson's disease and chronic fatigue syndrome), pancreatic beta cells (for example insulin-dependent diabetes mellitus), the adrenal gland (for example Addison's disease), the kidney (for example Goodpasture's syndrome, IgA nephropathy and interstitial nephritis), exocrine glands (for example Sjogren's syndrome and autoimmune pancreatitis) and the skin (for example psoriasis and atopic dermatitis).

Other inflammatory disorders treatable according to the present invention include conditions such as osteoarthritis, periodontal disease, diabetic nephropathy, chronic obstructive pulmonary disease, artherosclerosis, graft versus host disease, chronic pelvic inflammatory disease, endometriosis, chronic hepatitis and tuberculosis.

### Inflammatory Respiratory Disease

The compounds of the invention find application in all forms of inflammatory respiratory disease, including for example the prevention or treatment or prophylaxis of local inflammation of the lung, asthma (including atopic and non-atopic asthma), chronic obstructive pulmonary disease (COPD—including chronic bronchitis and emphysema); interstitial lung disease (ILD) (including fibrosing alveolitis, sarcoidiosis and fibrotic lung diseases), hypersensitivity alveolitis (e.g. allergic alveoliotis, idiopathic ILD and autoimmune alveolitis); infectious pulmonary diseases (including bacterial and viral infections of the respiratory tract); occupational inflammatory diseases (for example asbestosis, pulmonary berylliosis, coal worker's pneumoconiosis, silicosis, byssinosis and Pigeon fancier's lung), fibrotic ILD, pneumonia, bronchiectasis, emphysema, tuberculosis, lung collapse, lung fibrosis, fibrosing alveolitis, Wegener's granulomatosis, cystic fibrosis, sarcoidosis, allergic rhinitis, acute respiratory distress syndrome (ARDS) and intrinsic alveolitis.

### Amyloidoses

Certain proteins can assume a non-native, misfolded β-pleated sheet conformation which accumulates as amyloid fibrils (sometimes referred to as amyloid deposits or plaques) in organs and/or tissues, causing disease. These diseases are collectively known as amyloidoses.

Amyloidoses can be classified clinically as primary, secondary, familial, systemic or isolated. Primary amyloidosis appears without any preceding disease, for example arising from immune cell dysfunction such as multiple myeloma and other immunocyte dyscrasias. Secondary amyloidosis is a sequela of an existing disorder (typically a chronic inflammatory disease). Familial amyloidosis (which includes neuropathic, cardiopathic and or nephropathic forms) arises from an inherited mutation and can now be identified by DNA tests. Systemic forms involve amyloid deposition in plural tissues and/or organs (although the brain is almost never directly involved in systemic amyloidosis), while isolated (or localized) amyloidosis involves a single organ, tissue type or system. Thus, recognized clinical forms include ocular amyloidosis and central nervous system amyloidosis.

Amyloidoses can also be classified according to the chemical type of the amyloid protein. The amyloidoses are referred to with a capital A (for amyloid) followed by an abbreviation for the fibril protein. Thus, AA amyloidosis is characterized by extracellular deposition of fibrils that are composed of fragments of serum amyloid A (SAA) protein, a major acute-phase reactant protein, produced predominantly by hepatocytes. Similarly, AL amyloidosis (also called primary amyloidosis or light chain amyloidosis) is characterized by extracellular deposition of fibrils that are composed of an immunoglobulin light chain or light chain fragment, while ATTR amyloidosis is characterized by extracellular deposition of fibrils consisting of the transport protein transthyretin (TTR). Aβ amyloidosis (which appears in Alzheimer's disease) is characterized by extracellular deposition of fibrils that are composed of β-protein precursor.

Symptoms, prognosis and clinical setting differ greatly between amyloid types. Although about 23 different proteins are known to form amyloid in humans, only a few are associated with clinically significant amyloidosis. The various amyloid proteins and the type of amyloidosis and clinical setting in which they are involved are shown in the table below:

| Amyloidosis | | |
|---|---|---|
| type | Amyloid protein type | Principal Clinical Setting(s) |
| Systemic | Immunoglobulin light | Plasma cell disorders |
| | chains | |
| | Transthyretin (TTR) | Familial amyloid polyneuropathies; senile |
| | | cardiac amyloidosis |
| | Serum amyloid A | Amyloid A (AA) amyloidosis; Inflammation- |
| | | associated amyloidosis; familial mediterranean |
| | | fever |
| | β₂-microglobulin | Dialysis-associated amyloidosis |
| | Immunoglobulin heavy | Systemic amyloidosis |
| | chains | |
| Hereditary | Fibrinogen alpha chain | Familial systemic amyloidosis |
| | Apolipoprotein AI | Familial systemic amyloidosis |
| | Apolipoprotein AII | Familial systemic amyloidosis |
| | Lysozyme | Familial systemic amyloidosis |
| Central | β-protein precursor | Alzheimer's disease; Down's syndrome; |
| nervous | | hereditary cerebral hemorrhage with |
| system | | amyloidosis (Dutch) |
| | Prion protein (AScr or PrP- | Creutzfeldt-Jakob disease; Gerstmann- |
| | 27) | Straussler-Scheinker disease; fatal familial |
| | | insomnia |
| | Cystatin C | hereditary cerebral hemorrhage with |
| | | amyloidosis (Icelandic) |
| | ABri precursor protein | Familial dementia (British) |
| | ADan precursor protein | Familial dementia (Danish) |
| Ocular | Gelsolin | Familial amyloidosis (Finnish) |
| | Lactoferrin | Familial corneal amyloidosis |
| | Keratoepithelin | Familial corneal dystrophies |
| Localized | Calcitonin | Medullary thyroid carcinoma |
| | Amylin | Insulinoma; type 2 diabetes |
| | Atrial natriuretic factor | Atrial amyloidosis |
| | Prolactin | Pituitary amyloid |
| | Keratin | Cutaneous amyloidosis |
| | Medin | Senile aortic amyloidosis |

Amyloid A (AA) amyloidosis is the most common form of systemic amyloidosis worldwide. It occurs in the course of a chronic inflammatory disease of either infectious or noninfectious aetiology, hereditary periodic fevers and with certain neoplasms such as Hodgkin disease and renal cell carcinoma.

Thus, the compounds of the invention find application in the treatment or prophylaxis of any of the various amyloidoses described above (and in particular those listed in the above table).

### Synucleinopathies

Synucleinopathies comprise a diverse group of neurodegenerative diseases characterized by the presence of lesions composed of aggregates of conformational and posttranslational modifications of α-synuclein in certain populations of neurons and glia. Abnormal filamentous aggregates of misfolded α-synuclein protein are the major components of Lewy bodies, dystrophic (Lewy) neurites, and the Papp-Lantos filaments in oligodendroglia and neurons in multiple system atrophy linked to degeneration of affected brain regions. In contrast to the extracellular amyloid plaques found in the brains of Alzheimer's patients, Lewy bodies are intracellular.

The synucleinopathies include Lewy body diseases (LBDs), dementia with Lewy bodies, multiple system atrophy (MSA), Hallervorden-Spatz disease, Parkinson's disease (PD), the Lewy body variant of Alzheimer's disease (LBVAD), neurodegeneration with brain iron accumulation type-1 (NBIA-1), pure autonomic failure, neuroaxonal dystrophy, amytrophic lateral sclerosis and Pick disease and various tauopathies.

Thus, the compounds of the invention find application in the treatment or prophylaxis of Lewy body diseases (LBDs), dementia with Lewy bodies, multiple system atrophy (MSA), Hallervorden-Spatz disease, Parkinson's disease (PD), the Lewy body variant of Alzheimer's disease (LBVAD), neurodegeneration with brain iron accumulation type-1 (NBIA-1), pure autonomic failure, neuroaxonal dystrophy, amytrophic lateral sclerosis and Pick disease and various tauopathies.

### Expanded CAG Repeat Diseases

Certain protein aggregation diseases stem from the expansion of CAG repeats in particular genes with the encoded proteins having corresponding polyglutamine tracts which lead to aggregation and accumulation in the nuclei and cytoplasm of neurons. Aggregated amino-terminal fragments of mutant huntingtin are toxic to neuronal cells and are thought to mediate neurodegeneration.

An example is Huntington's disease (HD). Huntington's disease (HD) is characterized by selective neuronal cell death primarily in the cortex and striatum. It is caused by a CAG repeat expansion in the first exon of the huntingtin gene, which encodes a large protein of unknown function. The CAG repeat is highly polymorphic and varies from 6 to 39 repeats in normal individuals and from 35 to 180 repeats in HD cases.

In addition to HD, CAG expansions have been found in at least seven other inherited neurodegenerative disorders, including for example spinal and bulbar muscular atrophy (SBMA), Kennedy's disease, some forms of amyotrophic lateral sclerosis (ALS), dentatorubral pallidoluysian atrophy (DRPLA) and spinocerebellar ataxia (SCA) types 1, 2, 3, 6 and 7.

Thus, the compounds of the invention find application in the treatment or prophylaxis of HD, SBMA, Kennedy's disease, ALS, DRPLA and SCA (e.g. types 1, 2, 3, 6 and 7).

### Tauopathies

The compounds of the invention find application in the treatment or prophylaxis of tauopathy. The tauopathies are a group of diverse dementias and movement disorders which have as a common pathological feature the presence of intracellular accumulations of abnormal filaments of tau protein. Examples include Down's Syndrome (DS), Corticobasal Degeneration (CBD), Frontotemporal Dementia with Parkinsonism linked to Chromosome 17 (FTDP17), Pick Disease (PiD) and Progressive Supranuclear Palsy (PSP).

### Other Aggregation Diseases

The compounds of the invention find application in the treatment or prophylaxis of protein aggregation diseases in general. A growing body of evidence suggests that the familial form of ALS (fALS) is caused by destabilization of the native structure of SOD1 leading to aggregation. Thus, the invention finds application in the treatment or prophylaxis of the familial form of amyotrophic lateral sclerosis (fALS).

### Myelination Diseases

The compounds of the invention find application in the treatment or prophylaxis of myelination diseases and disorders, as described in more detail below.

Myelin is a vital component of the central and peripheral nervous system (CNS and PNS, respectively). A myelin sheath encases the axons of neurons and forms an insulating layer which enhances the speed and integrity of nerve signals, as well as providing trophic support to the associated axon. The myelin sheath is formed by certain glial cells: oligodendrocytes in the CNS and Schwann cells in the PNS. Damage or loss of the myelin sheath produces significant impairment in sensory, motor and other neurological processes as nerve signal transmission is disrupted, perturbed or blocked.

Diseases of myelin (myelination diseases) are extremely diverse but they share the selective loss, absence or impairment of myelin sheaths with relative sparing of the axons (though axonal loss usually follows extensive demyelination giving rise to a cumulative neurological deficit). Two broad subclasses of myelin disease are recognized: (a) demyelinating; and (b) dysmyelinating (see e.g. Neurodystrophies and Neurolipidoses: Convulsive Disorders (Handbook of Clinical Neurology—Revised) by P. J. Vinken and G. W. Bruyn (Editors).

Demyelinating diseases are sporadic or acquired (although genetic factors may play some role) and are characterized by the destruction of biochemically normal myelin, or of the cells that produce it, by processes which include infections, inflammation, autoimmune destruction, toxins, drugs and osmotic fluxes. For example, destruction or functional impairment of myelin-producing Schwann/oligodendrocytes leads to loss of biochemically normal myelin, an example being progressive multifocal leukoencephalopathy (PML) (caused by JC papovavirus infection of oligodendrocytes). The involvement of inflammatory cells directed against specific antigenic myelin components (which may be partitioned between CNS and PNS) means that most inflammatory-mediated demyelinating diseases involve either the CNS or PNS exclusively.

Demyelinatiing diseases include multiple sclerosis (MS), progressive multifocal leukoencephalopathy (PML), optic neuritis, acute demyelinating encephalomyelitis (post viral and post vaccinal), central pontine myelinolysis (CPM), leukodystrophies such as adrenoleukodystrophy, metachromatic leukodystrophy and Krabbe's disease; Cockayne syndrome, Van der Knapp syndrome; Guillain-Barre Syndrome (GBS); chronic inflammatory demyelinating polyneuropathy (CIDP) and multifocal motor neuropathy (MMN).

In contrast, dysmyelinating diseases are hereditary and characterized by the abnormal development of myelin, such as occurs in Pelizaues Merzbacher syndrome or Alexander's disease.

By far the most important of the myelination diseases is multiple sclerosis (MS). Multiple sclerosis is the most common demyelinating disease of the central nervous system, affecting over 1,000,000 people worldwide and about 500,000 people in the United States. It is an immune-mediated inflammatory disease that attacks myelinated axons in the CNS, destroying both the myelin and the axon in variable degrees. The aetiology of the disease has not been fully elucidated, but it appears to involve a combination of genetic susceptibility and an environmental trigger which resulting in a self-sustaining autoimmune/inflammatory disorder that leads to recurrent immune attacks on the CNS.

The disease is usually characterized by relapses and remissions with accumulating neurological deficit leading eventually to chronic disability. The initial phases of the disease appear to involve an autoimmune inflammatory attack on the myelin sheath, producing symptoms ranging from skin tingling to paralysis, lack of coordination, sensory disturbances and visual impairment. The subsequent chronic progressive phase of the disease is associated with the loss of axons.

Several distinct subclasses of MS are recognized and are used as a basis for both prognosis and therapeutic decisions. Relapsing-remitting MS defines the initial course in 85% to 90% of patients. This subclass is characterized by unpredictable attacks (relapses) followed by periods of months to years of relative freedom from symptoms (remission). Deficits arising during relapse may either resolve or remain. When deficits always completely resolve between attacks with no or only minor accumulation of disability after decades, the disease may be classed as benign MS.

Secondary progressive MS defines a condition to which about 80% of patients with initial relapsing-remitting MS progress. It is characterized by neurological decline between relapses without any periods of remission and appears to be associated with progressive axonal loss. Secondary progressive MS is the most common subclass of MS and causes the greatest amount of disability. Its impact is exacerbated by the fact that it does not seem to be responsive to the available treatments for relapsing-remitting MS (see further discussion below).

Of all multiple sclerosis patients, 10-15% never have a relapsing phase. In these patients decline is continuous with no clear attacks or periods of remission. This subclass is called primary progressive MS. Little or no inflammation is detectable in these patients on neuroimaging and indeed it is debated whether this is a distinct condition. A fourth subclass of MS, progressive relapsing MS, defines a disease course in which a continuous neurological decline is superimposed with relapses. It is the least common of all MS subclasses.

Rare cases of the disease with non-standard behaviour have also been described although they are generally regarded as different diseases. These diseases may be referred to as borderline forms of multiple sclerosis and include Balo concentric sclerosis, Schilder's diffuse sclerosis and Marburg multiple sclerosis.

There is also a distinct condition known as neuromyelitis optica, associated with antibodies to the aquaporin 4 antigen.

The compounds of the invention find application in the treatment or prophylaxis of diseases in which myelination and/or oligodendrocyte progenitor cell proliferation, migration, differentiation and/or promyelinative activity is indicated. This may be the case, for example, in patients exhibiting T2 hyperintensity of white matter tracts on brain MRI. It may also be the case in patients having a peripheral conduction velocity of the median nerve of less than 35 m/s.

Preferred is the treatment or prophylaxis of multiple sclerosis, including: (a) secondary progressive multiple sclerosis; (b) primary progressive multiple sclerosis; and (c) progressive relapsing multiple sclerosis. Particularly preferred is the treatment or prophylaxis of secondary progressive multiple sclerosis.

### Remyelination

The compounds of the invention find application in promoting remyelination. Remyelination is the regenerative process by which demyelinated axons are reinvested with new myelin sheaths. Both the PNS and CNS have an inherent remyelinative potential which is associated with functional recovery (e.g. in the remission phase of MS) and in the maintenance of axonal function. It occurs spontaneously following demyelination in a range of pathologies (including trauma) as well as myelination diseases (including multiple sclerosis, where myelinating oligodendrocytes have been identified at demyelinated lesions indicating axonal repair with newly synthesized myelin).

Thus, the invention finds broad application in the treatment or prophylaxis of diseases in which the stimulation of myelination is indicated. This may be the case, for example, in patients exhibiting T2 hyperintensity of white matter tracts on brain MRI. It may also be the case in patients having a peripheral conduction velocity of the median nerve of less than 35 m/s.

Preferred is the treatment or prophylaxis of multiple sclerosis, including: (a) secondary progressive multiple sclerosis; (b) primary progressive multiple sclerosis; and (c) progressive relapsing multiple sclerosis. Particularly preferred is the treatment or prophylaxis of secondary progressive multiple sclerosis.

However, myelin-related diseases are associated with a partial or total failure of endogenous regenerative remyelinating processes and in multiple sclerosis the progressive forms of the disease (secondary progressive, primary progressive and progressive relapsing MS) have been linked with depletion of the myelin-producing oligodendrocytes (or of their precursors, the oligodendrocyte progenitor cells or OPCS) during the demyelination process, a process which eventually culminates in axonal loss. Thus, while remyelination occurs in many MS lesions it becomes increasingly incomplete/inadequate and eventually fails.

### Demyelinating Diseases

The invention finds broad application in the treatment or prophylaxis of all demyelinative (sometimes referred to as demyelinative) diseases, either of the CNS or PNS alone or (less commonly) demyelinating diseases in which both the CNS and PNS are affected.

Suitable subjects for treatment or prophylaxis may be identified by known diagnostic criteria. Morphologically, neuronal demyelination can be characterized by a loss of oligodendrocytes in the central nervous system or Schwann cells in the peripheral nervous system. It can also be detected (e.g. diagnosed) by a decrease in myelinated axons in the nervous system or by a reduction in the levels of oligodendrocyte or Schwann cell markers. In the latter case, exemplary marker proteins of oligodendrocytes or Schwann cells include (but are not limited to): CC1; myelin basic protein (MBP); ceramide galactosyltransferase (CGT); myelin associated glycoprotein (MAG); myelin oligodendrocyte glycoprotein (MOG); oligodendrocyte-myelin glycoprotein (OMG); cyclic nucleotide phosphodiesterase (CNP); NOGO; myelin protein zero (MPZ); peripheral myelin protein 22 (PMP22); protein 2 (P2); galactocerebroside (GalC); sulfatide and proteolipid protein (PLP).

Other methods of diagnosing a disorder of central myelin include MRI of brain and spinal cord to look for altered signal arising from white matter tracts, or the measurement of evoked potentials to look for slower conduction of nerve impulses along white matter tracts. Methods of diagnosing a peripheral disorder of myelin include nerve conduction studies to detect slowed nerve conduction velocities.

Thus, the invention finds particular application the treatment or prophylaxis of subjects having T2 hyperintensity of white matter tracts as revealed by brain MRI and subjects having a peripheral conduction velocity of the median nerve of less than 35 m/s. In both of these subject groups, myelin dysfunction/abnormality is implied and stimulation of myelination by administration of the compounds of the invention may be indicated.

Demyelinating diseases are sporadic or acquired (although genetic factors may play some role) and are characterized by the destruction of biochemically normal myelin, or of the cells that produce it, by inflammatory processes. For example, destruction or functional impairment of myelin-producing Schwann/oligodendrocytes leads to loss of biochemically normal myelin, an example being progressive multifocal leukoencephalopathy (PML) (caused by JC papovavirus infection of oligodendrocytes). The involvement of inflammatory cells directed against specific antigenic myelin components (which may be partitioned between CNS and PNS) means that most demyelinating diseases (unlike the dysmyelinating diseases—see below) involve CNS or PNS exclusively.

The present invention finds broad application in the treatment or prophylaxis of all demyelinating diseases irrespective of aetiology, including for example disease arising from pathogens (such as bacteria, viruses and prions); toxic substances or the accumulation of toxic metabolites in the body (e.g. central pontine myclinolysis and vitamin deficiencies); physical trauma (such as spinal cord injury); and genetic disorders (including the various leukodystrophies discussed in detail below,), tumours in the central nervous system and multiple sclerosis (including in particular the demyelinating component of multiple sclerosis associated with secondary progressive, primary progressive and progressive-relapsing multiple sclerosis).

Exemplary demyelinating diseases which may be treated according to the invention include multiple sclerosis (MS), progressive multifocal leukoencephalopathy (PML), optic neuritis, demyelinating encephalomyelitis (post viral and post vaccinal), central pontine myelolysis (CPM), leukodystrophies; Cockayne syndrome, Van der Knapp syndrome; Guillain-Barre Syndrome (GBS); chronic inflammatory demyelinating polyneuropathy (CIDP), multifocal motor neuropathy (MMN), optic neuritis, transverse myelitis, HMSN types 1 and 3, neuromyelitis optica (NMO), Balo concentric sclerosis, Schilder's diffuse sclerosis and Marburg multiple sclerosis.

### Dysmyelinating Diseases

The invention finds broad application in the treatment or prophylaxis of all dysmyelinating (sometimes referred to as dysmyelinative) diseases, either of the CNS or PNS alone or (more commonly) dysmyelinating diseases in which both the CNS and PNS are affected.

The dysmyelinating diseases which may be treated according to the invention are hereditary and characterized by the presence of biochemically abnormal myelin or an abnormality in the myelin-forming Schwann/oligodendrocytes that produce it.

The invention finds application in the treatment or prophylaxis of all leukodystrophies. Leukodystrophy defines a class of dysmyelinating diseases characterized by progressive degeneration of the white matter of the brain as a result of a defect in a gene involved in the production or metabolism of a specific myelin component. This class includes diseases such as Pelizaeus-Merzbacher Disease (PMD) and Alexander disease).

Specific leukodystrophies that may be treated according to the invention include: 18q syndrome with deficiency of myelin basic protein; adrenoleukodystrophy (ALD); adrenomyeloneuropathy (AMN); Aicardi-Goutiere's syndrome; Alexander disease; autosomal dominant diffuse leukoencephalopathy with neuroaxonal spheroids; autosomal dominant late-onset leukoencephalopathy; childhood ataxia with diffuse CNS hypomyelination (CACH or Vanishing White Matter Disease); Canavan disease; cerebrotendinousxanthomatosis (CTX); craniometaphysical dysplasia with leu koencephalopathy; extensive cerebral white matter abnormality without clinical symptoms; familial leukodystrophy with adult onset dementia and abnormal glycolipid storage; globoid cell leukodystrophy (also known as Krabbe disease); hereditary adult onset leukodystrophy simulating chronic progressive multiple sclerosis; lipomembranous osteodysplasia with leukodystrophy (Nasu Disease); metachromatic leukodystrophy (MLD); megalencephalic leukodystrophy with subcortal cysts (MLC); neonatal ALD; neuroaxonal leukoencephalopathy with axonal spheroids; oculodetatoldigital dysplasia with cerebral white matter abnormalities; orthochormatic leukodystrophy with pigmented glia; ovarioleukodystrophy syndrome; Pelizaeus-Merzbacher Disease (PMD); phenylketonuria (PKU); Refsum disease; Sjogren-Larssen syndrome; sudanophilic leukodystrophy; Van der Knaap Syndrome (vacuolating leukodystrophy with subcortal cysts or MLC); vanishing white matter disease (childhood ataxia with diffuse CNS hypomyelination or CACH); X-linked ALD; Zellweger spectrum and Zellweger syndrome.

### Other Indications

The invention finds broad application in all indications in which remyelination or neuroregeneration/neuroprotection is indicated, and so finds application in the treatment of spinal cord injury, traumatic brain injury, post radiation injury, neurological complications of chemotherapy, stroke, acute ischaemic optic neuropathy, vitamin E deficiency, isolated vitamin E deficiency syndrome.

### Dementias

The compounds of the invention find application in the treatment or prophylaxis of dementia. Any form of dementia may be treated according to the invention, including cortical and subcortical dementias. In preferred embodiments, the invention finds application in the treatment or prophylaxis of a dementia selected from:
(a) vascular dementia;
(b) dementia associated with Alzheimer's disease;
(c) dementia associated with Parkinson's disease;
(d) dementia with Lewy bodies;
(e) dementia induced by demyelinating disease;
(f) toxin-induced dementia, for example alcohol-induced dementia;
(g) dementia associated with vitamin deficiency;
(h) frontotemporal dementia;
(i) dementia associated with Huntington's disease; and
(j) dementia associated with amyotrophic lateral sclerosis.

### Autoimmune Diseases and Allergy

The compounds of the invention find application in the treatment or prophylaxis of various autoimmune diseases and allergy.

The immune system is very effective in the healthy human body at resisting the onslaught of invading viruses, bacteria and parasites. Unfortunately, the immune system sometimes goes awry. Intrathymic deletion of developing T cells with high avidity for self-antigens restricts the repertoire of peripheral autoreactive T cells. When this process does not proceed to completion lymphocytes with avidity for self-antigens are generated in the periphery. While it is thought that the resulting overlap in the recognition of self and non-self is necessary for effective immunity, the phenomenon creates the potential for autoimmune disease. In such diseases, lymphocytes with avidity for self-antigens mediate pathological immune responses (typically inflammation and tissue damage).

These misdirected immune responses are referred to as autoimmunity, which can be demonstrated by the presence of auto-antibodies or T lymphocytes reactive with host antigens. Autoimmune diseases can strike any part of the body, and thus symptoms vary widely and diagnosis and treatment are often difficult. At least ten million Americans suffer from the more than eighty illnesses caused by autoimmunity. They appear to be more prevalent in women; about 75% of the patients are women. Autoimmune diseases are among the ten leading causes of death among women in all age groups up to 65. The ten most prevalent autoimmune diseases in the United States in 1996 were Grave's disease, rheumatoid arthritis, Hashimoto's thyroiditis, vitiligo, type I (early onset) diabetes, pernicious aenemia, multiple sclerosis, glomerulonephritis, systemic lupus E (SLE, lupus) and Sjogren syndrome (see Jacobson et al. (1997) Clin Immunol Immunopathol 84: 223-243). Other important autoimmune diseases include scleroderma, psoriasis, ankylosing spondilitis, myasthenia gravis, pemphigus, polymyositis, dermomyositis, uveitis, Guillain-Barre syndrome, Crohn's disease and ulcerative colitis (frequently referred to collectively as inflammatory bowel disease (IBD)). Some autoimmune diseases such as lupus and pemphigus can be life threatening unless properly diagnosed and treated. Chronic autoimmune disorders like rheumatoid arthritis cripple the patient and also create heavy burdens on patients' families. Some types of uveitis may cause blindness.

Ulcerative colitis and Crohn's disease have no medical cure although anti-bodies are being developed for serious cases. Once the diseases begin, they tend to fluctuate between periods of inactivity (remission) and activity (relapse). They affect approximately 500,000 to 2 million people in the United States. In patients with IBD the immune system is abnormally and chronically activated in the absence of any known invader. The continued abnormal activation of the immune system results in chronic inflammation and ulceration. The susceptibility to abnormal activation of the immune system is genetically inherited.

The invention therefore finds broad application in the treatment or prophylaxis of autoimmune diseases (including thyroid diseases (for example Grave's disease and Hashimoto's thyroiditis), rheumatoid arthritis, rheumatic fever, vitiligo, type I (early onset) diabetes, pernicious aenemia, multiple sclerosis, glomerulonephritis, systemic lupus E (SLE, lupus), Sjogren syndrome, scleroderma, psoriasis, ankylosing spondilitis, myasthenia gravis, pemphigus, polymyositis, dermomyositis, uveitis, Guillain-Barre syndrome, autoimmune pulmonary inflammation, autoimmune inflammatory eye disease, Addison's disease, autoimmune male and female infertility and IBD (for example, Crohn's disease and ulcerative colitis).

The invention also finds application in the remodelling of the immune response variegation in patients undergoing immunosuppressive therapies (or therapies in which immunosuppression is a side effect), in patients suffering from Graft-versus-host disease and as an adjunctive therapy in transplantation.

### Allergy

Compounds of the invention find application in the treatment or prophylaxis of allergy. It is well known that genetically predisposed individuals can become sensitised (allergic) to antigens originating from a variety of environmental sources. The allergic reaction occurs when a previously sensitised individual is re-exposed to the same or to a structurally similar or homologous allergen. Thus, as used herein the term allergy is used to define a state of hypersensitivity induced by exposure to a particular antigen (allergen) resulting in harmful and/or uncomfortable immunologic reactions on subsequent exposures to the allergen.

The harmful, uncomfortable and/or undesirable immunologic reactions present in allergy include a wide range of symptoms. Many different organs and tissues may be affected, including the gastrointestinal tract, the skin, the lungs, the nose and the central nervous system. The symptoms may include abdominal pain, abdominal bloating, disturbance of bowel function, vomiting, rashes, skin irritation, wheezing and shortness of breath, nasal running and nasal blockage, headache and mood changes. In severe cases the cardiovascular and respiratory systems are compromised and anaphylactic shock leads in extreme cases to death.

Any allergy may be treated according to the invention, including atopic allergy, allergic rhinitis, allergic conjunctivitis, atopic dermatitis, hypereosinophilia, irritable bowel syndrome, allergen-induced migraine, bacterial allergy, bronchial allergy (asthma), contact allergy (dermatitis), delayed allergy, pollen allergy (hay fever), drug allergy, sting allergy, bite allergy, gastrointestinal or food allergy (including that associated with inflammatory bowel disease, including ulcerative colitis and Crohn's disease) and physical allergy. Physical allergies include cold allergy (cold urticaria or angioedema), heat allergy (cholinergic urticaria) and photosensitivity.

### Asthma

Compounds of the invention find application in the treatment or prophylaxis of asthma.

There is considerable interest in new approaches to treat asthma. The prevalence of allergic diseases, including asthma, has increased markedly in the past three decades but there are few new drugs to combat them. There is still a reliance on glucocorticoids and bronchodilators which, although effective, require frequent administration and are associated with long-term side-effects. Furthermore, corticosteroids that block inflammatory cells do not alter the prevailing Th2 immune response in sensitized individuals. Therefore, longer-lasting treatments afforded by manipulating the innate immune system would provide a great advance.

### Treatment of Neoplasia

The compounds of the invention find general application in the treatment or prophylaxis of any neoplasia, including proliferative disorders, benign, pre-cancerous and malignant neoplasia, hyperplasia, metaplasia and dysplasia. Particularly preferred in such applications are compounds of the invention which modulate CSC function.

The invention therefore finds application in the treatment or prophylaxis of proliferative disorders which include, but are not limited to cancer, cancer metastasis, smooth muscle cell proliferation, systemic sclerosis, cirrhosis of the liver, adult respiratory distress syndrome, idiopathic cardiomyopathy, lupus erythematosus, retinopathy (e.g. diabetic retinopathy), cardiac hyperplasia, benign prostatic hyperplasia, ovarian cysts, pulmonary fibrosis, endometriosis, fibromatosis, haematomas, lymphangiomatosis, sarcoidosis and desmoid tumours. Neoplasia involving smooth muscle cell proliferation include hyperproliferation of cells in the vasculature (e.g. intimal smooth muscle cell hyperplasia, restenosis and vascular occlusion, including in particular stenosis following biologically- or mechanically-mediated vascular injury, such as angioplasty). Moreover, intimal smooth muscle cell hyperplasia can include hyperplasia in smooth muscle other than the vasculature (e.g. blockage of the bile duct, bronchial airways and in the kidneys of patients with renal interstitial fibrosis). Non-cancerous proliferative disorders also include hyperproliferation of cells in the skin such as psoriasis and its varied clinical forms, Reiter's syndrome, pityriasis rubra pilaris and hyperproliferative variants of disorders of keratinization (including actinic keratosis, senile keratosis and scleroderma).

Particularly preferred is the treatment or prophylaxis of malignant neoplasia (cancer).

The cancers to be treated according to the invention may be of any stage or grade. For example, the compounds of the invention may be used to treat cancers staged by reference to tumour size (T), the degree of regional spread or node involvement (N) and distant metastasis (M) as follows: Carcinoma in situ (limited to surface cells), T1, T2, T3 and T4 (increasing tumour size and involvement, from very localised tumour with no remote metastases, to locally limited extension with or without minimal node satellite extension and with no remote metastases to locally advanced extension with or without major node satellite extension and with no remote metastases); NO (no lymph node involvement), N1, N2, N3 and N4 (increasing degrees of lymph node involvement); MO (no evidence of distant metastases) and M1 (evidence of distant metastases).

The compounds of the invention may be used to treat cancers of any grade. Grading involves examining tumour cells that have been obtained through biopsy under a microscope. The abnormality of the cells determines the grade of the cancer. Increasing abnormality increases the grade (from 1 to 4). Cells that are well differentiated closely resemble mature, specialized cells. Cells that are undifferentiated are highly abnormal, that is, immature and primitive.

The compounds of the invention may be used to treat cancers selected from: (a) Grade 1 (cells slightly abnormal and well differentiated); (b) Grade 2 (cells more abnormal and moderately differentiated); (c) Grade 3 (cells very abnormal and poorly differentiated and (d) Grade 4 (cells immature and undifferentiated).

The compounds of the invention may be used to treat cancers in adults, children or infants. They also find application in the treatment or prophylaxis of drug-resistant cancers.

The invention finds application in the treatment or prophylaxis of any cancer, including those selected from the following major groupings: (a) carcinoma; (b) blastoma; (c) leukemia; (d) lymphoma; (e) myeloma; (f) sarcoma and (g) cancers of mixed type. These are discussed in more detail below.

### Carcinoma

Carcinoma refers to a malignant neoplasm of epithelial origin or cancer of the internal or external lining of the body. Carcinomas, malignancies of epithelial tissue, account for 80 to 90 percent of all cancer cases. Epithelial tissue is found throughout the body. It is present in the skin, as well as the covering and lining of organs and internal passageways, such as the gastrointestinal tract.

Carcinomas are divided into two major subtypes: adenocarcinoma, which develops in an organ or gland, and squamous cell carcinoma, which originates in the squamous epithelium. The invention finds application in the treatment or prophylaxis of all carcinomas, including adenocarcinomas and squamous cell carcinomas, as described below.

Adenocarcinomas generally occur in mucus membranes and are first seen as a thickened plaque-like white mucosa. They often spread easily through the soft tissue where they occur. Squamous cell carcinomas occur in many areas of the body.

Most carcinomas affect organs or glands capable of secretion, such as the mammary glands, lungs, colon, prostate or bladder.

The invention therefore finds application in the treatment or prophylaxis of various carcinomas, for example a carcinoma of the bladder, breast (e.g. primary breast tumours, node-negative breast cancer, invasive duct adenocarcinomas of the breast and non-endometrioid breast cancers), colon (e.g. colorectal carcinomas such as colon adenocarcinoma and colon adenoma), kidney, epidermis (e.g. malignant melanoma), liver, lung (e.g. adenocarcinoma, adrenocortical, nasopharyngeal, small cell lung cancer and non-small cell lung carcinomas), oesophagus, gall bladder, ovary, pancreas (e.g. exocrine pancreatic carcinoma), stomach, cervix, thyroid, prostate, gastrointestinal system (e.g. gastrointestinal stromal tumours) or skin (e.g. squamous cell carcinoma).

In preferred embodiments the carcinoma treated according to the invention is selected from carcinomas of: salivary glands; colon; rectum; appendix; lung; thymus; breast; cervix uteri; bladder and eye.

### Blastoma

The invention finds application in the treatment or prophylaxis of all blastomas, including hepatoblastomas (e.g. nephroblastomas, nonepithelial renal tumours, rhabdoid renal tumour, kidney sarcomas and pPNET of the kidney), medulloblastomas, pancreatoblastomas, pulmonary blastoma, pleuropulmonary blastoma, neuroblastomas (including peripheral nervous cell tumours in general as well as ganglioneuroblastoma and retinoblastomas).

### Leukemias, Myeloproliferative Diseases, and Myelodysplastic Diseases

The invention finds application in the treatment or prophylaxis of all leukemias, myeloproliferative diseases and myelodysplastic diseases, including: lymphoid leukemias (for example precursor cell leukemias, mature B-cell leukemias, mature T-cell leukemias and NK cell leukemias); acute myeloid leukemias; chronic myeloproliferative diseases; myelodysplastic syndrome and other myeloproliferative diseases.

The invention therefore finds application in the treatment or prophylaxis of various leukemias, including lymphatic, lymphocytic, or lymphoblastic leukemia (malignancy of the lymphoid and lymphocytic blood cell series) and polycythemia vera or erythremia (malignancy of various blood cell products, but with red cells predominating).

### Lymphomas and Reticuloendothelial Neoplasms

Lymphomas develop in the glands or nodes of the lymphatic system, a network of vessels, nodes, and organs (specifically the spleen, tonsils, and thymus) that purify bodily fluids and produce infection-fighting white blood cells, or lymphocytes. Unlike the leukemias which are sometimes called “liquid cancers,” lymphomas are “solid cancers” Lymphomas may also occur in specific organs such as the stomach, breast or brain. These lymphomas are referred to as extranodal lymphomas. The lymphomas are subclassified into two categories: Hodgkin lymphoma and Non-Hodgkin lymphoma. The presence of Reed-Sternberg cells in Hodgkin lymphoma diagnostically distinguishes Hodgkin lymphoma from Non-Hodgkin lymphoma.

The invention finds application in the treatment or prophylaxis of all such lymphomas and reticuloendothelial neoplasms, including: (a) Hodgkin lymphomas; (b) Non-Hodgkin lymphomas (for example precursor cell lymphomas, mature B-cell lymphomas, mature T-cell lymphomas and NK-cell lymphomas; (c) Burkitt lymphoma and (d) other lymphoreticular neoplasms, including mantle cell lymphoma.

The invention therefore finds application in the treatment or prophylaxis of a wide range of lymphomas, including for example tumours of the glands or nodes of the lymphatic system (including the spleen, tonsils, and thymus) and extranodal lymphomas of the stomach, breast and brain.

### Myeloma (Multiple Myeloma or Myelomatosis)

Myeloma is cancer that originates in the plasma cells of bone marrow.

The invention therefore finds application in the treatment or prophylaxis of hematopoieitic tumours and haematological malignancies, including those of lymphoid lineage (e.g. leukaemia, acute lymphocytic leukaemia, chronic lymphocytic leukaemia, B-cell lymphoma (such as diffuse large B cell lymphoma), T-cell lymphoma, Hodgkin's lymphoma, non-Hodgkin's lymphoma (the presence of Reed-Sternberg cells in Hodgkin lymphoma distinguishes Hodgkin lymphoma from Non-Hodgkin lymphoma), hairy cell lymphoma and Burkitt's lymphoma) as well as hematopoieitic tumours of myeloid lineage (for example acute myeloid leukaemia, chronic myeloid leukaemias, myelogenous leukaemias and Imatinib sensitive and refractory chronic myelogenous leukaemias, myelodysplastic syndrome, Bortezomib sensitive and refractory multiple myeloma, myeloproliferative disease or promyelocytic leukaemia and thyroid follicular cancer).

### Sarcoma

The invention finds application in the treatment or prophylaxis of all sarcomas. Sarcoma refers to cancer that originates in supportive and connective tissues such as bones, tendons, cartilage, muscle and fat. Generally occurring in young adults, the most common sarcoma often develops as a painful mass on the bone. Sarcoma tumours usually resemble the tissue in which they grow.

Exemplary sarcomas for treatment or prophylaxis according to the invention include osteosarcoma (or osteogenic sarcoma); chondrosarcoma; leiomyosarcoma (smooth muscle); rhabdomyosarcoma (skeletal muscle); mesothelial sarcoma or mesothelioma (membranous lining of body cavities); fibrosarcoma (fibrous tissue); angiosarcoma or hemangioendothelioma (blood vessels); liposarcoma; glioma; astrocytoma; myxosarcoma (primitive embryonic connective tissue) and mesenchymous or mixed mesodermal tumour (mixed connective tissue types).

Fibrosarcomas include peripheral nerve sheath tumours and other fibrous neoplasms, for example fibroblastic and myofibroblastic tumours, nerve sheath tumours and other fibromatous neoplasms. Also included is Kaposi sarcoma.

Also included are soft tissue sarcomas, for example Ewing tumour and Askin tumour of soft tissue, pPNET of soft tissue, extrarenal rhabdoid tumour; fibrohistiocytic tumours; synovial sarcomas; osseous and chondromatous neoplasms of soft tissue and alveolar soft parts sarcoma.

Osteosarcomas (malignant bone tumours) include: malignant fibrous neoplasms of bone; malignant chordomas and odontogenic malignant tumours. Gliomas include oligodendrogliomas, mixed and unspecified gliomas and neuroepithelial glial tumours.

### Mixed Types

The invention finds application in the treatment or prophylaxis of cancers of the mixed type, including for example adenosquamous carcinoma, mixed mesodermal tumour, carcinosarcoma and teratocarcinoma.

The invention therefore finds application in the treatment or prophylaxis of various CNS, PNS and miscellaneous intracranial and intraspinal neoplasms, including: astrocytoma, neuroblastoma, glioma, schwannoma, ependymomas and choroid plexus tumour (for example ependymomas and choroid plexus tumours); intracranial and intraspinal embryonal tumours (for example medulloblastomas, primitive neuroectodermal tumour (PNET), medulloepithelioma, atypical teratoid/rhabdoid tumour and other intracranial and intraspinal neoplasms (for example pituitary adenomas and carcinomas, tumours of the sellar region (craniopharyngiomas), pineal parenchymal tumours, neuronal and mixed neuronal-glial tumours, meningiomas and intracranial and intraspinal neoplasms in general).

Thus, the invention finds particular application in the treatment or prophylaxis of: intracranial and intraspinal germ cell tumours; intracranial and intraspinal germinomas; intracranial and intraspinal teratomas; intracranial and intraspinal embryonal carcinomas; intracranial and intraspinal yolk sac tumour; intracranial and intraspinal choriocarcinoma and intracranial and intraspinal tumours of mixed forms.

The invention also finds application in the treatment or prophylaxis of various germ cell tumours, trophoblastic tumours and neoplasms of the gonads. Thus, the invention finds application in the treatment or prophylaxis of malignant extracranial and extragonadal germ cell tumours including, for example, malignant germinomas of extracranial and extragonadal sites, malignant teratomas of extracranial and extragonadal sites, embryonal carcinomas of extracranial and extragonadal sites, yolk sac tumour of extracranial and extragonadal sites; choriocarcinomas of extracranial and extragonadal sites and malignant mixed germ cell tumours of extracranial and extragonadal sites in general. The invention also finds application in the treatment or prophylaxis of malignant gonadal germ cell tumours, including for example malignant gonadal germinomas, seminomas, malignant gonadal teratomas, gonadal embryonal carcinomas, gonadal yolk sac tumour, gonadal choriocarcinoma, malignant gonadal tumours of mixed forms and malignant gonadal gonadoblastoma.

The invention also finds application in the treatment or prophylaxis of various cancers associated with neoplastic cells having a particular gene expression profile/genotype, as described below:

### C-Kit Associated Neoplasia

CD117, also called KIT, C-kit receptor or c-Kit, is a cytokine receptor expressed on the surface of hematopoietic stem cells. Altered forms of this receptor are associated with certain types of neoplastic cells and the invention finds application in the treatment or prophylaxis of neoplasia involving neoplastic cells having an altered c-kit receptor.

Expression of c-kit is frequently observed in acute myelocytic leukemia (AML), acute lymphocytic leukemia (ALL) and chronic myelogenous leukemia (CML). Constitutive c-kit activation also appears to be important for gastrointestinal stromal tumours (GISTs). GISTs are the most common mesenchymal tumours of the digestive system.

Male germ cell tumours have been histologically categorized into seminomas, which retain germ cell characteristics, and nonseminomas which can display characteristics of embryonal differentiation. These tumours express both c-kit and SCF and an autocrine loop may contribute to the tumourigenesis.

Glioblastoma and astrocytoma arise from neoplastic transformation of astrocytes and expression of c-kit has been observed in glioblastoma cell lines and tissues.

### Bcr-Abl Associated Neoplasia

The Philadelphia chromosome which generates the fusion protein Bcr-Abl is associated with the bulk of chronic myelogenous leukemia (CML) patients (more than 95%), 10-25% of acute lymphocytic leukemia (ALL) patients and 2-3% of acute myelogenous leukemias

(AML). In addition, Bcr-Abl is a factor in a variety of other hematological malignancies, including granulocytic hyperplasia resembling CML, myelomonocytic leukemia, lymphomas, and erythroid leukemia.

### FLT3 Associated Neoplasia

FLT3 associated neoplasias are cancers in which inappropriate FLT3 activity has been implicated as a contributing factor. FLT3 associated cancers include hematologic malignancies such as leukemia and lymphoma. In some embodiments FLT3 associated cancers include acute myelogenous leukemia (AML), B-precursor cell acute lymphoblastic leukemia, myelodysplastic leukemia, T-cell acute lymphoblastic leukemia, mixed lineage leukemia (MLL) and chronic myelogenous leukemia (CML).

### EGFR Associated Neoplasia

EGFR associated cancers are cancers in which inappropriate EGFR activity (e.g. overexpression of EGFR or mutation of EGFR which causes constitutive tyrosine kinase activity) has been implicated as a contributing factor.

The invention therefore finds application in the treatment or prophylaxis of neoplasia associated with neoplasms exhibiting inappropriate EGFR activity, including EGFR associated neuroblastoma, intestine carcinoma (for example rectum carcinoma, colon carcinoma, familiary adenomatous polyposis carcinoma and hereditary non-polyposis colorectal cancer), oesophageal carcinoma, labial carcinoma, larynx carcinoma, hypopharynx carcinoma, tong carcinoma, salivary gland carcinoma, gastric carcinoma, adenocarcinoma, medullary thyroidea carcinoma, papillary thyroidea carcinoma, renal carcinoma, kidney parenchym carcinoma, ovarian carcinoma, cervix carcinoma, uterine corpus carcinoma, endometrium carcinoma, chorion carcinoma, pancreatic carcinoma, prostate carcinoma, testis carcinoma, breast carcinoma, urinary carcinoma, melanoma, brain tumours such as glioblastoma, astrocytoma, meningioma, medulloblastoma and peripheral neuroectodermal tumours, Hodgkin lymphoma, non-Hodgkin lymphoma, Burkitt lymphoma, acute lymphatic leukemia (ALL), chronic lymphocytic leukemia (CLL), acute myeloid leukemia (AML), chronic myeloid leukemia (CML), adult T-cell leukemia lymphoma, hepatocellular carcinoma, gall bladder carcinoma, bronchial carcinoma, small cell lung carcinoma, non-small cell lung carcinoma, multiple myeloma, basalioma, teratoma, retinoblastoma, choroidea melanoma, seminoma, rhabdomyo sarcoma, craniopharyngeoma, osteosarcoma, chondrosarcoma, myosarcoma, liposarcoma, fibrosarcoma, Ewing sarcoma and plasmocytoma.

EGFR appears to have an important role in the development of human brain tumours: the amplification of the EGFR gene in glioblastoma multiforme tumours is one of the most consistent genetic alterations known, with EGFR being overexpressed in approximately 40% of malignant gliomas and EGFRvIII mutation being found in about 50% of all glioblastomas.

In addition to gliomas, abnormal EGFR expression has also been reported in a number of squamous epidermoid cancers and breast cancers. Over-expression of EGFR may be associated with a poorer prognosis relative to tumours that do not over-express EGFR.

### Immunomodulation

### Induction of Immunosuppression

The compounds of the invention may be used as immune suppressants to alleviate, control or modify states in which the immune system is overactive. Thus, the compounds of the invention may find application in the treatment or prophylaxis of various autoimmune diseases, the treatment or management of transplant rejection, graft versus host diseases (GVHD) and allergy.

The invention therefore finds broad application in the treatment or prophylaxis of autoimmune diseases (including thyroid diseases (for example Grave's disease and Hashimoto's thyroiditis), rheumatoid arthritis, rheumatic fever, vitiligo, type I (early onset) diabetes, pernicious aenemia, multiple sclerosis, glomerulonephritis, systemic lupus E (SLE, lupus), Sjogren syndrome, scleroderma, psoriasis, ankylosing spondilitis, myasthenia gravis, pemphigus, polymyositis, dermomyositis, uveitis, Guillain-Barre syndrome, autoimmune pulmonary inflammation, autoimmune inflammatory eye disease, Addison's disease, autoimmune male and female infertility and IBD (for example, Crohn's disease and ulcerative colitis).

Any allergy may be treated according to the invention, including atopic allergy, allergic rhinitis, allergic conjunctivitis, atopic dermatitis, hypereosinophilia, irritable bowel syndrome, allergen-induced migraine, bacterial allergy, bronchial allergy (asthma), contact allergy (dermatitis), delayed allergy, pollen allergy (hay fever), drug allergy, sting allergy, bite allergy, gastrointestinal or food allergy (including that associated with inflammatory bowel disease, including ulcerative colitis and Crohn's disease) and physical allergy. Physical allergies include cold allergy (cold urticaria or angioedema), heat allergy (cholinergic urticaria) and photosensitivity.

### Alleviation of Immunosuppression

The compounds of the invention may be used as immune stimulants to alleviate, control or modify states in which the immune system is partially or completely suppressed or depressed. Such states may arise from congenital (inherited) conditions, be acquired (e.g. by infection or malignancy) or induced (e.g. deliberately as part of the management of transplants or cancers).

Thus, the compounds of the invention may find application as adjunctive immunomodulators (e.g. immunostimulants) in the treatment or prophylaxis and/or management of various diseases (including certain cancers) or medical interventions (including radiotherapy, immunosupressant therapies (such as the administration of cyclosporine A, azathioprine or immunosuppressant radiotherapies), chemotherapy and cytotoxic drug administration (for example the administration of ricin, cyclophosphamide, cortisone acetate, vinblastine, vincristine, adriamycin, 6-mercaptopurine, 5-fluorouracil, mitomycin C, chloramphenicol and other steroid-based therapies). They may therefore be used as chemoprotectants in the management of various cancers and infections (including bacterial and viral infections, e.g. HIV infection) or to induce appropriate and complementary immunotherapeutic activity during conventional immunotherapy.

In particular, the compounds of the invention may find application as immunostimulants in the treatment or prophylaxis or management of microbial infections which are associated with immune-suppressed states, including many viral infections (including HIV infection in AIDS) and in other situations where a patient has been immunocompromised (e.g. following infection with hepatitis C, or other viruses or infectious agents including bacteria, fungi, and parasites, in patients undergoing bone marrow transplants, and in patients with chemical or tumour-induced immune suppression).

Other diseases or disorders which may give rise to an immunosupressed state treatable according to the invention include: ataxia-telangiectasia; DiGeorge syndrome; Chediak-Higashi syndrome; Job syndrome; leukocyte adhesion defects; panhypogammaglobulinemia (e.g. associated with Bruton disease or congenital agammaglobulinemia); selective deficiency of IgA; combined immunodeficiency disease; Wiscott-Aldrich syndrome and complement deficiencies. It may be associated with organ and/or tissue (e.g. bone marrow) transplantation or grafting, in which applications the compounds of the invention may be used adjunctively as part of an overall treatment regimen including surgery and post-operative management of immune status.

### Cytokine Stimulation

Compounds of the invention may be used to induce, potentiate or activate various cytokines in vivo, including various interleukins (including IL-2 and/or IL-12). Accordingly, the invention finds general application in the treatment or prophylaxis of conditions in which the in vivo induction, potentiation or activation of one or more cytokines is indicated (and optionally in the treatment or prophylaxis of conditions in which the in vivo induction, potentiation or activation of one or more Th1 cytokines (e.g. IL-12) is indicated). Such applications may be employed to stimulate particular elements of the cellular immunity system, including dendritic cells, macrophages (e.g. tissue-specific macrophages), CTL, NK, NKT, B and LAK cells.

Particularly preferred is the use of the compounds of the invention to stimulate the secretion of systemic IL-12 in the treatment or prophylaxis of cancer and cancer metastases

In such applications, the invention may be employed as an adjunct to gene therapies designed to increase the production of endogenous cytokines.

### Pathogen-Mediated Immune System Subversion

Compounds of the invention find application in the treatment or prophylaxis of pathogen-mediated immune system subversion.

Certain microbial compounds produced during infection can arrest DCs in their immature state and/or instruct (or prime) them on maturation to initiate an immune response that is polarized towards a T_{REG} type. The resultant immunological tolerance benefits the pathogen and can permit chronic infection. Many eukaryotic pathogens (including protozoa, helminths, fungi and ectoparasites) can act in this way.

Examples of pathogens which subvert the host immune response by arresting DCs in the immature state or otherwise inhibiting DC activation or function include Plasmodium falciparum (by binding to CD36 and CD52), Mycobacterium spp. (which can bind to DC-SIGN via mannosylated lipoarabinomannan), hepatitis C virus (HCV), herpes simplex virus (HSV), cytomegalovirus, Bacillus anthracis, lymphocytic choriomeningitis virus (LCMV) and lymphocytic choriomeningitis virus (LCMV). Examples of pathogens which instruct or prime DCs to initiate an immune response that is polarized towards a T_{REG} type include Bordatella pertussis and Schistosoma mansoni.

The invention may therefore find application in the treatment of chronic infection or infestation caused by any of the foregoing pathogens.

Certain pathogens may also induce neutropenia. Neutrophils are short lived, phagocytic cells which are important in host resistance to microbial invasion. They play a protective role in challenges by Candida albicans, Salmonella enteric subspecies, Tyhipimurium, Yersinia enterocolitica, Chlamydia trachomatis and Toxoplasma gondii.

Neutropenia (depleted levels of circulating neutrophils) is a risk associated with the above mentioned infections. Evidence has shown that impaired protective acquired immunity is correlated to neutropenia, hence the importance of neutrophil presence during immune responses. During infection with Toxoplasma gondii, for example, neutrophil depletion leads to impaired immunity and lethal systemic pathology (Bennouna et al., 2003).

Thus, the invention finds application in the treatment or prophylaxis of pathogen-mediated neutropenia, for example in the treatment or prophylaxis of the clinical sequelae of infection with a pathogen selected from HIV, Candida albicans, Salmonella enteric subspecies, Tyhipimurium, Yersinia enterocolitica, Chlamydia trachomatis and Toxoplasma gondii.

### Energy Utilization Diseases

Energy utilization diseases encompass a wide range of diseases and include, for example, disorders of homeostasis, metabolic diseases, dysfunction of sugar metabolism and appetite disorders.

Glycosylation has an important role in regulating properties of proteins and is associated with many diseases. Itoh, N. et al., 2007 (Am J Physiol Endocrinol Metab 293: E1069-E1077) have reported the serum N-glycan profile in human subjects with type 2 diabetes and found an increased amount of a biantennary N-glycan that had an α1,6-fucose with a bisecting N-acetylglucosamine. Copeland, R. J. et al., 2008 (Am J Physiol Endocrinol Metab 295: E17-E28) have reviewed the importance of O-linked N-acetylglucosamine in diabetes and concluded that there is a strong positive correlation between GlcNAcylation and the development of insulin resistance. O-linked-β-N-acetylglucosamine (O-GlcNAc) is a dynamic posttranslational modification that, analogous to phosphorylation, cycles on and off serine and/or threonine hydroxyl groups. Cycling of O-GlcNAc is regulated by the concerted actions of O-GlcNAc transferase and O-GlcNAcase. GlcNAcylation is involved in the aetiology of glucose toxicity and chronic hyperglycemia-induced insulin resistance, a major hallmark of type 2 diabetes. Hexosaminidase activity has been shown to be elevated in the serum of diabetics (e.g. Agardh, C. D. et al., 1982, Acta Med. Scand. 212:39-41).

Examples of energy utilization diseases therefore include insulin resistance, various forms of diabetes, metabolic syndrome, obesity, wasting syndromes (for example, cancer associated cachexia), myopathies, gastrointestinal disease, growth retardation, hypercholesterolemia, atherosclerosis and age-associated metabolic dysfunction.

Energy utilization diseases also include conditions associated with metabolic syndrome, obesity and/or diabetes, including for example hyperglycaemia, glucose intolerance, hyperinsulinaemia, glucosuria, metabolic acidosis, cataracts, diabetic neuropathy, diabetic nephropathy, diabetic retinopathy, macular degeneration, glomerulosclerosis, diabetic cardiomyopathy, insulin resistance, impaired glucose metabolism, arthritis, hypertension, hyperlipidemia, osteoporosis, osteopenia, bone loss, brittle bone syndromes, acute coronary syndrome, infertility, short bowel syndrome, chronic fatigue, eating disorders and intestinal motility dysfunction.

The invention finds broad application in the treatment or prophylaxis of any energy utilization disease. Thus, diseases which may be treated according to the invention include, for example, disorders of homeostasis, metabolic diseases, dysfunction of sugar metabolism and appetite disorders.

In preferred embodiments, the invention finds application in the treatment or prophylaxis of insulin resistance, various forms of diabetes, metabolic syndrome, obesity, wasting syndromes (for example, cancer associated cachexia), myopathies, gastrointestinal disease, growth retardation, hypercholesterolemia, atherosclerosis and age-associated metabolic dysfunction.

The invention may also be used for the treatment or prophylaxis of conditions associated with metabolic syndrome, obesity and/or diabetes, including for example hyperglycaemia, glucose intolerance, hyperinsulinaemia, glucosuria, metabolic acidosis, cataracts, diabetic neuropathy, diabetic nephropathy, diabetic retinopathy, macular degeneration, glomerulosclerosis, diabetic cardiomyopathy, insulin resistance, impaired glucose metabolism, arthritis, hypertension, hyperlipidemia, osteoporosis, osteopenia, bone loss, brittle bone syndromes, acute coronary syndrome, infertility, short bowel syndrome, chronic fatigue, eating disorders, intestinal motility dysfunction and sugar metabolism dysfunction.

The invention may also be used to suppress appetite.

Particularly preferred is the treatment or prophylaxis of insulin resistance, metabolic syndrome, obesity and diabetes (particularly type 2 diabetes).

### Insulin Resistance, Metabolic Syndrome and Diabetes

Thus, the invention finds application in the treatment or prophylaxis of insulin resistance, metabolic syndrome and diabetes. Insulin resistance is characterized by a reduced action of insulin in skeletal muscle, adipocytes and hepatocytes so that normal amounts of insulin become inadequate to produce a normal insulin response from the cells of these tissues. In adipocytes, insulin resistance results in hydrolysis of stored triglycerides, leading to elevated free fatty acids in the blood plasma. In muscle, insulin resistance reduces glucose uptake while in hepatocytes it reduces glucose storage. In both of the latter cases an elevation of blood glucose concentrations results. High plasma levels of insulin and glucose due to insulin resistance often progresses to metabolic syndrome and type 2 diabetes.

The invention finds application in the treatment or prophylaxis of metabolic syndrome (as herein defined). The disorder is also known as (metabolic) syndrome X, insulin resistance syndrome, Reaven's syndrome and CHAOS.

The invention finds application in the treatment or prophylaxis of diseases associated with metabolic syndrome, including for example: fatty liver (often progressing to non-alcoholic fatty liver disease), polycystic ovarian syndrome, hemochromatosis (iron overload) and acanthosis nigricans (dark skin patches).

The invention finds application in the treatment or prophylaxis of Type 2 diabetes. Type 2 diabetes is a chronic disease that is characterised by persistently elevated blood glucose levels (hyperglycaemia). Insulin resistance together with impaired insulin secretion from the pancreatic β-cells characterizes the disease. The progression of insulin resistance to type 2 diabetes is marked by the development of hyperglycaemia after eating when pancreatic β-cells become unable to produce adequate insulin to maintain normal blood sugar levels (euglycemia)).

The invention also finds application in the treatment or prophylaxis of Type 1 diabetes (or insulin dependent diabetes). Type 1 diabetes is characterized by loss of the insulin-producing beta cells of the islets of Langerhans in the pancreas, leading to a deficiency of insulin. The main cause of this beta cell loss is a T-cell mediated autoimmune attack. There is no known preventative measure that can be taken against type 1 diabetes, which comprises up to 10% of diabetes mellitus cases in North America and Europe. Most affected people are otherwise healthy and of a healthy weight when onset occurs. Sensitivity and responsiveness to insulin are usually normal, especially in the early stages.

Thus, the compounds of the invention may be used in the therapy and prophylaxis of energy utilization diseases selected inter alia from: (a) disorders of homeostasis; (b) metabolic diseases; (c) dysfunction of sugar metabolism; (d) appetite disorders; (e) insulin resistance; (f) diabetes (e.g. type 1 or type 2 diabetes); (g) pre-diabetes; (h) metabolic syndrome; (i) obesity; (j) wasting syndromes (for example, cancer associated cachexia); (k) myopathies; (l) gastrointestinal disease; (m) growth retardation; (n) hypercholesterolemia; (o) atherosclerosis; (p) age-associated metabolic dysfunction; (q) hyperglycaemia; (r) glucose intolerance; (s) hyperinsulinaemia; (t) glucosuria; (u) metabolic acidosis; (v) cataracts; (w) diabetic neuropathy; (x) diabetic nephropathy; (y) diabetic retinopathy; (z) macular degeneration; (aa) glomerulosclerosis; (bb) diabetic cardiomyopathy; (cc) impaired glucose metabolism; (dd) arthritis; (ee) hypertension; (ff) hyperlipidemia; (gg) osteoporosis; (hh) osteopenia; (ii) bone loss; (jj) brittle bone syndromes; (kk) acute coronary syndrome; (ll) infertility; (mm) short bowel syndrome; (nn) chronic fatigue; (oo) eating disorders; (pp) intestinal motility dysfunction; (qq) sugar metabolism dysfunction; (rr) fatty liver; (ss) polycystic ovarian syndrome; (tt) hemochromatosis; and (uu) acanthosis nigricans.

### Cardiovascular Disorders

Compounds of the invention find application in the treatment or prophylaxis of various cardiovascular disorders. The cardiac disorder may be, for example, ischemia; hemorrhage; hypovolemic shock; myocardial infarction; an interventional cardiology procedure; cardiac bypass surgery; fibrinolytic therapy; angioplasty or stent placement.

The invention provides methods of enhancing or elevating levels of protein O-GlcNAc modification in animal subjects, such as, veterinary and human subjects and/or of selectively inhibiting an OGA enzyme in animal subjects, such as veterinary and human subjects.

The invention provides also provides methods of inhibiting phosphorylation of tau peptides, or inhibiting formation of NFTs, in animal subjects, such as veterinary and human subjects.

In some embodiments, the compounds are also useful as a result of other biological activities related to their ability to inhibit the activity of glycoside hydrolase enzymes.

The compounds of the present invention elevate O-GlcNAc levels on O-GlcNAc-modified polypeptides or proteins in vitro specifically via interaction with an OGA enzyme, and are effective in treating conditions which require or respond to inhibition of OGA activity.

### Adjunctive Agents for Use in the Combinations of the Invention

Any of a wide variety of adjunctive agents may be used in the combinations of the invention, including the exemplary adjunctive agents described below.

When used adjunctively, the compounds of the invention may be formulated for use with one or more other drug(s). In particular, the compounds of the invention may be used in combination with one or more adjunctive agent(s) selected from those identified below.

The adjunctive agent may be an agent useful in the treatment of an energy utilization diseases, in particular diabetes (including type 1 diabetes, type 2 diabetes and insulin resistance) and metabolic syndrome (including any disease or disorder associated therewith, for example central obesity and elevated levels of triglycerides). Thus, the adjunctive agent may be selected from:
Anti-atherogenic agents
Anti-hypertensive agents
Anti-diabetic agents
Anti-thrombotic agents
Anti-obesity agents

Preferred anti-atherogenic agents include: (a) HMG-CoA reductase inhibitors (a.k.a. statins, for example atorvastatin, fluvastatin, lovastatin and simvastatin); (b) cholesterol absorption blockers (e.g. ezetimibe); (c) bile-acid sequestrants (e.g. cholestyramine, colestipol and colesevelam); (d) nicotinic acid; (e) peroxisome proliferator-activated receptor α (PPARα) agonists (e.g. fibrates such as bezafibrate, ciprofibrate, clofibrate, gemfibrozil and fenofibrate); (f) dual PPARγ/PPARα agonists and (f) torcetrabib; and (g) squalene synthetase inhibitors.

Preferred anti-hypertensive agents include: (a) diuretics (e.g. furosamide and hydrochlorothiazide); (b) beta-blockers (e.g. propranolol, metaprolol and atenolol); (c) angiotensin converting enzyme (ACE) inhibitors (e.g. captopril and enalapril); (d) angiotensin II receptor antagonists (e.g. losartan, candesartan and olmesartan); (e) dual angiotensin II receptor antagonists/PPARγ agonists (e.g. telmisartan); (f) calcium channel blockers (e.g. nitrendipine, nicardapine, felodipine, verapamil and diltiazem) and (g) alpha-blockers; and (h) imidazoline receptor antagonists (e.g. moxonidine and rilmenidine).

Preferred anti-diabetic agents include: (a) insulin secretagogues; (b) metformins; (c) insulin sensitizers/antihyperglycaemic agents; (d) PPARγ agonists (e.g. thiazolidinones such as rosiglitazone and pioglitazone); (d) glitazones (e.g. thiazolidinediones, such as pioglitazone and rosiglitazone); (e) insulin and insulin mimetics; (f) prandial glucose regulators; (g) selective peroxisome proliferator-activated receptor modulators (SPPARMs); (h) glitazars; (i) incretin mimetics (e.g. glucagon-like peptide analogues and analogues of GLP-1, such as liraglutide, exenatide, GSK716155, AVE0010, R1583 and CJC-1134-PC); (j) 11 β-hydroxysteroid dehydrogenase 1 inhibitors (a.k.a. 11 β HSD inhibitors) (e.g. INCB13739); (k) dipeptidyl peptidase-4 (DPP-4) inhibitors (e.g. sitagliptin, saxagliptin, alogliptin and vildagliptin); (l) inhibitors of low affinity sodium glucose cotransporter (SGLT2) (e.g. sergliflozin, dapagliflozin and remogliflozin etabonate); (m) glucokinase modulators (e.g. R-1511, piragliatin, AZD-1656, AZD-6370, TTP-355, NN-9101, PSN-010 and TTP-399); (n) glycogen phosphorylase inhibitors (e.g. JTT-651, FR-258900, ingliforib and PSN-357) and (o) G protein-coupled receptor 119 (GPR119) agonists (e.g. PSN821 and APD668).

Preferred insulin secretagogues include: (a) sulphonylureas (e.g. glipizide, glibenclamide, gliclazide, glibornuride and glimepiride); (b) meglitinides (e.g. nateglinide, repaglinide and their analogues); and (c) imidazoline insulin secretagogues (e.g. afaroxan and tolbutamide). Preferred anti-thrombotic agents include: (a) aspirin; (b) clopidogrel. Preferred anti-obesity agents include: (a) food absorption inhibitors (e.g. α-glucosidase inhibitors such as acarbose, miglitol and emiglitate); (b) CNS-active agents; (c) energy expenditure promoters (e.g. β₃-adrenoceptor agonists, thyroid hormone receptor β-subtype agonists and peroxisome proliferator-activated receptor-δ (PPARδ agonists) and (d) G protein-coupled receptor 119 (GPR119) agonists (e.g. PSN821 and APD668). Preferred CNS-active anti-obesity agents include: (a) serotonin (5-hydroxytryptamine) agents; (b) D-fenfluramine; (c) sibutramine; (d) leptin-like agents; (e) agonists for melanocortin 3 and 4 receptors; (f) gastrointestinal hormone analogues; (g) peptide YY analogues; (h) ghrelin antagonists; (i) cholecystokinin receptor agonists; (j) somatostatin receptor antagonists (e.g. octreotide); (k) pancreatic amylin and amylin agonists (e.g. pramlintide); (I) cannabinoid receptor antagonists (e.g. rimonabant and SR-147778); (m) antiepilepsy agents (e.g. topiramate and zonisamide) and (n) antidepressants (e.g. bupropion). Preferred food absorption inhibiting anti-obesity agents include orlistat.

Yet other adjunctive agents for use with the compounds of the invention are antiinflammatory agents, for example selected from those identified below:

### COX/LOX Inhibitors

Cycloxygenase-1 and -2 (COX-1 and COX-2 or prostaglandin H synthases 1 and 2) catalyze the conversion of arachidonic acid and oxygen to generate inflammatory prostaglandins such as PGE2, PGD2, PGF2a, and thromboxane. COX-2 is ubiquitously expressed in the brain by neurons, microglia, endothelial cells and oligodendrocytes. Upregulation of COX-2 has been demonstrated in a number of diseases of the CNS which involve an immune-mediated inflammatory response and it appears that it contributes to inflammation, oligodendrocyte degeneration, demyelination and axonal loss in MS and other myelinative diseases. Furthermore, COX-2 expression is dramatically induced in neurons following ischemic injury, in Alzheimer's disease (AD). COX (and in particular COX-2) inhibitors may therefore have therapeutic application in MS and in other myelinative diseases.

COX-1/-2 and 5-LOX are important inflammatory mediators in the pathogenesis of disease models of myelinative disease and inhibition of both COX and LOX by dual COX-LOX inhibitors (such as phenidone) has been suggested to ameliorate autoimmune disorders of the central nervous system (see Moon et al. (2005) Brain Research: 1035 (2), 206-210). Other dual COX-LOX inhibitors suitable for use with the compounds of the invention include licofelone (ML3000), a COX-1/COX-2/5-LOX inhibitor that downregulates CXCR3 ligands (see e.g. Ospelt et al. (2007) Ann Rheum Dis. doi:10.1136/ard.2007.071589).

### Sodium Channel Blockers

Inflammation associated with many myelinative diseases (including MS) may lead to axons being flooded with toxic levels of sodium from the surrounding tissue fluid. This inflammatory process may be prevented by sodium channel blockers. Lamotrigine is currently being evaluated as a treatment for secondary progressive MS.

nNOS Stimulators

The loss of dystrophin leads to breaks in the membrane, and destabilizes neuronal nitric oxide synthase (nNOS), a protein which normally generates nitric oxide (NO). It is thought that at least part of the muscle degeneration observed in DMD patients may result from the reduced production of muscle membrane-associated neuronal nitric oxide synthase. This reduction may lead to impaired regulation of the vasoconstrictor response and eventual muscle damage.

### Nuclear Factor Kappa-B Inhibitors

A preferred class of antiinflammatory agent suitable for use in the combinations of the invention are Nuclear Factor Kappa-B (NF-kB) inhibitors. NF-kB is a major transcription factor modulating the cellular immune, inflammatory and proliferative responses. NF-kB functions in activated macrophages to promote inflammation and muscle necrosis and in skeletal muscle fibers to limit regeneration through the inhibition of muscle progenitor cells. The activation of this factor in DMD contributes to diseases pathology. Thus, NF-kB plays an important role in the progression of muscular dystrophy and the IKK/NF-B signaling pathway is a potential therapeutic target for the treatment of DMD. Inhibitors of NF-kB (for example, IRFI 042, a vitamin E analogue) ameliorate muscle function, decrease serum CK level and muscle necrosis and enhance muscle regeneration. Furthermore, specific inhibition of NF-kB/IKK-mediated signalling has similar benefits.

### TNF-α Antagonists

TNFα is one of the key cytokines that triggers and sustains the inflammation response. In one embodiment of the invention, the adjunctive agent is a TNF-α antagonist (e.g. infliximab).

Preferences and specific embodiments: Preferred TNF-α antagonists for use according to the invention include infliximab (Remicade™), a chimeric monoclonal antibody comprising murine VK and VH domains and human constant Fc domains. The drug blocks the action of TNFα by binding to it and preventing it from signaling the receptors for TNFα on the surface of cells. Another preferred TNF-α antagonists for use according to the invention is adalimumab (Humira™). Adalimumab is a fully human monoclonal antibody. Another preferred TNF-α antagonists for use according to the invention is etanercept (Enbrel™) Etanercept is a dimeric fusion protein comprising soluble human TNF receptor linked to an Fc portion of an IgG1. It is a large molecule that binds to and so blocks the action of TNFα. Etanercept mimics the inhibitory effects of naturally occurring soluble TNF receptors, but as a fusion protein it has a greatly extended half-life in the bloodstream and therefore a more profound and long-lasting inhibitory effect. Enbrel is marketed as a lyophylized powder in 25 mg vials which must be reconstituted with a diluent and then injected subcutaneously, typically by the patient at home.

Another preferred TNF-α antagonist for use according to the invention is pentoxifylline (Trental™), chemical name 1-(5-oxohexyl)-3,7-dimethylxanthine. The usual dosage in controlled-release tablet form is one tablet (400 mg) three times a day with meals.

### Posology:

Remicade is administered by intravenous infusion, typically at 2-month intervals. The recommended dose is 3 mg/kg given as an intravenous infusion followed with additional similar doses at 2 and 6 weeks after the first infusion then every 8 weeks thereafter. For patients who have an incomplete response, consideration may be given to adjusting the dose up to 10 mg/kg or treating as often as every 4 weeks. Humira is marketed in both preloaded 0.8 ml syringes and also in preloaded pen devices, both injected subcutaneously, typically by the patient at home. Etanercept can be administered at a dose of 25 mg (twice weekly) or 50 mg (once weekly).

### Ciclosporin

In one embodiment of the invention, the antinflammatory agent is ciclosporin. Ciclosporin A, the main form of the drug, is a cyclic nonribosomal peptide of 11 amino acids produced by the fungus Tolypocladium inflatum. Ciclosporin is thought to bind to the cytosolic protein cyclophilin (immunophilin) of immunocompetent lymphocytes (especially T-lymphocytes). This complex of ciclosporin and cyclophylin inhibits calcineurin, which under normal circumstances is responsible for activating the transcription of interleukin-2. It also inhibits lymphokine production and interleukin release and therefore leads to a reduced function of effector T-cells. It does not affect cytostatic activity. It has also an effect on mitochondria, preventing the mitochondrial PT pore from opening, thus inhibiting cytochrome c release (a potent apoptotic stimulation factor). Ciclosporin may be administered at a dose of 1-10 mg/kg/day.

### Corticosteroids

In one embodiment of the invention, the adjunctive agent is a corticosteroid.

### Definition and Biological Activities:

The term “corticosteroid” as used herein refers to any of several steroid hormones secreted by the cortex of the adrenal glands and which are involved in one or more of the following physiological processes: stress response, immune response and regulation of inflammation, carbohydrate metabolism, protein catabolism and blood electrolyte levels. The term also includes synthetic analogues which share the aforementioned properties. Corticosteroids include glucocorticoids and mineralocorticoids. Glucocorticoids control carbohydrate, fat and protein metabolism and are anti-inflammatory. Mineralocorticoids control electrolyte and water levels, mainly by promoting sodium retention in the kidney. Some corticosteroids have dual glucocorticoid and mineralocorticoid activities. For example, prednisone (see below) and its derivatives have some mineralocorticoid action in addition to a glucocorticoid effect. The precise cellular mechanism(s) by which corticosteroids produce antidystrophic effects are not yet known. A multifactorial mechanism is likely and the effects of corticosteroids probably involve a reduction of inflammation, suppression of the immune system, improvement in calcium homeostasis, upregulation of the expression of compensatory proteins and an increase in myoblast proliferation.

### Problems:

The use of corticosteroids is associated with side effects which vary from person to person and on the dosage of the regime used, but they can be severe. The most common side effects are weight gain and mood changes. Weight gain (and attendant changes in muscle activity and use) can abrogate some of the benefits of treatment. Long-term use may lead to growth suppression, cataracts, osteoporosis and muscle atrophy (affecting the same proximal muscles affected in DMD and BMD). These side effects may limit the long-term effectiveness of corticosteroid therapy. Other side effects include hypertension, diabetes, skin atrophy, poor wound healing and immunosuppression. Deflazacort was evaluated in the hope that it would have fewer side effects than prednisone.

### Preferences and Specific Embodiments

Examples include methylprednisolone, prednisone, prednisolone, dexamethasone and adreno-corticotrophic hormone (ACTH or corticotropin), though use of the latter has been largely replaced by synthetic corticosteroids. Preferred are glucocorticoids (or corticosteroids having dual glucocorticoid/minerlocorticoid activity). Synthetic corticosteroids are preferred. In one embodiment, the corticosteroid is prednisone (prodrug) or prednisolone (liver metabolite of prednisone and active drug). In another embodiment, the corticosteroid is deflazacort. Deflazacort is an oxazoline analogue of prednisone. Other synthetic corticosteroids suitable for use in the combinations of the invention include one or more corticosteroids selected from: alclometasone, amcinonide, beclomethasone (including beclomethasone dipropionate), betamethasone, budesonide, ciclesonide, clobetasol, clobetasone, clocortolone, cloprednol, cortivazol, deoxycorticosterone, desonide, desoximetasone, dexamethasone, diflorasone, diflucortolone, difluprednate, fluclorolone, fludrocortisone, fludroxycortide, flumetasone, flunisolide, fluocinolone acetonide, fluocinonide, fluocortin, fluocortolone, fluorometholone, fluperolone, fluprednidene, fluticasone, formocortal, halcinonide, halometasone, hydrocortisone aceponate, hydrocortisone buteprate, hydrocortisone butyrate, loteprednol, medrysone, meprednisone, methylprednisolone, methylprednisolone aceponate, mometasone furoate, paramethasone, prednicarbate, prednylidene, rimexolone, tixocortol, triamcinolone and ulobetasol (or combinations and/or derivatives (e.g. pharmaceutically acceptable salts) of one or more of the foregoing). Suitable endogenous corticosteroids for use in the combinations of the invention include include one or more corticosteroids selected from aldosterone, cortisone, hydrocortisone/cortisol and desoxycortone (or combinations and/or derivatives (e.g. pharmaceutically acceptable salts) of one or more of the foregoing).

### Posology:

Prednisone may be administered daily in dosages ranging from 0.3 to 1.5 mg/kg (typically 0.7 mg/kg). Some patients respond better to 2.5 mg/kg every other day. Deflazacort has an estimated dosage equivalency of 1:1.3 compared with prednisone, though biological equivalence between deflazacort and prednisone also depends on the specific actions under examination. Corticosterods (including delazacort and prednisone) are usually taken orally but can be delivered by intramuscular injection.

Examples of other suitable adjunctive agents include anticholinergics, phosphodiesterase inhibitors (especially those suitable for inhalation and including theophylline and other hypoxanthines), long-acting β adrenergic agonists, glucocorticoids, non-steroidal antiinflammatory drugs (NSAIDS), cromolyn sulphate and leukotriene inhibitors.

Other combinations find particular utility in the treatment of multiple sclerosis, and in particular in the treatment of: (a) secondary progressive multiple sclerosis; (b) primary progressive multiple sclerosis; and (c) progressive relapsing multiple sclerosis.

Thus, further preferred adjunctive agents include agents used in the treatment of multiple sclerosis, for example selected from those agents described below.
1. Myelin repair agents;
2. Antiinflammatory agents;
3. Interferon-β;
4. Corticosteroids/ACTH;
5. Immunomodulators;
6. Anti-excitotoxics;
7. Combinations of two or more of the foregoing classes.

### 1. Myelin Repair Agents

### (a) Immunoglobulins

Suitable immunoglobulins for use as auxiliary myelin repair agents in combination with the compounds of the invention include anti-myelin and anti-oligodendrocyte (Asakura et al. (1996) J. Neurosci. Res. 43: 273-281) antibodies. Other suitable immunoglobulins for use as promyelinative agents in combination with the compounds of the invention are anti-LINGO-1 antibodies. LINGO-1 appears to act as a molecular switch that controls myelin production and acts to prevent myelin repair after injury. Blocking it can promote spinal cord remyelination and axonal integrity (Mi et al. (2007) Nature Medicine 13: 1228-1233).

### (b) Growth Factors

Suitable growth factors for use as auxiliary myelin repair agents in combination with the compounds of the invention include insulin-like growth factor (IGF) I and platelet-derived growth factor (PDGF).

### (c) Glial Cells

Remyelination may be achieved by grafting various classes of promylenigenic cells, including oligodendrocytes (and their precursors), stem cells, immortalized cell lines, xenografts, Schwann cells and olfactory glia (see Scolding (1999) Phil. Trans. R. Soc. B. 354: 1711-1720 for a review).

### 2. Antiinflammatory Agents

A list of preferred adjunctive antiinflammatory agents is set out above.

### 3. Interferon-β

IFN-β has numerous biological properties and its mechanism of action is still poorly understood. However, it is thought to act primarily via immunomodulation. Two formulations of interferon β-1a and one of interferon β-1b have been approved by regulatory authorities for the treatment of multiple sclerosis. IFN-β-1a is identical to the natural IFN-β whereas IFN-β-1b differs by two amino acids and is not glycosylated.

Notwithstanding these differences, IFN-β-1b shows similar biological activity to IFN-β-1a. The immunomodulatory activity appears to involve a dampening of the proinflammatory effects of IFN-γ, tumour necrosis factor (TNF)α, interleukin (IL)-12, lymphotoxin secretion; monocyte activation, preventing the disruption of the BBB and thereby reducing the entry of lymphocytes into the CNS; reducing antigen presentation to T-cells and up-regulating anti-inflammatory cytokines.

### 4. Corticosteroids (and ACTH)

These agents are currently used to treat acute relapses in relapsing-remitting MS and occasionally secondary progressive MS. Examples include methylprednisolone, prednisone, prednisolone, dexamethasone and adreno-corticotrophic hormone (ACTH or corticotropin), though use of the latter has been largely replaced by synthetic corticosteroids. The mechanism of action of corticosteroids in MS is thought to involve a reduction in BBB disruption, an inhibition of the Th1 immune response, a dampening of T-cell migration and response to antigens, suppression of adhesion molecule expression and protection of oligodendrocytes from cytokine-induced cell death.

### 5. Immunomodulators

### (a) Glatiramer Acetate (GA)

Glatiramer acetate (Copaxone®) is licensed for reduced frequency of relapses in relapsing-remitting multiple sclerosis. It is a random polymer (average molecular mass 6.4 kD) composed of four amino acids (alanine, glutamic acid, lysine, and tyrosine) in a particular molar ratio. The mechanism of action is unknown, but it is thought to promote a shift from pro-inflammatory Th1 to a regulatory Th2 type immune response that suppresses proinflammatory components. It may also act as a decoy, diverting an autoimmune response against myelin and acting as a sink for myelinolytic factors. It is administered by subcutaneous injection at a dose of 20 mg per day.

### (b) Chemotherapeutic Agents

Mitoxantrone is an anthracycline antineoplastic agent. It slows the progression of secondary progressive MS and extends the time between relapses in relapsing-remitting MS and progressive relapsing MS. Mitoxantrone is a type II topoisomerase inhibitor, disrupting DNA synthesis and DNA repair in both healthy cells and cancer cells. Other (currently unlicensed) chemotherapeutic agents include azathioprine, cyclophosphamide, cyclosporine, methotrexate and cladribine.

### (c) Antibodies

Natalizumab is a humanized monoclonal antibody against the cellular adhesion molecule α4-integrin. It is though to reduce the ability of inflammatory immune cells to attach to and pass through the cell layers lining the blood-brain barrier. Natalizumab is administered by intravenous infusion every 28 days.

Alemtuzumab (Millennium Pharmaceuticals, also known as Campath) is a humanized monoclonal antibody against CD52 which induces the secretion of TNF-α, IFN-γ and IL-6. It is currently undergoing clinical trials for the treatment of relapsing remitting MS. It finds particular application in combination with the auxiliary myelin repair agents discussed above and is particularly suitable for use in combination with the compounds of the invention.

### 6. Anti-Excitotoxics

Excitotoxicity is the pathological process by which nerve cells are damaged and killed by glutamate and similar substances attendant on overactivation of glutamate receptors (e.g. the NMDA receptor and AMPA receptors). Excitotoxins like NMDA and kainic acid which bind to these receptors, as well as pathologically high levels of glutamate, can cause excitotoxicity by allowing high levels of Ca2+ influx into cells which activates phospholipases, endonucleases and proteases such as calpain. These enzymes damage cell structures such as components of the cytoskeleton, membrane, and DNA. Excitotoxicity is involved in many myelinative diseases, including those associated with stroke, traumatic brain injury and neurodegenerative diseases of the central nervous system (CNS) such as Multiple sclerosis, Alzheimer's disease, Amyotrophic lateral sclerosis (ALS), Parkinson's disease, and Huntington's disease. NMDA antagonists such as eliprodil (a non-competitive, NR2B-selective NMDA antagonist) can delay neural cell death and may therefore act as neuroprotectants in the treatment of myelinative diseases (including MS).

Where appropriate, a reference to a particular adjunctive agent herein is intended to include ionic, salt, solvate, isomers, tautomers, N-oxides, ester, prodrugs, isotopes and protected forms thereof (preferably the salts or tautomers or isomers or N-oxides or solvates thereof, and more preferably, the salts or tautomers or N-oxides or solvates thereof).

It will be appreciated that two or more adjunctive agents may be used in combination with the compounds of the invention.

### Posology

The compounds of the present invention can be administered by oral or parenteral routes, including intravenous, intramuscular, intraperitoneal, subcutaneous, transdermal, airway (aerosol), rectal, vaginal and topical (including buccal and sublingual) administration.

The amount administered can vary widely according to the particular dosage unit employed, the period of treatment, the age and sex of the patient treated, the nature and extent of the disorder treated, and the particular compound selected.

Moreover, the compounds of the invention can be used in conjunction with other agents known to be useful in the treatment of diseases or disorders arising from protein folding abnormalities (as described infra) and in such embodiments the dose may be adjusted accordingly.

In general, the effective amount of the compound administered will generally range from about 0.01 mg/kg to 500 mg/kg daily. A unit dosage may contain from 0.05 to 500 mg of the compound, and can be taken one or more times per day. The compound can be administered with a pharmaceutical carrier using conventional dosage unit forms either orally, parenterally, or topically, as described below.

The preferred route of administration is oral administration. In general a suitable dose will be in the range of 0.01 to 500 mg per kilogram body weight of the recipient per day, preferably in the range of 0.1 to 50 mg per kilogram body weight per day and most preferably in the range 1 to 5 mg per kilogram body weight per day.

The desired dose is preferably presented as a single dose for daily administration. However, two, three, four, five or six or more sub-doses administered at appropriate intervals throughout the day may also be employed. These sub-doses may be administered in unit dosage forms, for example, containing 0.001 to 100 mg, preferably 0.01 to 10 mg, and most preferably 0.5 to 1.0 mg of active ingredient per unit dosage form.

### Formulation

The compound for use according to the invention may take any form. It may be synthetic, purified or isolated from natural sources.

When isolated from a natural source, the compound may be purified. In embodiments where the compound is formulated together with a pharmaceutically acceptable excipient, any suitable excipient may be used, including for example inert diluents, disintegrating agents, binding agents, lubricating agents, sweetening agents, flavouring agents, colouring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc.

The pharmaceutical compositions may take any suitable form, and include for example tablets, elixirs, capsules, solutions, suspensions, powders, granules and aerosols.

The pharmaceutical composition may take the form of a kit of parts, which kit may comprise the composition of the invention together with instructions for use and/or a plurality of different components in unit dosage form.

Tablets for oral use may include the compound for use according to the invention, mixed with pharmaceutically acceptable excipients, such as inert diluents, disintegrating agents, binding agents, lubricating agents, sweetening agents, flavouring agents, colouring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract. Capsules for oral use include hard gelatin capsules in which the compound for use according to the invention is mixed with a solid diluent, and soft gelatin capsules wherein the active ingredient is mixed with water or an oil such as peanut oil, liquid paraffin or olive oil.

Formulations for rectal administration may be presented as a suppository with a suitable base comprising for example cocoa butter or a salicylate. Formulations suitable for vaginal administration may be presented as pessaries, tampons, creams, gels, pastes, foams or spray formulations containing in addition to the active ingredient such carriers as are known in the art to be appropriate.

For intramuscular, intraperitoneal, subcutaneous and intravenous use, the compounds of the invention will generally be provided in sterile aqueous solutions or suspensions, buffered to an appropriate pH and isotonicity. Suitable aqueous vehicles include Ringer's solution and isotonic sodium chloride. Aqueous suspensions according to the invention may include suspending agents such as cellulose derivatives, sodium alginate, polyvinylpyrrolidone and gum tragacanth, and a wetting agent such as lecithin. Suitable preservatives for aqueous suspensions include ethyl and n-propyl p-hydroxybenzoate.

The compounds of the invention may also be presented as liposome formulations.

For oral administration the compound can be formulated into solid or liquid preparations such as capsules, pills, tablets, troches, lozenges, melts, powders, granules, solutions, suspensions, dispersions or emulsions (which solutions, suspensions dispersions or emulsions may be aqueous or non-aqueous). The solid unit dosage forms can be a capsule which can be of the ordinary hard- or soft-shelled gelatin type containing, for example, surfactants, lubricants, and inert fillers such as lactose, sucrose, calcium phosphate, and cornstarch.

In another embodiment, the compounds of the invention are tableted with conventional tablet bases such as lactose, sucrose, and cornstarch in combination with binders such as acacia, cornstarch, or gelatin, disintegrating agents intended to assist the break-up and dissolution of the tablet following administration such as potato starch, alginic acid, corn starch, and guar gum, lubricants intended to improve the flow of tablet granulations and to prevent the adhesion of tablet material to the surfaces of the tablet dies and punches, for example, talc, stearic acid, or magnesium, calcium, or zinc stearate, dyes, coloring agents, and flavoring agents intended to enhance the aesthetic qualities of the tablets and make them more acceptable to the patient.

Suitable excipients for use in oral liquid dosage forms include diluents such as water and alcohols, for example, ethanol, benzyl alcohol, and the polyethylene alcohols, either with or without the addition of a pharmaceutically acceptably surfactant, suspending agent or emulsifying agent.

The compounds of the invention may also be administered parenterally, that is, subcutaneously, intravenously, intramuscularly, or interperitoneally.

In such embodiments, the compound is provided as injectable doses in a physiologically acceptable diluent together with a pharmaceutical carrier (which can be a sterile liquid or mixture of liquids). Suitable liquids include water, saline, aqueous dextrose and related sugar solutions, an alcohol (such as ethanol, isopropanol, or hexadecyl alcohol), glycols (such as propylene glycol or polyethylene glycol), glycerol ketals (such as 2,2-dimethyl-1,3-dioxolane-4-methanol), ethers (such as poly(ethylene-glycol) 400), an oil, a fatty acid, a fatty acid ester or glyceride, or an acetylated fatty acid glyceride with or without the addition of a pharmaceutically acceptable surfactant (such as a soap or a detergent), suspending agent (such as pectin, carhomers, methylcellulose, hydroxypropylmethylcellulose, or carboxymethylcellulose), or emulsifying agent and other pharmaceutically adjuvants. Suitable oils which can be used in the parenteral formulations of this invention are those of petroleum, animal, vegetable, or synthetic origin, for example, peanut oil, soybean oil, sesame oil, cottonseed oil, corn oil, olive oil, petrolatum, and mineral oil. Suitable fatty acids include oleic acid, stearic acid, and isostearic acid. Suitable fatty acid esters are, for example, ethyl oleate and isopropyl myristate.

Suitable soaps include fatty alkali metal, ammonium, and triethanolamine salts and suitable detergents include cationic detergents, for example, dimethyl dialkyl ammonium halides, alkyl pyridinium halides, and alkylamines acetates; anionic detergents, for example, alkyl, aryl, and olefin sulphonates, alkyl, olefin, ether, and monoglyceride sulphates, and sulphosuccinates; nonionic detergents, for example, fatty amine oxides, fatty acid alkanolamides, and polyoxyethylenepolypropylene copolymers; and amphoteric detergents, for example, alkyl-beta-aminopropionates, and 2-alkylimidazoline quarternary ammonium salts, as well as mixtures.

The parenteral compositions of this invention will typically contain from about 0.5 to about 25% by weight of the compound for use according to the invention in solution. Preservatives and buffers may also be used. In order to minimize or eliminate irritation at the site of injection, such compositions may contain a non-ionic surfactant having a hydrophile-lipophile balance (HLB) of from about 12 to about 17. The quantity of surfactant in such formulations ranges from about 5 to about 15% by weight. The surfactant can be a single component having the above HLB or can be a mixture of two or more components having the desired HLB. Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The compound for use according to the invention may also be administered topically, and when done so the carrier may suitably comprise a solution, ointment or gel base. The base, for example, may comprise one or more of the following: petrolatum, lanolin, polyethylene glycols, bee wax, mineral oil, diluents such as water and alcohol, and emulsifiers and stabilizers. Topical formulations may contain a concentration of the compound from about 0.1 to about 10% w/v (weight per unit volume).

When used adjunctively, the compound for use according to the invention may be formulated for use with one or more other drug(s). In particular, the compounds may be used in combination with lysosomal enzymes adjunctive to enzyme replacement therapy. Thus, adjunctive use may be reflected in a specific unit dosage designed to be compatible (or to synergize) with the other drug(s), or in formulations in which the compound is admixed with one or more enzymes. Adjunctive uses may also be reflected in the composition of the pharmaceutical kits of the invention, in which the compounds of the invention is co-packaged (e.g. as part of an array of unit doses) with the enzymes. Adjunctive use may also be reflected in information and/or instructions relating to the co-administration of the compound and/or enzyme.

### EXEMPLIFICATION

The invention will now be described with reference to specific Examples. These are merely exemplary and for illustrative purposes only: they are not intended to be limiting in any way to the scope of the monopoly claimed or to the invention described. These examples constitute the best mode currently contemplated for practicing the invention.

### Example 1

### Assay for Screening Inhibitor Library Against Human OGA

Expressed and purified human OGA (hOGA) was screened against a library of compounds (compounds stored in 100% DMSO at a concentration of 10 mM) according to a procedure modified from that detailed in Biochem. J. 2009, 420, 221-227 and using the reaction reagents set out in the Table (below). The modification to the published protocol was that an emission wavelength of 450 nM was used rather than 460 nM. Assays were run at singlepoint in triplicate. Inhibitors for further evaluation were selected based on an average observed inhibition greater than 50%.

### Example 2

### Assay for Determination of IC₅₀ Values for Inhibition of Human OGA

Inhibitors identified from the screen described in Example 1 were evaluated for IC₅₀ using a procedure adapted from Biochem. J. 2009, 420, 221-227. IC₅₀ determination was carried out using a range of inhibitor concentrations from 300 μM to 0.003 μM or 100 μM to 0.001 μM. IC₅₀ values were calculated using XLFit 4. When tested in the assay described above some of the compounds described herein exhibit IC₅₀ values for the inhibition of human OGA in the range 0.001-300 μM.

### Example 3

### Assay for Determination of IC₅₀ Values for Inhibition of Human β-Hexosaminidase

Inhibitors identified from the screen described in Example 1 were evaluated for IC₅₀ against human β-hexosaminidase [human placenta, Sigma-Aldrich] using a range of inhibitor concentrations from 300 μM to 0.003 μM or 100 μM to 0.001 μM and the following reaction conditions:

Reactions were carried out at room temperature for thirty minutes and then quenched through the addition of 150 μl 0.5M Na₂CO₃. Fluorescence readings were taken at excitation wavelength 360 nm and emission wavelength 450 nm. IC₅₀ values were calculated using XLFit 4. The selectivity ratio for inhibition of human O-GlcNAcase over human β-hexosaminidase is defined here as:

IC₅₀(human β-hexosaminidase)/IC₅₀(Human OGA)

Inhibition data for selected compounds [% human OGA inhibition at 100 μM inhibitor concentrtaion; IC₅₀ against human OGA; IC₅₀ against HexA/B; Selectivity] is shown in the Table below:

| | | | HEXA/B | |
|---|---|---|---|---|
| | OGA Inhibition | | Inhibition | |
| Compound ID | Inhibition (%) | IC₅₀ (μM) | IC₅₀ (μM) | Selectivity |
| A001 | 89.6 | 57.983 | >300 | >5 |
| A002 | 52.8 | 74.955 | >300 | >4 |
| A003 | 82.8 | 74.644 | >300 | >4 |
| A004 | ND | 0.537 | >300 | >555 |
| A005 | ND | 0.144 | >300 | >2142 |
| A006 | ND | 1.248 | >300 | >234 |
| A007 | ND | 0.102 | >300 | >3000 |
| A008 | ND | 0.012 | >300 | >25000 |
| A009 | ND | 0.036 | 197 | 5472 |
| A010 | ND | 0.009 | 33 | 3666 |
| A011 | ND | 0.033 | >300 | >9174 |
| A012 | ND | 0.049 | >300 | >6085 |
| A013 | ND | 130 | >300 | >2 |
| A014 | ND | 3.66 | >300 | >81 |
| A015 | ND | <0.003 | 11 | >3666 |
| A016 | ND | <0.003 | 54 | >18000 |
| A017 | ND | 0.056 | 196 | 3500 |
| A018 | ND | 0.014 | 69 | 5000 |
| A019 | ND | 0.069 | >300 | >4366 |
| A020 | ND | 0.023 | >300 | >13215 |
| A021 | ND | 0.008 | >300 | >37037 |
| A022 | ND | 0.011 | >300 | >28571 |
| A023 | ND | 0.048 | >300 | >6211 |
| A024 | ND | 143 | 44 | 0.31 |
| A025 | ND | 129 | >300 | >2.3 |
| A026 | ND | 4.39 | >300 | >68.34 |
| A027 | ND | 11.56 | >300 | >25.95 |
| A028 | ND | 215 | 300 | >1.39 |
| A029 | ND | 125 | >300 | >2.38 |
| A030 | ND | 94.93 | >300 | >3.16 |
| A031 | ND | 75.5 | >300 | >3.97 |
| A032 | ND | 6.07 | >300 | >49.4 |
| A033 | ND | 1.68 | >300 | >178.57 |
| A034 | ND | 68 | >300 | >4.41 |
| A035 | ND | 3.9 | >300 | >76.92 |
| A036 | ND | 136.15 | 73 | 0.54 |
| A037 | ND | 0.228 | >300 | >1315 |
| A038 | ND | 1.3 | >300 | >230 |
| A039 | ND | 1.59 | >300 | >188.68 |
| A040 | ND | 0.045 | 151 | 3355 |
| A041 | ND | 0.014 | 114 | 8142 |
| A042 | ND | 2.8 | 2.5 | 0.89 |
| A043 | ND | 10.2 | 14 | 1.37 |
| A044 | ND | 0.0279 | 0.13 | 4.66 |
| A045 | ND | 0.586 | 2.76 | 4.71 |
| A046 | ND | 0.0237 | 0.0099 | 0.42 |
| A047 | ND | 0.926 | 0.4051 | 0.44 |
| A048 | ND | 23.8 | >300 | >12.6 |
| A049 | ND | 1.06 | >300 | >283 |
| A050 | ND | 0.412 | >300 | >728 |
| A051 | ND | 28.76 | >300 | >10.43 |
| A052 | ND | 0.113 | >300 | >2654 |
| A053 | ND | 0.0629 | >300 | >4769 |
| A054 | ND | 6.98 | >300 | >42.98 |
| A055 | ND | 0.1186 | >300 | >2529 |
| A056 | ND | 0.58 | 0.0107 | 0.02 |
| A057 | ND | 39.2 | 4.256 | 0.11 |
| A058 | ND | 0.228 | 0.031 | 0.14 |
| A059 | ND | 0.007 | 0.0299 | 4.27 |
| A060 | ND | 0.011 | >300 | >27272 |
| A061 | ND | 0.0693 | >300 | >4329 |
| A062 | ND | 0.512 | >300 | >585 |
| A063 | ND | 0.036 | >300 | >8333 |
| A064 | ND | <0.003 | 10.1 | >3366 |
| A065 | ND | 0.007 | 58.4 | 8342 |
| A066 | ND | <0.003 | 10.9 | >3633 |
| A067 | ND | <0.003 | 15 | >5000 |
| A068 | ND | 0.0037 | 242 | >65405 |
| A069 | ND | 224 | >300 | >1.34 |
| A070 | ND | 0.0075 | 166 | 22133 |
| A071 | ND | 0.007 | >300 | >42857 |
| A072 | ND | 0.002 | >300 | >150000 |

### Example 4

### Assay for Determination of Kᵢ Values for Inhibition of Human OGA

Enzymatic reactions were carried out in McIlvaine buffer (0.2M Na₂PO₄, 0.1M citric acid; pH 7.4) using 2 nM human OGA and 4MU-GlcNAc as substrate in the presence of inhibitor. For the Ki of AXX inhibitor concentrations were 0, 20, 40, 80, 160 and 320 nM [for the Ki determination of other compounds either the same concentrations or similar concentrations were used] and 4MU-GlcNAc concentrations were 50, 100, 200, 400, 800, 1600, and 3200 μM. Reactions were left for one hour. Time dependent assay of OGA revealed that the enzyme was stable over the reaction time and in the reaction conditions. Modes of inhibition were determined by Lineweaver-Burke analysis and Ki values were determined using GraphPad 5 Prism non-linear regression analysis.

When tested in the assay described above some of the compounds described herein exhibit Kᵢ values for the inhibition of human OGA in the range 0.003-0.2 μM, as shown in the Table, below:

| Compound ID | Kᵢ [human OGA] μM |
|---|---|
| A008 | 0.055 |
| A012 | 0.070 |
| A016 | 0.011 |
| A021 | 0.041 |
| A022 | 0.072 |
| A066 | 0.003 |
| A060 | 0.167 |
| A068 | 0.064 |

### Example 5

### Western Blot Analyses

PC-12 cells or SH-SY5Y cells were dosed with the test compound or vehicle control at 37° C. for 4 hours. Once the incubation was completed the media was removed and cells were lysed. Extracted protein was then used for Western blot analysis using standard molecular biology techniques. The resulting membrane was probed with antibodies specific for tau phosphorylation sites (pSer396, pSer422, pThr231, pSer262) or with antibodies specific for O-GlcNAcylation sites (RL-2 or CTD110.6). The primary antibodies were visualised using appropriate secondary conjugated with the fluorophores Alexa 532 or Alexa 488. Actin was visualised using a FITC conjugated primary antibody. Relative quantification was measured in relation to the actin signal using Image lab software (BRL). Marked increases in O-GlcNAcylated proteins within the cells were observed as compared to controls when cells were dosed with test compound.

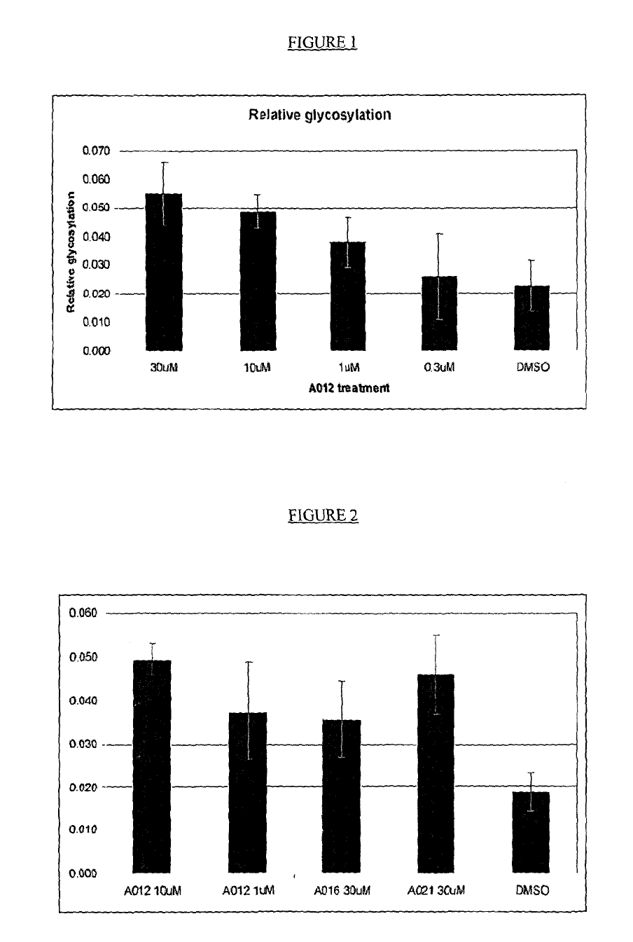

The results are shown in FIGS. 1 and 2, where FIG. 1 shows quantification of western blot analysis of proteins from SH-SY5Y cells cultured for 4 hours in the presence of varying concentrations of compound A012. Incubation of SH-SY5Y cells with compound A012 caused an increase in cellular levels of O-GlcNAc modified proteins relative to vehicle.

FIG. 2 shows quantification of western blot analysis of proteins from SH-SY5Y cells cultured for 4 hours in the presence of compounds A012, A016 and A021. Incubation of SH-SY5Y cells with compounds A012, A016, A021 caused an increase in cellular levels of O-GlcNAc modified proteins relative to vehicle.

EC₅₀ values were calculated using Graph Pad 5 Prism and results are shown in the Table below:

| Compound ID | Ec₅₀ μM |
|---|---|
| A007 | 4 |
| A010 | 1.54 |
| A012 | 0.656 |
| A015 | 0.08 |
| A016 | 0.255 |
| A018 | 0.931 |
| A019 | 1.9 |
| A021 | 0.604 |
| A022 | 1.23 |
| A050 | 2.72 |
| A052 | 1.07 |
| A053 | 1.409 |
| A055 | 3.72 |
| A060 | 0.263 |
| A063 | 1.01 |
| A064 | 0.127 |
| A066 | 0.020 |

### Example 6

### Synthesis of Compounds of General Formula A

Compounds of the invention having general formula A are prepared according to the sequence detailed in General Scheme 1 and Scheme 1 and Scheme 2:

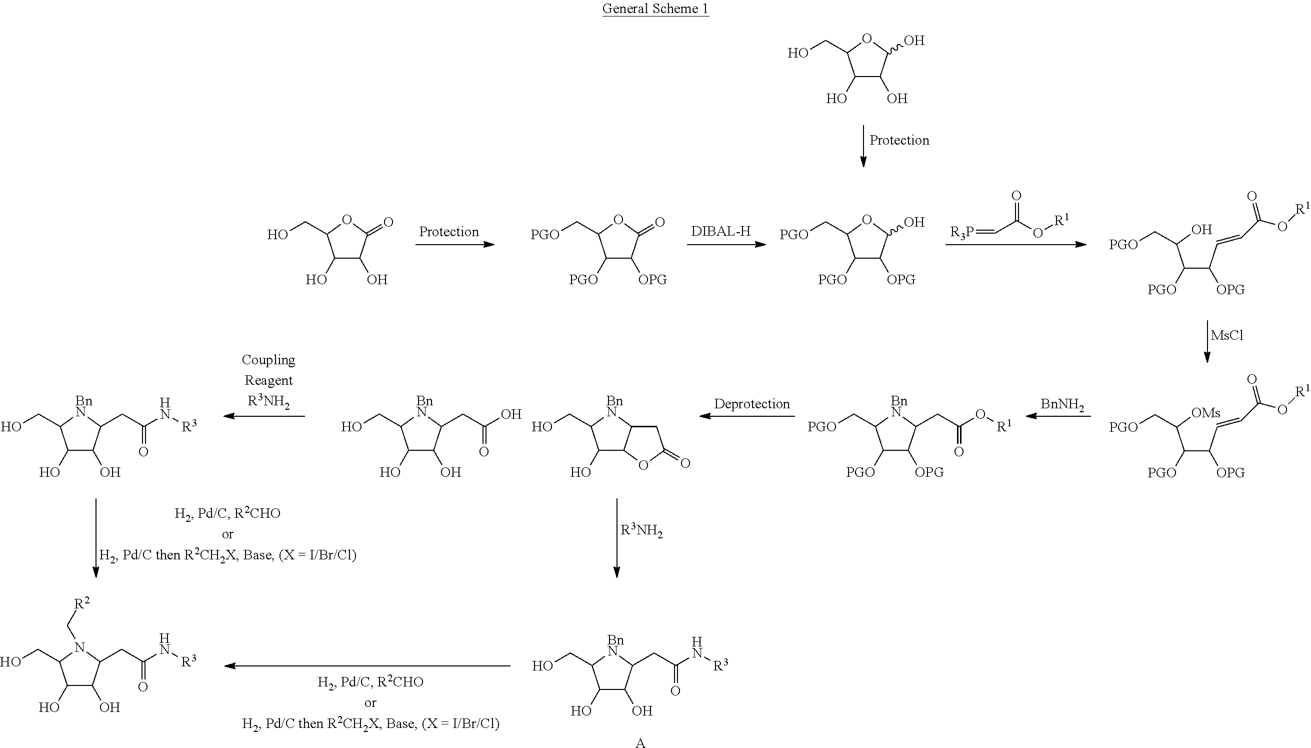

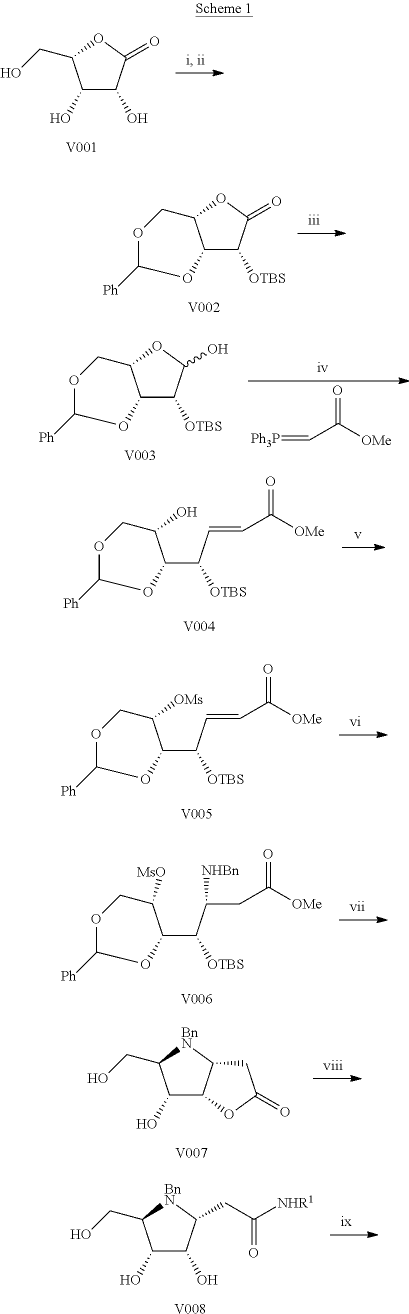

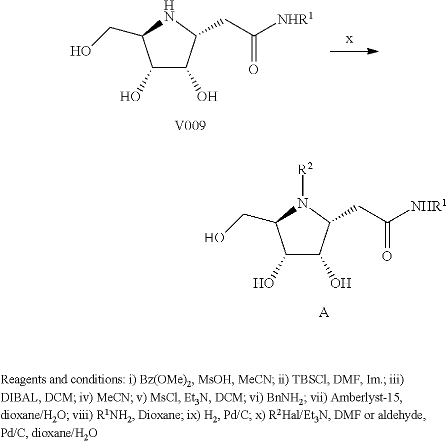

Steps i, ii: To a solution of V001 (0.0885 mol, 1 eq) and Bz(OMe)₂ (0.0974 mmol, 1.1 eq) in MeCN (300 mL) was added MsOH (catalytic) at rt. After 3 h the reaction mixture was filtered and the solids washed with PE. The solids were triturated with PE/EtOAc to furnish the desired product (75%). The product (0.032 mol, 1 eq) was dissolved in DMF (100 mL) and TBSCl (0.039 mol, 1.2 eq) and imidazole (0.078 mol, 2.4 eq) were added. The reaction mixture was stirred at rt for 16 h, after which it was diluted with EtOAc. The organic solution was washed twice with water and the aqueous phase back extracted with EtOAc. The combined organic layers were washed with brine, dried over MgSO₄ and concentrated in vacuo. The crude was purified by column chromatography (PE/EtOAc, 1/10 to 1/1) to yield the desired product (100%).

Step iii: To a solution of V002 (0.032 mol, 1 eq) in CH₂Cl₂, at 0° C. under argon, was added DIBAL-H (0.038 mol, 1.2 eq). After 1 h at 0° C., 1 M H₂SO₄ was added and the product extracted with CH₂Cl₂ (×3), dried over MgSO₄ and concentrated in vacuo to yield the desired product (100%).

Step iv: Methyl (triphenylphosphoranylidene) acetate (35 mmol, 1.1 eq) was added to a solution of V003 (32 mmol, 1 eq) in MeCN (100 mL) and the reaction mixture heated at 60° C. for 2 h. Concentration in vacuo followed by column chromatography (PE/EtOAc, 1/0 to 7/3) yielded the desired product (25%).

Step v: To a stirred solution of V004 (7.8 mmol, 1 eq) in pyridine (40 mL) at rt was added MsCl (15.6 mmol, 2 eq) and DMAP (catalytic). After 2 h the reaction mixture was quenched with MeOH and concentrated in vacuo The crude was taken up in EtOAc, washed with NaHCO₃ (×2), the aqueous back extracted with EtOAc, the combined organic layer washed with brine, dried over MgSO₄ and the solvent removed in vacuo. The crude was purified by column chromatography (PE/EtOAc, 1/0 to 7/3) to yield the desired product (92%).

Step vi: A solution of V005 (7.2 mmol, 1 eq) in benzylamine was heated to 60° C. overnight. The reaction mixture was concentrated in vacuo by co-evaporation with toluene (×3). The crude was purified by column chromatography (PE/EtOAc, 1/0 to 7/3) to yield the desired product (55%).

Step vii: To a solution of V006 (3.66 mmol, 1 eq) in dioxane/water (20 mL, 1/1) was added Amberlyst-15 and the reaction mixture heated to 95° C. overnight. Once cool, 2 M NH₄OH/MeOH was added and the suspension filtered and concentrated in vacuo using toluene as a co-solvent (×3). The crude was purified by column chromatography (PE/EtOAc, 1/0 to 0/1) to yield the desired product (43%).

Step viii: A solution of V007 (1.43 mmol, 1 eq) and R¹NH₂ (4.28 mmol, 3 eq) in dioxane (20 mmol) was heated to 60° C. overnight. The reaction mixture was concentrated in vacuo and purified by column chromatography (PE/EtOAc, 1/0 to 85/15) to yield the desired product.

Step ix: A suspension of V008 (1.28 mmol, 1 eq) and Pd black (catalytic) in MeOH (10 mL) was stirred under a H₂ atmosphere for 16 h. The reaction mixture was filtered through celite and the filtrate concentrated in vacuo to yield the desired product.

Step x: Procedure A: To a solution of V009 (0.12 mmol, 1 eq) in DMF (2 mL) was added R²Hal (0.018 mmol, 1.5 eq) and Et₃N (0.36 mmol, 3 eq). The reaction mixture was heated to 60° C. overnight, before concentrating in vacuo. The crude was purified using an SCX-2 cartridge (1% NH₃/MeOH) to yield the desired product.

Procedure B: To a solution of V009 (0.12 mmol, 1 eq) in dioxane (2 mL) was added the requisite aldehyde (0.018 mmol, 1.5 eq) and Pd/C (catalytic). The reaction mixture was stirred under a hydrogen atmosphere at room temperature overnight, before filtration through Celite and concentrating in vacuo. The crude was purified using an SCX-2 cartridge (Biotage) eluting with 1% NH₃/MeOH to yield the desired product.

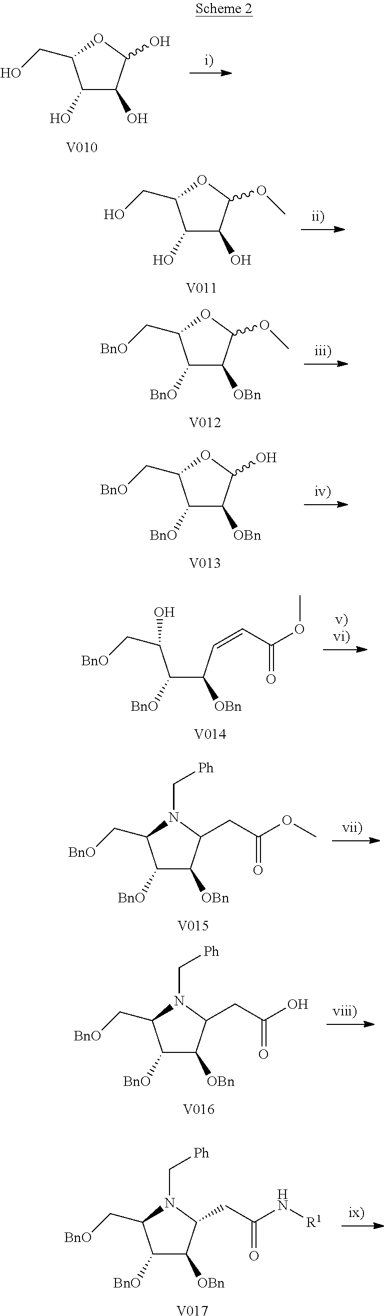

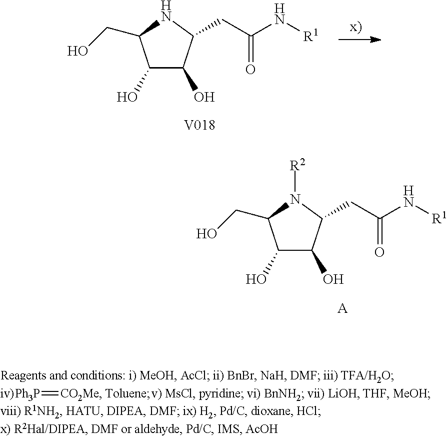

Step i: To a suspension of V010 (10.0 g, 66.61 mmol) in methanol (100 mL) was added acetyl chloride (950 uL, 13.32 mmol) and stirred at room temperature for 5 hours. Silver carbonate (10 g, 33 mmol) was added and stirred for 1 hour then filtered. Liquors were absorbed onto silica and purified by flash chromatography eluting with 5% increasing to 20% methanol in ethyl acetate. Title product V011 isolated as a colourless oil 7.91 g, 48 mmol (73% yield).

Step ii: To a solution of V011 (7.91 g, 48 mmol) in anhydrous DMF (250 mL), under argon at 0° C. was added solid sodium hydride (6.75 g, 169 mmol) in portions over 25 minutes then stirred for 30 minutes. Benzyl bromide (22.8 mL, 192 mmol) was added slowly over 30 minutes then warmed to room temperature and stirred for 18 hours. The majority of DMF was removed in-vacuo and the residue dissolved in chloroform. Organics washed with water then brine and dried over MgSO4. Purified by flash chromatography eluting with neat petroleum 40-60° C. increasing to 25% ethyl acetate in petroleum. V012 (as a mixture of anomers) was isolated as a pale yellow oil 21.3 g, 48 mmol (quantitative yield).

Step iii: A solution of V012 (10 g, 23 mmol) in (2:1) water:TFA (75 mL total) was stirred vigorously at 60° C. for 5 hours. The reaction mixture was cooled and transferred to a large beaker before carefully quenching with solid NaHCO₃ until basic (pH˜9). Aqueous extracted with ethyl acetate (3×100 mL), combined organics were washed with brine and dried over MgSO4 and filtered. Liquors were absorbed onto silica and purified by flash chromatography eluting with 10% increasing to 33% ethyl acetate in petroleum V013 isolated as a colourless oil 4.94 g, 11.8 mmol (51% yield).

Step iv: Methyl (triphenylphosphoranylidene) acetate (10.94 mmol, 1.2 eq) was added to a solution of V013 (9.12 mmol, 1 eq) in toluene (150 mL) and the reaction mixture heated at 60° C. for 2 h. Reaction was cooled, concentrated and absorbed onto silica and purified by column chromatography (PE/EtOAc, 80/20 to 66/33) yielding the desired product V014 (76%).

Steps v and vi: To a stirred solution of V014 (6.93 mmol, 1 eq) in pyridine (40 mL) at rt was added MsCl (10.4 mmol, 1.5 eq) and the reaction mixture was stirred at 50° C. overnight. The reaction mixture was concentrated under reduced pressure with the resulting residue taken up in benzylamine (20 ml) and heated at 50° C. overnight with stirring. The mixture was concentrated under reduced pressure and the residue partitioned between ethyl acetate and water with the organic layer washed with brine, dried over MgSO₄, filtered and absorbed onto silica. Crude material was purified by silica gel chromatography PE/EtOAc, 1/0 to 85/15) providing V015 as a colourless oil (37%).

Step vii: V015 (2.4 mmol) was dissolved in THF/MeOH/LiOH-1M (25/5/25) and stirred vigourously overnight. Volatiles were removed under reduced pressure and the remaining solution was acidified (HCl-5M) and extracted with ethyl acetate. Organics were washed with brine, dried over MgSO₄ filtered and concentrated affording V016 as a brown oil (quantitative)

Step viii: To a solution of V016 (2.41 mmol) and HATU (3.37 mmol) in DMF (20 ml) and DIPEA (7.23 mmol) was added R¹NH₂(9.64 mmol) and the reaction was stirred overnight at room temperature. The reaction mixture was concentrated and the resulting residue was dissolved in chloroform and washed with water, NaHCO₃, brine, dried (MgSO₄) filtered and concentrated under reduced pressure. Material was purified by column chromatography (PE/EtOAc, 1/1) affording V017 as colourless oil (44%).

Step ix: A suspension of V017 (0.796 mmol, 1 eq) and Pd black (catalytic) in dioxane (15 mL) and HCl-1M (1.5 ml) was stirred under a H₂ atmosphere for 16 h. The reaction mixture was filtered through celite and the filtrate concentrated in vacuo to yield the desired product V018 that was further purified by SCX-2 cartridge eluting with NH₃ in MeOH (95% yield).

Step x: Procedure A: To a solution of V017 (1 eq) in DMF (3 mL) was added R²Hal (1.5 eq) and DIPEA (2 eq). The reaction mixture was heated to 70° C. overnight, before concentrating in vacuo. The crude was purified using an SCX-2 cartridge (1% NH₃/MeOH) to yield the desired product.

Procedure B: To a solution of V017 (1 eq) in IMS (2 mL) and AcOH (2 drops) was added the requisite aldehyde (1.2 eq) and Pd black (catalytic). The reaction mixture was stirred under a hydrogen atmosphere at room temperature overnight, before filtration through Celite and concentrating in vacuo. The crude was purified using either an SCX-2 cartridge (Biotage) eluting with 1% NH₃/MeOH to yield the desired product or by column chromatography.

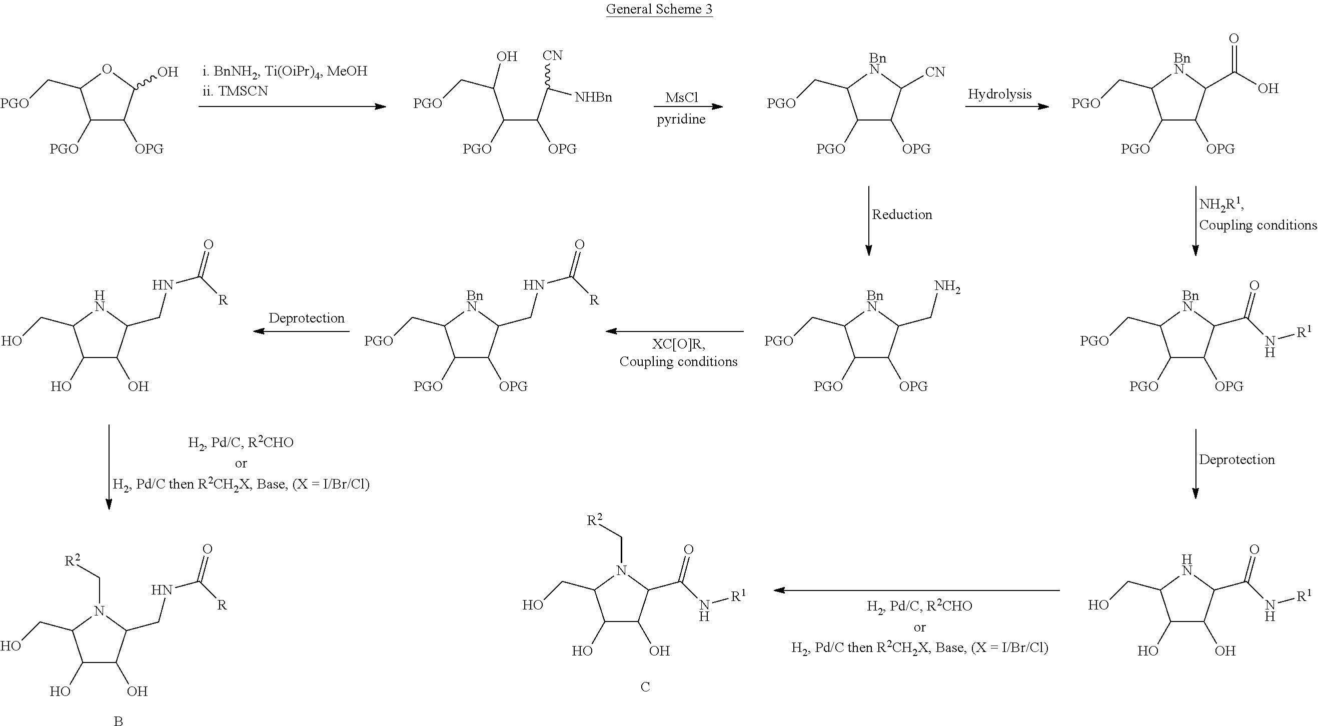

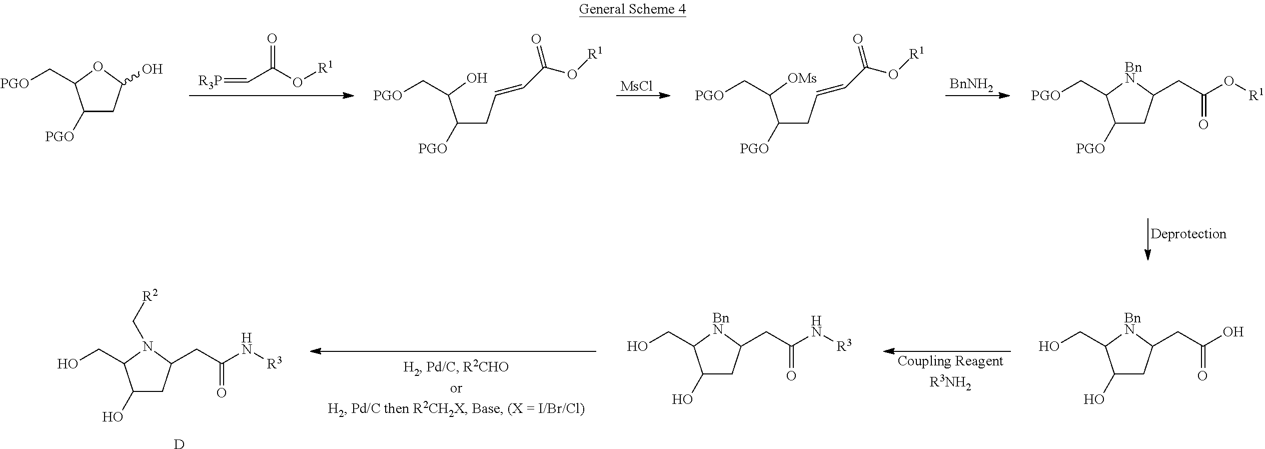

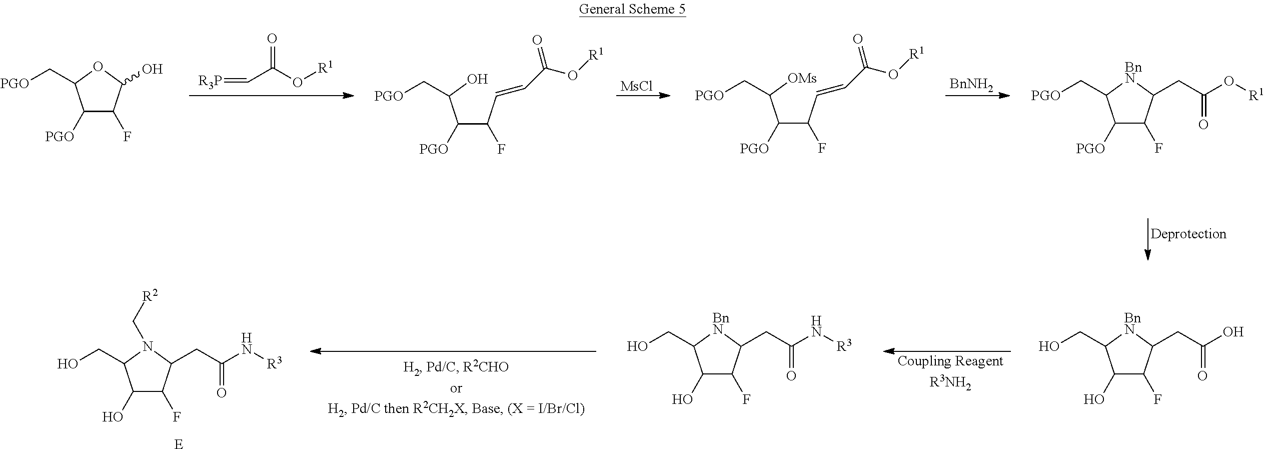

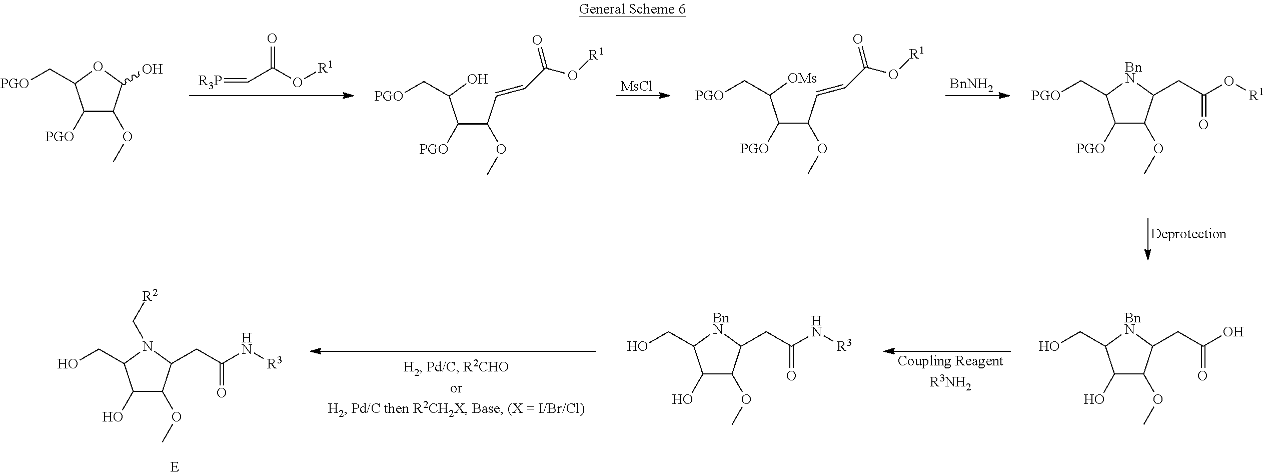

### Example 7

### Synthesis of Reverse Amides

Compounds of the invention having general formula B, including for example compounds A034, A036 and A039, can be prepared as described below:

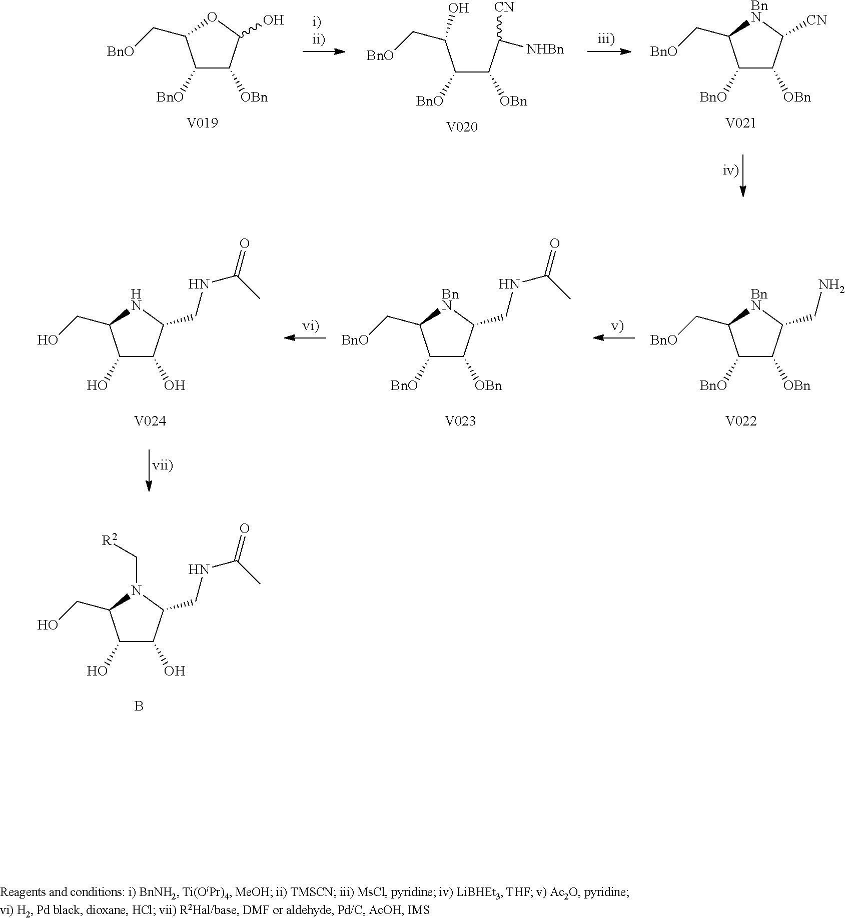

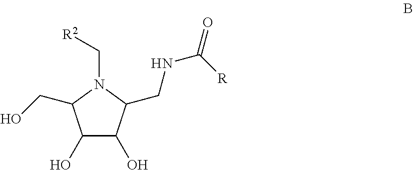

Steps i and ii: Bezylamine (1.45 mL, 13.3 mmol) and Ti(O/Pr)₄ (4.05 mL, 13.3 mmol) were added to a stirred solution of V019 (2.8 g, 6.67 mmol) in methanol (25 mL). The resulting mixture was allowed to stir at room temperature overnight. TMSCN (1.66 mL, 13.3 mmol) was then added in one portion and the resulting mixture allowed to stir at room temperature for a further 5 hours. Water (˜10 mL) and ethyl acetate (50 mL) were then added, and the resulting precipitate removed by filtration through celite with methanol and ethyl actetate. The filtrate was then concentrated in vacuo and purified using flash chromatography (10%-30% EtOAc in pet. ether 40-60°) to afford nitrile V020 as an orange oil (3.03 g, 85%).

Step iii: Mesyl chloride (205 μL, 2.64 mmol) was added to a solution of nitrile V020 (470 mg, 0.88 mmol) in pyridine (6.5 mL) and the resulting solution allowed to stir at 80° C. for 1.5 hours. A colour change from orange to brown was observed. The reaction mixture was allowed to cool to room temperature, poured into NaHCO_{3 (sat)} (40 mL) and extracted with EtOAc (40 mL). The organic extract was then washed with NaHCO_{3 (sat)} (40 mL), water (2×40 mL), dried (MgSO₄) and concentrated in vacuo. Purification by flash chromatography (0-20% EtOAc in pet. ether 40-60°) afforded pyrrolidine V021 as a yellow oil (374 mg, 82%).

Step iv: Super hydride (LiBHEt₃) (7 mL, 7 mmol) was added dropwise to a solution of nitrile V021 (365 mg, 0.7 mmol) in THF (12 mL) at 0° C. and the resulting mixture allowed to stir at this temperature for 2.5 hours. The reaction mixture was then poured into water (40 mL) and extracted with EtOAc (40 mL). The organic extract was then washed with 1N HCl (40 mL) and NaHCO_{3 (sat)} (40 mL), dried (MgSO₄), concentrated in vacuo and purified on SCX to afford amine V022 as a yellow oil (282 mg, 81%)

Step v: Acetic anhydride (0.5 mL, excess) was added to a solution of amine V022 (282 mg, 0.54 mmol) in pyridine (5 mL) and the resulting mixture allowed to stir at room temperature for 4 hours. The reaction was then quenched by addition of MeOH (10 mL) and concentrated in vacuo. Purification by flash chromatography (30% EtOAc in pet. ether) 40-60° afforded reverse amide V023 as a colourless oil (200 mg, 66%).

Step vi: A solution of reverse amide V023 (200 mg, 0.35 mmol), palladium black (5-10 mg, cat.) and 1N HCl (1.06 mL, 1.06 mmol) in dioxane (5 mL) was allowed to stir under an atmosphere of hydrogen overnight. The reaction mixture was then filtered through celite with MeOH and the filtrate concentrated in vacuo. Purification by SCX (gradient of 0.7 N NH₃ in MeOH to 7 N NH₃ in MeOH) afforded V024 as a pale brown oil (70 mg, 98%).

Step vii: Procedure A: To a solution of V024 (1 eq) in DMF (3 mL) was added R²Hal (1.5 eq) and base (2 eq). The reaction mixture was heated to 70° C. overnight, before concentrating in vacuo. The crude was purified using an SCX-2 cartridge (1% NH₃/MeOH) to yield the desired product.

Procedure B: To a solution of V024 (1 eq) in IMS (2 mL) and AcOH (2 drops) was added the requisite aldehyde (1.2 eq) and Pd/C (catalytic). The reaction mixture was stirred under a hydrogen atmosphere at room temperature overnight, before filtration through Celite and concentrating in vacuo. The crude was purified using either an SCX-2 cartridge (Biotage) eluting with 1% NH₃/MeOH to yield the desired product or by column chromatography.

### EQUIVALENTS

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

1. A compound of Formula (1) 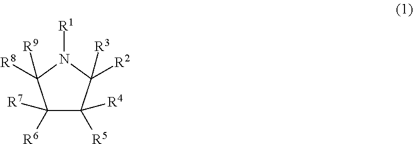
in which
R¹ represents H; C1-15 alkyl, C1-15 alkenyl or C1-15 alkynyl, optionally substituted with one or more R³; oxygen or an oxygen containing group such that the compound is an N-oxide; C(O)OR¹⁰; C(O)NR¹⁰R¹¹; SO₂NR¹⁰; SO₂R¹⁰; OH; OR¹⁰; or formyl
R² represents C(O)NR¹⁰R¹¹; CR¹⁰R¹¹C(O)NR¹⁰R¹¹; C(S)NR¹⁰R¹¹; CR¹⁰R¹¹C(S)NR¹⁰R¹¹; CF₂NR¹⁰R¹¹; CR¹⁰R¹¹CF₂NR¹⁰R¹¹; C(R¹⁰)═CR¹⁰R¹¹; CR¹⁰R¹¹C(R¹⁰)═CR¹⁰R¹¹; SO₂NR¹⁰R¹¹; CR¹⁰R¹¹SO₂NR¹⁰R¹¹; NR¹⁰C(O)NR¹⁰R¹¹; CR¹⁰R¹¹NR¹⁰C(O)NR¹⁰R¹¹; NR¹⁰C(═NR¹⁰)NR¹⁰R¹¹; CR¹⁰R¹¹NR¹⁰C(═NR¹⁰)NR¹⁰R¹¹; CR¹⁰(CF₃)NR¹⁰R¹¹; NR¹⁰C(O)R¹⁰; NR¹⁰C(S)R¹⁰; CR¹⁰R¹¹NR¹⁰C(O)R¹⁰; CR¹⁰R¹¹NR¹⁰C(S)R¹⁰; NR¹⁰SO₂R¹⁰; CR¹⁰R¹¹NR¹⁰SO₂R¹⁰; NR¹⁰C(CF₃)R¹⁰; CR¹⁰R¹¹NR¹⁰C(CF₃)R¹⁰; NR¹⁰CF₂R¹⁰; CR¹⁰R¹¹NR¹⁰CF₂R¹⁰;
each substituent R³, R⁴, R⁵, R⁶, R⁷, R⁸, R⁹ is selected independently from each other from a group consisting of H; OH; OR¹⁰; ═O; NH₂; N₃; SH; SOₓR¹⁰; halo; CN; NO₂; NR¹⁰R¹¹; (NR¹⁰)NR¹⁰R¹¹; NH(NR¹⁰_{)NR}¹⁰R¹¹; CO₂R¹⁰; OC(O)R¹⁰; P(O)(OR¹⁰)₂; C1-15 alkyl or alkenyl optionally substituted with one or more OH, OR¹⁰, ═O, NH₂, N₃, SH, SOₓR¹⁰, halo, CN, NO₂, NR¹⁰R¹¹, (NR¹⁰)NR¹⁰R¹¹, NH(NR¹⁰)NR¹⁰R¹¹, CO₂R¹⁰, OC(O)R¹⁰, P(O)(OR¹⁰)₂, aryl or carbocyclyl groups; carbocyclyl or aryl, either of which is optionally substituted with one or more OH, OR¹⁰, ═O, NH₂, N₃, SH, SOₓR¹⁰, halo, CN, NO₂, NR¹⁰R¹¹, (NR¹⁰)NR¹⁰R¹¹, NH(NR¹⁰)NR¹⁰R¹¹, CO₂R¹⁰, OC(O)R¹⁰, P(O)(OR¹⁰)₂, C1-9 alkyl optionally substituted with one or more OH, OR¹⁰, ═O, NH₂, N₃, halo, CN, NO₂, NR¹⁰R¹¹, CO₂R¹⁰, aryl or carbocyclyl groups; O-glycosyl; C-glycosyl; O-sulfate; O-phosphate or a group which together with the endocyclic carbon forms a spiro ring
R¹⁰ represents H; C1-6 alkyl, optionally substituted with one or more OH, halo; aryl or C1-4 alkyl optionally substituted with aryl; and
R¹¹ represents H; C1-6 alkyl, optionally substituted with one or more OH
R¹⁰ and R¹¹ may optionally form a 3 to 8 membered ring, containing one or more O, SOₓ or NR¹⁰ groups
x represents an integer from 0 to 2
or a pharmaceutically acceptable salt, derivative, solvate, isomer, tautomer, N-oxide, ester, prodrug, isotope or protected form thereof
with the provisos that: (a) two OH groups may not be attached to the same endocyclic carbon atom; (b) where there is only one R³, R⁴, R⁵, R⁶, R⁷, R⁸, R⁹ substituent it contains an oxygen atom directly bonded to an endocyclic carbon atom; (c) any two from R³, R⁴, R⁵, R⁶, R⁷, R⁸, R⁹ substituents may together form an optionally heterocyclic ring (for example a carbocycle, cyclic ether or acetal); and (d) the compound is not selected from the following compounds:

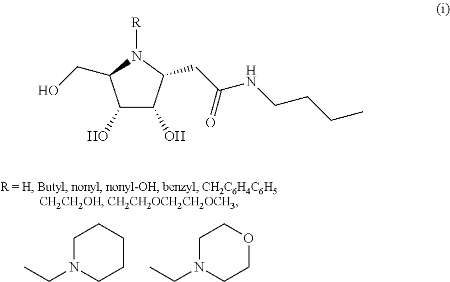

2. The compound of claim 1 wherein R¹ represents C1-15 alkyl, C1-15 alkenyl or C1-15 alkynyl substituted with one or more R³.

3. The compound of claim 2 wherein R³ is selected from carbocyclyl or aryl, either of which is optionally substituted with one or more OH, OR¹⁰, ═O, NH₂, N₃, SH, SOₓR¹⁰, halo, CN, NO₂, NR¹¹R¹¹, (NR¹⁰)NR¹⁰R¹¹, NH(NR¹⁰)NR¹⁰R¹¹, CO₂R¹⁰, OC(O)R¹⁰, P(O)(OR¹⁰)₂, C1-9 alkyl optionally substituted with one or more OH, OR¹⁰, ═O, NH₂, N₃, halo, CN, NO₂, NR¹⁰R¹¹, CO₂R¹⁰, aryl or carbocyclyl groups.

4. The compound of claim 1 wherein R¹ represents C1-9 alkyl substituted with one or more aryl groups.

5. The compound of claim 4 wherein R¹ represents propyl phenyl or propyl methoxyphenyl.

6. The compound of claim 1 wherein R¹ represents a substituent selected from:
hydrogen
methyl hexyl
4-methyl pentyl
ethyl
ethyl cyclohexyl
propyl
propyl phenyl
propyl methoxyphenyl
propyl chlorphenyl
propyl (bis)phenyl
propanoyl phenyl
propyl (p-ethylsulfamoyl)phenyl
propyl (p-phenylthio)phenyl
butyl
butyl phenyl
hexyl
6-hydroxy hexyl
hexyldansyl
hexyltosylamide
hexyl biphenylsulfonamide
hexyl phenoxyphenyl sulfonamide
N-Boc propylamine
nonyl
hydroxynonyl
methoxyethoxyethyl
ethylphenyl
benzyl
benzyl phenyl
phenoxy ethyl
ethoxy dichlorophenyl.

7. The compound of claim 1 wherein R² represents C(O)NR¹⁰R¹¹; CR¹⁰R¹¹C(O)NR¹⁰R¹¹; NR¹⁰C(O)NR¹⁰R¹¹ or CR¹⁰R¹¹NR¹⁰C(O)NR¹⁰R¹¹.

8. The compound of claim 1 wherein R¹⁰ represents a substituent selected from:
methyl
ethyl
hydroxyethyl
propyl
isopropyl
cyclopropyl
butyl
isobutyl
allyl
methylcyclohexyl
ethyl urea
glycolamide.

9. The compound of claim 1 wherein one from R³, R⁴, R⁵, R⁶, R⁷, R⁸, R⁹ is connected to R² to form an additional ring structure.

10. The compound of claim 1 wherein one from R³, R⁴, R⁵, R⁶, R⁷, R⁸, R⁹ is connected to R¹ to form an additional ring structure.

11. The compound of claim 1 wherein R² is connected to R¹ to form an additional ring structure.

12. The compound of claim 1 wherein one or more endocyclic carbon atom(s) is replaced with a sulphur, oxygen or nitrogen heteroatom.

13. A compound having the formula: 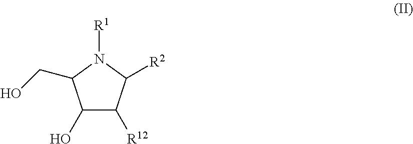
in which
R¹ is H; C1-15 alkyl, C1-15 alkenyl or C1-15 alkynyl, optionally substituted with one or more R³; oxygen or an oxygen containing group such that the compound is an N-oxide; C(O)OR¹⁰; C(O)N R¹⁰R¹¹; SO₂NR¹⁰; SO₂R¹⁰; OH; OR¹⁰; or formyl;
R² is C(O)NR¹⁰R¹¹; CR¹⁰R¹¹C(O)NR¹⁰R¹¹; C(S)NR¹⁰R¹¹; CR¹⁰R¹¹C(S)NR¹⁰R¹¹; CF₂NR¹⁰R¹¹; CR¹⁰R¹¹CF₂NR¹⁰R¹¹; C(R¹⁰)═CR¹⁰R¹¹; CR¹⁰R¹¹C(R¹⁰)═CR¹⁰R¹¹; SO₂NR¹⁰R¹¹; CR¹⁰R¹¹SO₂NR¹⁰R¹¹; NR¹⁰C(O)NR¹⁰R¹¹; CR₁₀R¹¹NR¹⁰C(O)NR¹⁰R¹¹; NR¹⁰C(═NR¹⁰)NR¹⁰R¹¹; CR¹⁰R¹¹NR¹⁰C(═NR¹⁰)NR¹⁰R¹¹; CR¹⁰(CF₃)NR¹⁰R¹¹; NR¹⁰C(O)R¹⁰; NR¹⁰C(S)R¹⁰; CR¹⁰R¹¹NR¹⁰C(O)R¹⁰; CR¹⁰R¹¹NR¹⁰C(S)R¹⁰; NR¹⁰SO₂R¹⁰; CR¹⁰R¹¹NR¹⁰SO₂R¹⁰; NR¹⁰C(CF₃)R¹⁰; CR¹⁰R¹¹NR¹⁰C(CF₃)R¹⁰; NR¹⁰CF₂R¹⁰; CR¹⁰R¹¹NR¹⁰CF₂R¹⁰;
R³ is selected independently from each other from a group consisting of H; OH; OR¹⁰; ═O; NH₂; N₃; SH; SOₓR¹⁰; halo; CN; NO₂; NR¹⁰R¹¹; (NR¹⁰)NR¹⁰R¹¹; NH(NR¹⁰)NR¹⁰R¹¹; CO₂R¹⁰; OC(O)R¹⁰; P(O)(OR¹⁰)₂, C1-15 alkyl or alkenyl optionally substituted with one or more OH, OR¹⁰, ═O, NH₂, N₃, SH, SOₓR¹⁰, halo, CN, NO₂, NR¹⁰R¹¹, (NR¹⁰)NR¹⁰R¹¹, NH(NR¹⁰)NR¹⁰R¹¹, CO₂R¹⁰, OC(O)R¹⁰, P(O)(OR¹⁰)₂, aryl or carbocyclyl groups; carbocyclyl or aryl, either of which is optionally substituted with one or more OH, OR¹⁰, ═O, NH₂, N₃, SH, SOₓR¹⁰, halo, CN, NO₂, NR¹⁰R¹¹, (NR¹⁰)NR¹⁰R¹¹, NH(NR¹⁰)NR¹⁰R¹¹, CO₂R¹⁰, OC(O)R¹⁰, P(O)(OR¹⁰)₂, C1-9 alkyl optionally substituted with one or more OH, OR¹⁰, ═O, NH₂, N₃, halo, CN, NO₂, NR¹⁰R¹¹, CO₂R¹⁰, aryl or carbocyclyl groups; O-glycosyl; C-glycosyl; O-sulfate; O-phosphate or a group which together with the endocyclic carbon forms a spiro ring;
R¹⁰ represents H; C1-6 alkyl, optionally substituted with one or more OH, halo; aryl or C1-4 alkyl optionally substituted with aryl;
R¹¹ represents H; C1-6 alkyl, optionally substituted with one or more OH;
R¹⁰ and R¹¹ may optionally form a 3 to 8 membered ring, containing one or more O, SOₓ or NR¹⁰ groups;
x represents an integer from 0 to 2;
R¹² represents H; halo (e.g. fluoro); or methoxy;
or a pharmaceutically acceptable salt, derivative, solvate, isomer, tautomer, N-oxide, ester, prodrug, isotope or protected form thereof.

14. The compound of claim 13 having the formula: 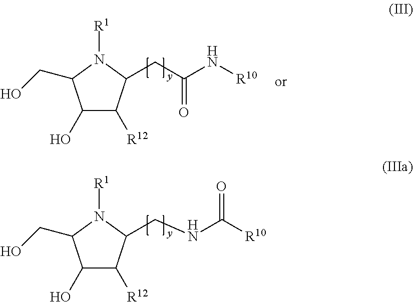
in which
R¹⁰ is H; C1-6 alkyl, optionally substituted with one or more OH, halo; aryl or C1-4 alkyl optionally substituted with aryl;
y represents 0 or 1;
or a pharmaceutically acceptable salt, derivative, solvate, isomer, tautomer, N-oxide, ester, prodrug, isotope or protected form thereof.

15. The compound of claim 14 having the formula: 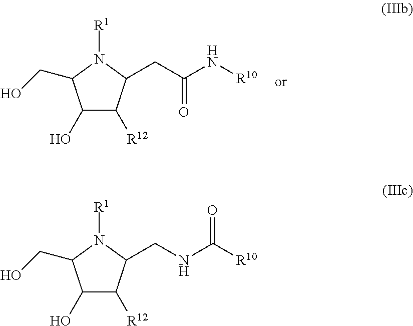
or a pharmaceutically acceptable salt, derivative, solvate, isomer, tautomer, N-oxide, ester, prodrug, isotope or protected form thereof.

16. The compound of claim 1 having the formula: 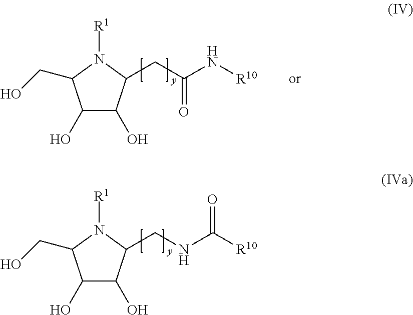
in which
y represents 0 or 1;
or a pharmaceutically acceptable salt, derivative, solvate, isomer, tautomer, N-oxide, ester, prodrug, isotope or protected form thereof.

17. The compound of claim 16 having the formula: 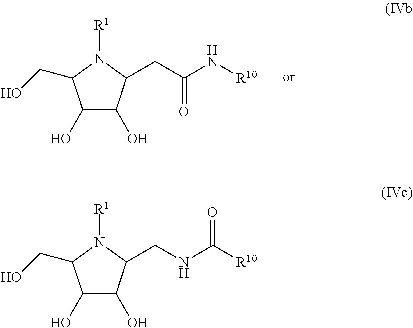
or a pharmaceutically acceptable salt, derivative, solvate, isomer, tautomer, N-oxide, ester, prodrug, isotope or protected form thereof.

18. The compound of claim 13 having the formula: 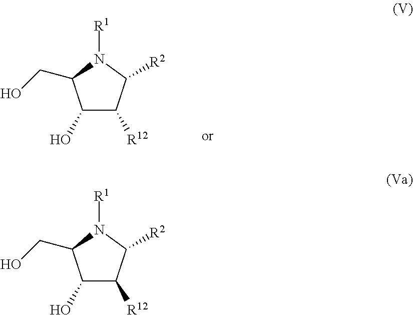
or a pharmaceutically acceptable salt, derivative, solvate, isomer, tautomer, N-oxide, ester, prodrug, isotope or protected form thereof.

19. The compound of claim 14 having the formula: 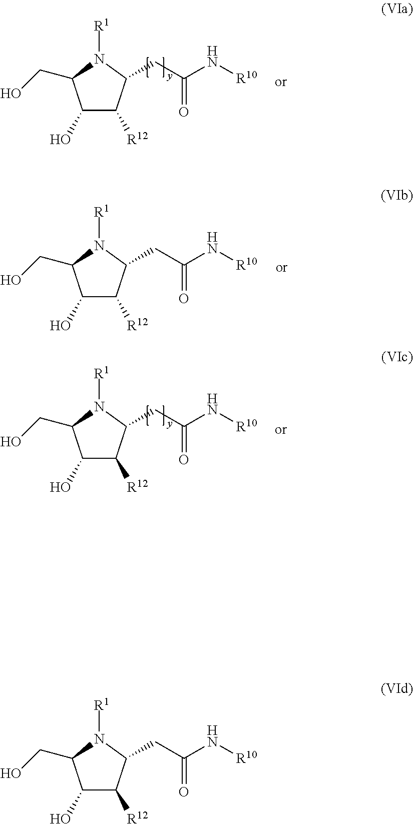
or the reverse amide thereof in which the moiety: 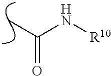
is replaced with: 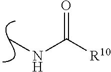
or a pharmaceutically acceptable salt, derivative, solvate, isomer, tautomer, N-oxide, ester, prodrug, isotope or protected form thereof.

20. The compound of claim 16 having the formula: 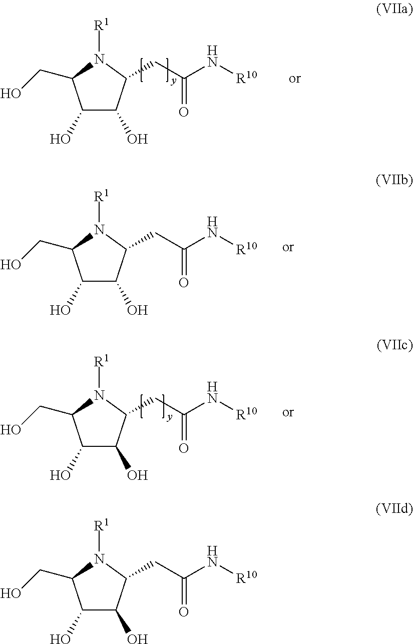
or the reverse amide thereof in which the moiety: 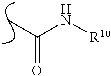
is replaced with: 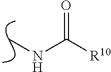
or a pharmaceutically acceptable salt, derivative, solvate, isomer, tautomer, N-oxide, ester, prodrug, isotope or protected form thereof.

21. The compound of claim 1 which is selected from compounds listed in Table 1, or a pharmaceutically acceptable salt, derivative, solvate, isomer, tautomer, N-oxide, ester, prodrug, isotope or protected form thereof.

22. The compound of claim 1 which is an OGA inhibitor.

23. The compound of claim 22 which is a selective OGA inhibitor.

24. A pharmaceutical composition, pharmaceutical kit or patient pack comprising the compound as defined in claim 1.

25-36. (canceled)
